SEQUENCE LISTING (58 sequences)

SEQ ID NO: 1 - protein, 352 aa, Artificial sequence
  Synthetic
NTKYNEEFLLYLAGFVDGDGSIIAQIKPNQSYKFKHQLSLTFMVTQKTQRRWFLDKLVDEIGVGYVRDAGSVSNYILSKIKPLHNFLTQLQPFLELKQKQANLALKIIEQLPSAKESPDKFLEVCTWVDQVAALNDSKTRKTTSKTVRAVLDSLGEKKKSSPAAGGSDKYNQALSKYNQALSKYNQALSGGGGSNKKFLLYLAGFVDSDGSIIAQIKPNQEYKFKHYLSLTFAVTQKTQRRWFLDKLVDRIGVGYVYDSGSVSYYKLSKIKPLHNFLTQLQPFLKLKQKQANLALKIIEQLPSAKESPDKFLEVCTWVDQVAALNDSKTRKTTSETVRAVLDSLSEKKKSSP

SEQ ID NO: 2 - protein, 348 aa, Artificial sequence
  Synthetic
ANTKYNKEFLLYLAGFVDGDGSIIAQIKPNQSYKFKHQLSLTFDVTQKTQRRWFLDKLVDEIGVGYVTDSGSVSRYRLSEIKPLHNFLTQLQPFLKLKQKQANLVLKIIEQLPSAKESPDKFLEVCTWVDQIAALNDSKTRKTTSETVRAVLDSLSEKKKSSPAAGDSSVSNSEHIAPLSLPSSPPSVGSNKEFLLYLAGFVDSDGSIIAQIEPNQSYKFKHRLKLTFKVTQKTQRRWILDKLVDEIGVGYVRDSGSVSNYILSEIKPLHNFLTQLQPFLKLKQKQANLALKIIEQLPSAKESPDKFLEVCTWVDQVAALNDSKTRKTTSETVRAVLDSLSEKKKSSP

SEQ ID NO: 3 - protein, 353 aa, Artificial sequence
  Synthetic
ANTKYNEEFLLYLAGFVDGDGSIIAQIKPKQSAKFKHQLSLTFQVTQKTQRRWFLDKLVDEIGVGYVRDSGSVSHYYLSEIKPLHNFLTQLQPFLELKQKQANLVLKIIEQLPSAKESPDKFLEVCTWVDQVAALNDSKTRKTTSETVRAVLDSLSEKKKSSPAAGGSDKYNQALSKYNQALSKYNQALSGGGGSNKKFLLYLAGFVDSDGSIIAQIKPNQHCKFKHQLALTFQVTQKTQRRWFLDKLVDRIGVGYVRDSGSVSNYKLSEIKPLHNFLTQLQPFLKLKQKQANLVLKIIEQLPSAKESPDKFLEVCTWVDQVAALNDSKTRKTTSETVRAVLDSLSEKKKSSP

SEQ ID NO: 4 - DNA, 24 bp, Artificial sequence
  Synthetic
ataaactcacacacggcgtcctgg

SEQ ID NO: 5 - DNA, 24 bp, Artificial sequence
  Synthetic
ccaagctggtgtacctgatagtgg

SEQ ID NO: 6 - DNA, 24 bp, Artificial sequence
  Synthetic
ccgctctgttttaccgcgtctacg

SEQ ID NO: 7 - DNA, 39 bp, Artificial sequence
  Synthetic
catggccttggcggtcgatgcggcccgaggattgccggc

SEQ ID NO: 8 - DNA, 37 bp, Artificial sequence
  Synthetic
catggccttggcggtcgatgcggcgaggattgccggc

SEQ ID NO: 9 - DNA, 33 bp, Artificial sequence
  Synthetic
catggccttggcggtcgcccgaggattgccggc

SEQ ID NO: 10 - DNA, 35 bp, Artificial sequence
  Synthetic
catggccttggcggtcgatgcggaggattgccggc

SEQ ID NO: 11 - DNA, 35 bp, Artificial sequence
  Synthetic
catggccttggcggtcgatcccgaggattgccggc

SEQ ID NO: 12 - DNA, 29 bp, Artificial sequence
  Synthetic
catggccttggcggtcgaggattgccggc

SEQ ID NO: 13 - DNA, 36 bp, Artificial sequence
  Synthetic
catggccttggcggtcgatgcccgaggattgccggc

SEQ ID NO: 14 - DNA, 38 bp, Artificial sequence
  Synthetic
catggccttggcggtcgatgcggccgaggattgccggc

SEQ ID NO: 15 - DNA, 37 bp, Artificial sequence
  Synthetic
catggccttggcggtcgatgccccgaggattgccggc

SEQ ID NO: 16 - DNA, 31 bp, Artificial sequence
  Synthetic
catggccttggcggtcgatgcggttgccggc

SEQ ID NO: 17 - DNA, 34 bp, Artificial sequence
  Synthetic
catggccttggcggtcgatgcgaggattgccggc

SEQ ID NO: 18 - DNA, 38 bp, Artificial sequence
  Synthetic
catggccttggcggtcgatgcgcccgaggattgccggc

SEQ ID NO: 19 - DNA, 36 bp, Artificial sequence
  Synthetic
catggccttggcggtcgatgcgcgaggattgccggc

SEQ ID NO: 20 - DNA, 30 bp, Artificial sequence
  Synthetic
catggccttggcggtcgatggattgccggc

SEQ ID NO: 21 - DNA, 32 bp, Artificial sequence
  Synthetic
catggccttggcggtcgatgcggattgccggc

SEQ ID NO: 22 - DNA, 34 bp, Artificial sequence
  Synthetic
catggccttggcggtcgatgcggggattgccggc

SEQ ID NO: 23 - DNA, 26 bp, Artificial sequence
  Synthetic
catggccttggcggaggattgccggc

SEQ ID NO: 24 - DNA, 34 bp, Artificial sequence
  Synthetic
catggccttggcggtcgacccgaggattgccggc

SEQ ID NO: 25 - DNA, 33 bp, Artificial sequence
  Synthetic
catggccttggcggtcgatgcgggattgccggc

SEQ ID NO: 26 - DNA, 32 bp, Artificial sequence
  Synthetic
catggccttggcggtcgacgaggattgccggc

SEQ ID NO: 27 - DNA, 36 bp, Artificial sequence
  Synthetic
catggccttggcggtcgatgcgggaggattgccggc

SEQ ID NO: 28 - DNA, 30 bp, Artificial sequence
  Synthetic
catggccttggcggtcgatgcggtgccggc

SEQ ID NO: 29 - DNA, 35 bp, Artificial sequence
  Synthetic
catggccttggcggtcgatgccgaggattgccggc

SEQ ID NO: 30 - DNA, 34 bp, Artificial sequence
  Synthetic
catggccttggcggtcgatgcggcgattgccggc

SEQ ID NO: 31 - DNA, 31 bp, Artificial sequence
  Synthetic
catggccttggcggtcgataggattgccggc

SEQ ID NO: 32 - DNA, 29 bp, Artificial sequence
  Synthetic
catggccttggcggtcgatgcttgccggc

SEQ ID NO: 33 - DNA, 28 bp, Artificial sequence
  Synthetic
catggccttggcggtcgatattgccggc

SEQ ID NO: 34 - DNA, 28 bp, Artificial sequence
  Synthetic
catggccttggcggtcgagattgccggc

SEQ ID NO: 35 - DNA, 24 bp, Artificial sequence
  Synthetic
catggccttggcggtcttgccggc

SEQ ID NO: 36 - DNA, 25 bp, Artificial sequence
  Synthetic
catggccttggcggtcattgccggc

SEQ ID NO: 37 - DNA, 20 bp, Artificial sequence
  Synthetic
ctggccgtggtcgtacatga

SEQ ID NO: 38 - DNA, 20 bp, Artificial sequence
  Synthetic
tcaccgacatgggcaacctt

SEQ ID NO: 39 - DNA, 24 bp, Artificial sequence
  Synthetic
gagaacgtggagcacgcgtacggc

SEQ ID NO: 40 - DNA, 24 bp, Artificial sequence
  Synthetic
ggcccggtttgagacggtaccagc

SEQ ID NO: 41 - DNA, 20 bp, Artificial sequence
  Synthetic
gacagcacggacacggaact

SEQ ID NO: 42 - DNA, 20 bp, Artificial sequence
  Synthetic
tcgtccaggtcgtcgtcatc

SEQ ID NO: 43 - DNA, 20 bp, Artificial sequence
  Synthetic
gaccgcatcagcgagagctt

SEQ ID NO: 44 - DNA, 20 bp, Artificial sequence
  Synthetic
ctcgcagacacgactcgaac

SEQ ID NO: 45 - DNA, 20 bp, Artificial sequence
  Synthetic
cggccatcaagaagtacgag

SEQ ID NO: 46 - DNA, 20 bp, Artificial sequence
  Synthetic
aagtggctctggcctatgtc

SEQ ID NO: 47 - DNA, 18 bp, Artificial sequence
  Synthetic
ggcgttcgcagatttgga

SEQ ID NO: 48 - DNA, 20 bp, Artificial sequence
  Synthetic
tcccaggtgagcagcagtct

SEQ ID NO: 49 - DNA, 23 bp, Artificial sequence
  Synthetic
acctgaaggctctgcgcggactc

SEQ ID NO: 50 - DNA, 20 bp, Artificial sequence
  Synthetic
ccgcccggaaagagattatt

SEQ ID NO: 51 - DNA, 21 bp, Artificial sequence
  Synthetic
cggagttcagattggtcagtt

SEQ ID NO: 52 - DNA, 24 bp, Artificial sequence
  Synthetic
agctgctggatcagattgccaaga

SEQ ID NO: 53 - DNA, 22 bp, Artificial sequence
  Synthetic
tgccgtgtttatctcgtcaact

SEQ ID NO: 54 - DNA, 25 bp, Artificial sequence
  Synthetic
cccgccatgctacttatctacttaa

SEQ ID NO: 55 - DNA, 14 bp, Artificial sequence
  Synthetic
ttggcgagattttt

SEQ ID NO: 56 - DNA, 19 bp, Artificial sequence
  Synthetic
tggacagcctgagcgagaa

SEQ ID NO: 57 - DNA, 22 bp, Artificial sequence
  Synthetic
gcagagacagaggagcaatgtg

SEQ ID NO: 58 - DNA, 28 bp, Artificial sequence
  Synthetic
cggccggtgattcctctgtttctaattc

(19) United States
(12) Patent Application Publication
(10) Pub. No.: US 2024/0301372 A1
(43) Pub. Date: Sep. 12, 2024

(54) TREATMENT FOR HSV-1 USING A MEGANUCLEASE

(71) Applicant: FRED HUTCHINSON CANCER CENTER, Seattle, WA (US)

(72) Inventors: Keith R. Jerome, Seattle, WA (US); Barry L. Stoddard, Seattle, WA (US)

(73) Assignee: FRED HUTCHINSON CANCER CENTER, Seattle, WA (US)

(21) Appl. No.: 18/262,674

(22) PCT Filed: Jan. 25, 2022
(86) PCT No.: PCT/US2022/013757

§ 371 Date: Jul. 24, 2023

Related U.S. Application Data
(60) Provisional application No. 63/141,344, filed on Jan. 25, 2021.
(60) Provisional application No. 63/176,813, filed on Apr. 19, 2021.

(51) Int. Cl. C12N 9/22; A61K 9/00; A61K 31/437; A61K 31/517; A61K 31/5383; A61K 31/5517; A61K 38/00; A61P 31/22; C12N 15/86

(52) U.S. Cl. CPC C12N 9/22; A61K 9/0019; A61K 31/437; A61K 31/517; A61K 31/5383; A61K 31/5517; A61P 31/22; C12N 15/86; A61K 38/00; C12N 2750/14143

(57) ABSTRACT

Embodiments of the present disclosure are directed to methods and compositions for reducing or eliminating latent HSV-1 reactivation in a cell. In some embodiments, the method comprises delivering to an HSV-1-infected cell one or more self-complementary adeno-associated viruses (scAAV) comprising one or more sequences encoding one or more HSV-1-specific meganucleases. In some embodiments, the composition comprises one or more self-complementary adeno-associated viruses (scAAV) comprising one or more sequences encoding one or more HSV-1-specific meganucleases.

### CROSS-REFERENCE(S) TO RELATED APPLICATION(S)

This application claims the benefit of U.S. Provisional Application No. 63/141,344, filed Jan. 25, 2021 and U.S. Provisional Application No. 63/176,813, filed Apr. 19, 2021, the disclosures of which are incorporated herein in its entirety.

### STATEMENT OF GOVERNMENT LICENSE RIGHTS

This invention was made with Government support under AI132599 AND GM105691 awarded by the National Institutes of Health. The Government has certain rights in the invention.

### STATEMENT REGARDING SEQUENCE LISTING

The sequence listing associated with this application is provided in text format in lieu of a paper copy and is hereby incorporated by reference into the specification. The name of the text file containing the sequence listing is 1896-P58WO_Seq_List_FINAL_20220119_ST25.txt. The text file is 20 KB; was created on Jan. 19, 2022; and is being submitted via EFS-Web with the filing of the specification.

### BACKGROUND

Herpes simplex virus type 1 (HSV-1) is widespread and important human pathogens, causing oral and genital ulcers, neonatal herpes, and increasing the risk of acquiring HIV. After primary infection at the skin or mucosa, HSV-1 establishes lifelong latency in both sensory (e.g., trigeminal and dorsal root ganglia) and autonomic (e.g., superior cervical and major pelvic ganglia) neurons of the peripheral nervous system. HSV-1 can subsequently reactivate from the latent state, causing lesions and/or virus shedding at mucosal surfaces. While current antiviral therapies reduce the severity of acute infections and diminish viral reactivation frequency, they do not reduce or eliminate the latent virus that drives recurrent disease. Gene editing using CRISPR/Cas9, meganucleases, or similar enzymes offers the possibility of directly targeting latent genomes for disruption or elimination while preserving neurons, thus eliminating the possibility of viral reactivation and pathogenesis.

In vivo gene editing of latent HSV genomes within TG sensory neurons of mice has been previously demonstrated using HSV-specific meganucleases delivered via adeno-associated virus (AAV) vectors, but the levels of gene editing were modest (<4%). In view of the limitations of the present art, a need remains for antiviral therapies that reduce or eliminate the latent virus that causes recurrent disease. The present disclosure addresses these and related needs.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

Disclosed herein are embodiments of methods and compositions for reducing or eliminating latent herpes simplex virus type 1 (HSV-1) reactivation in a cell.

In one aspect, the method for reducing or eliminating latent HSV-1 reactivation in a cell can comprise delivering to an HSV-1-infected cell one or more viral vectors comprising one or more sequences encoding one or more HSV-1-specific meganucleases.

In another aspect, the composition for reducing or eliminating latent HSV-1 reactivation in a cell can comprise one or more viral vectors comprising one or more sequences encoding one or more HSV-1-specific meganucleases.

In some embodiments, the one or more viral vectors is a self-complementary adeno-associated virus (scAAV) and/or a single-stranded adeno-associated virus (ssAAV). In still other embodiments, the one or more viral vectors is a scAAV. In some embodiments, the one or more scAAVs can comprise AAV-Rh10, AAV8, AAV1 serotype adeno-associated virus, or a combination thereof. In still other embodiments, the one or more scAAVs is AAV-Rh10 or AAV8 serotype adeno-associated virus.

In some embodiments, the one or more HSV-1-specific meganucleases can be configured to induce one or more DNA double strand breaks (DSB). In some embodiments, the one or more HSV-1-specific meganucleases is a meganuclease that can be configured to target UL19 encoding major capsid protein VP5. In some embodiments, the one or more HSV-1-specific meganucleases is a meganuclease that can be configured to target UL30 encoding the catalytic subunit of an HSV-1 DNA polymerase. In some embodiments, the one or more HSV-1-specific meganucleases is a meganuclease that can be configured to target the duplicated gene ICP0. In some embodiments, the one or more HSV-1-specific meganucleases is a meganuclease that can be configured to target UL54 encoding immediate early regulatory protein ICP27. In some embodiments, the one or more HSV-1-specific meganucleases is a meganuclease that can be configured to target any combination of UL19, UL30, ICP0, and/or UL54. In some embodiments, the one or more meganucleases can comprise a sequence as set forth in SEQ ID NOs: 1-3. In some embodiments, the one or more meganuclease is a meganuclease that can be configured to target one or more sequences as set forth in SEQ ID NOs: 4-6.

In some embodiments, the method can comprise delivering one scAAV comprising two or more sequences encoding one or more HSV-1-specific meganucleases. In some embodiments, the method can comprise delivering two scAAVs each comprising one or more sequences encoding one or more HSV-1-specific meganucleases. In still other embodiments, the method can comprise delivering two different scAAVs each comprising a sequence encoding an HSV-1-specific meganuclease, wherein the sequences are the same or different. In still other embodiments, the one or more scAAVs can be delivered to the subject by a subcutaneous injection.

In some embodiments, the cell can be in a mammalian subject. In some embodiments, the mammalian subject can be human. In still other embodiments, the cell can be a superior cervical ganglia (SCG) cell. In still other embodiments, the cell can be a trigeminal ganglia (TG) cell. In still other embodiments, the cell can be a combination of a SCG cell and/or a TG cell.

In some embodiments, the method further comprises administering to the HSV-1-infected cell a bromodomain and extra-terminal (BET) protein inhibitor. In some embodiments, the method can comprise administering the BET protein inhibitor to the HSV-1-infected cell before delivering to the HSV-1-infected cell one or more scAAVs comprising one or more sequences encoding one or more HSV-1-specific meganucleases. In some embodiments, the method can comprise administering the BET protein inhibitor to the HSV-1-infected cell after delivering to the HSV-1-infected cell one or more scAAVs comprising one or more sequences encoding one or more HSV-1-specific meganucleases. In still other embodiments, the method can comprise administering the BET protein inhibitor to the HSV-1-infected cell concomitant with delivering to the HSV-1-infected cell one or more scAAVs comprising one or more sequences encoding one or more HSV-1-specific meganucleases.

In some embodiments, the BET protein inhibitor can be administered at a dose sufficient to provide a concentration of 3 μM or less in the HSV-1-infected cell. In some embodiments, the BET protein inhibitor can be selected from the group of JQ1, birabresib, molibresib, apabetalone, ZEN-3694, BMS-986158, and INC-B057643.

In some embodiments, the method can reduce HSV-1 load at least 97% in SCG cells at least 96 hours following administration of the BET protein inhibitor. In some embodiments, the method can reduce HSV-1 load at least 83% in SCG cells at least 48 hours following administration of the BET protein inhibitor. In still other embodiments, the method can reduce HSV-1 load at least 97% in TG cells 96 hours following administration of the BET protein inhibitor.

In some embodiments, the composition can comprise one or more pharmaceutically acceptable carriers configured for subcutaneous injection. In some embodiments, the composition can further comprise a bromodomain and extra-terminal (BET) protein inhibitor. In some embodiments, the composition can reduce HSV-1 load at least 97% in SCG cells 96 hours following administration of the BET protein inhibitor. In some embodiment, the composition can reduce HSV-1 load at least 83% in SCG cells 48 hours following administration of the BET protein inhibitor. In still other embodiments, the composition can reduce HSV-1 load at least 97% in TG cells 96 hours following administration of the BET protein inhibitor.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIGS. 1A through 1D. HSV load reduction in dual-meganuclease treated mice. FIG. 1A. Mice latently infected for 44 days with 10⁵ PFU HSV 17+ were left untreated (Controls, circles n=8) or administered by whisker pad injection either 5×10¹¹ vector genomes (vgs) scAAV8-CBh-m5 (squares, n=9), 5×10¹¹ vgs scAAV8-CBh-m8 (triangles, n=8) or 5×10¹¹ vgs of each scAAV8-CBh-m5 and scAAV8-CBh-m8 (diamonds, n=9). Analysis was performed 31 days later. FIG. 1B. ddPCR quantification of HSV genomes in superior cervical ganglia (SCG) and right (ipsilateral) trigeminal ganglia (TGs) from infected mice (p=0.018 in SCG of controls vs m5+m8; p=0.001 in TG of controls vs m5+m8). FIGS. 1C-1D. NGS analysis of m5 and m8 sites in HSV genomes from SCGs and right (ipsilateral) TGs from infected mice treated with either m5 (squares, n=9), m8 (triangles, n=8) or m5+m8 (diamonds, n=9). ns: not significantly different from controls, *p<0.05, **p<0.01, ***p<0.001, significantly different from controls. All data are presented as mean values+/−SD. Statistical analysis was conducted using unpaired multiple t-Test without correction for multiple comparison.

FIGS. 2A through 2F. Efficient gene editing after optimized delivery of dual-meganuclease therapy. FIG. 2A. Mice latently infected with 10⁵ PFU HSV 17+ for 30 days, were administered 0.5-1×10¹² scAAV-Rh10-CBh-m5 by retroorbital (RO) injection. Analysis was performed 41 days later. FIG. 2B. ddPCR quantification of HSV genomes in superior cervical ganglia (SCG) and right (ipsilateral) trigeminal ganglia (TG) of control (CTRL, circles n=3) or m5 (squares n=6) treated mice. p=0.017 for SCG. FIG. 2C. Next generation sequencing (NGS) analysis in SCG and TG from m5-treated mice (n=6) to detect HSV gene editing at the m5 target site in SCG (circles) or TG (squares). FIG. 2D. Mice latently infected with 10⁵ PFU HSV 17+ in the right eye for 28 days were administered 5×10¹¹ vector genomes (vgs) scAAV-Rh10-CBh-m5+5×10¹¹ vgs scAAV-Rh10-CBh-m8 by RO injection. SCGs and ipsilateral TG were collected 33-35 days later. FIG. 2E. ddPCR quantification of HSV genomes in TG and SCG from latently infected mice either left untreated (Controls, CTRL circles, n=12) or administered m5+m8 (squares, n=12). p=0.006 and p=0.01 for SCG and TG, respectively. FIG. 2F. NGS analysis of SCG and TG from treated mice to detect mutations at either the m5 (closed circles) or m8 (open circles) target sites. ns: not significantly different from controls, *p<0.05, **p<0.01, ***p<0.001, significantly different from controls. All data are presented as mean values+/−SD. Statistical analysis was conducted using unpaired multiple t-Test without correction for multiple comparison. Source data are provided as a Source Data file.

FIGS. 3A through 3F. Reduction of ganglionic HSV genomes after dual-meganuclease therapy. FIG. 3A. Mice latently infected with 10⁵ PFU HSV 17+ for 28 days, were either left untreated (Controls, CTRL, n=12) or administered 1×10¹² vector genomes (vgs) scAAV-Rh10-CBh-m4 (m4, n=12) by retroorbital (RO) injection. These mice were infected and treated at the same time as the mice described in FIGS. 2D-F (control mice are the same for these 2 data sets). At 33-35 days post meganuclease-therapy, superior cervical ganglia (SCGs) and right (ipsilateral) trigeminal ganglia (TG) were harvested and FIG. 3B, HSV genomes quantified by ddPCR. p=0.03 and p=0.02 for SCG and TG, respectively. FIG. 2C. Next generation sequencing (NGS) analysis of SCG and TG from dual meganuclease-treated mice to detect HSV gene editing at the m4 target site in SCG or TG. FIG. 3D. Mice latently infected with 10⁵ PFU HSV 17+ for 28 days were either left untreated (Controls, CTRL, n=10) or administered 5×10¹¹ vgs scAAV-Rh10-CBh-m5+5×10¹¹ vgs scAAV-Rh10-CBh-m4 (m5+m4, n=10) by RO injection. At 40-41 days post meganuclease-therapy, SCGs and right (ipsilateral) TG were harvested from infected mice either untreated (closed circles) or treated with m5+m4 (closed squares) and FIG. 3E, HSV genomes quantified by ddPCR, p=0.00001 for SCG. FIG. 3F. NGS analysis of SCG and TG from dual meganuclease-treated mice to detect HSV gene editing at m5 (open circles) and m4 (closed circles) target sites in SCG or TG. ns: not significantly different from controls, *p<0.05, **p<0.01, ***p<0.001, significantly different from controls. All data are presented as mean values+/−SD. Statistical analysis was conducted using unpaired multiple t-tests without correction for multiple comparison. Source data are provided as a Source Data file.

FIGS. 4A through 4E. Reactivation after dual-meganuclease therapy. FIG. 1A. Ganglia from a second set of mice latently infected and treated with dual-meganuclease therapy at the same time as those described in FIGS. 2D-F, were subjected to ganglia (SCG/TG) explant reactivation (see Methods) prior to DNA extraction. FIG. 4B. ddPCR quantification of HSV genomes in reactivated superior cervical ganglia (SCG) and trigeminal ganglia (TG) from latently infected untreated control mice (CTRL, open circles, n=12) or dual-meganuclease treated mice (open squares, n=12). p=0.002 and p=0.01 for SCG and TG, respectively. FIG. 4C. Next generation sequencing (NGS) analysis in reactivated SCG and TG from dual-meganuclease treated mice to detect HSV gene editing at either the m5 (m5, closed circles) or m8 (m8, open circles) target sites. FIGS. 4D-4E. Comparison of HSV loads in SCG and TG from latently infected (closed circles) and reactivated (open circles) control mice (CTRL), p=0.02 for SCG from latently infected (closed squares) or reactivated (open squares) dual-meganuclease treated mice (m5+m8), p=0.0012 and p=0.012 for FIG. 4D SCG and FIG. 4E TG, respectively. *p<0.05; **p<0.01; ns: not significantly different. All data are presented as mean values+/−SD. Statistical analysis was conducted using unpaired multiple t-tests without correction for multiple comparison (4C) and one-tail, unpaired t-test (4D, 4E). Source data are provided as a Source Data file.

FIGS. 5A through 5F. SaCas9 gene editing of HSV in infected neuronal cultures. FIG. 5A. Schematic of neuronal culture generation and exposure to AAV/CRISPR-Cas9 treatment. Mice were infected with 2×10⁵ PFU of HSV-1(F); right trigeminal ganglia (TGs) were collected 7 days later. Neuronal cultures were established, and cells were cultured for 5 days in medium supplemented with 100 μM ACV as previously described (Aubert, M. et al. In vivo disruption of latent HSV by designer endonuclease therapy. JCI Insight 1, doi:10.1172/jci.insight.88468 (2016)). Cells were then transduced at the indicated time at a MOI of 10⁶ AAV vector genomes (vgs) per neuron, with either ssAAV1-sCMV-SaCas9-sgRNA_{UL54} or SSAAV1-sCMV-SaCas9-sgRNA_{UL30}. Analysis was performed at 10 days after AAV exposure. Mutagenic event detection by T7E1 assay in DNA from cultured TG neurons treated with either FIG. 5B, 10⁶ vgs ssAAV1-sCMV-SaCas9-sgRNA_{UL54} or FIG. 5C, SSAAV1-sCMV-SaCas9-sgRNA_{UL30}. The HSV regions containing the target site for each sgRNA were PCR amplified from total genomic DNA obtained from the right ipsilateral TG. Products were subjected to T7E1 digestion and separated on a 3% agarose gel. Gel legend: mw: molecular weight size ladder, V: no sgRNA, 13: sgRNA_{UL54-13}, 17: sgRNA_{UL54-17}, 26: sgRNA_{UL54-26}, 1: sgRNA_{UL30-1}, 2: sgRNA_{UL30-2}, 3: sgRNA_{UL30-3}, 6: sgRNA_{UL30-6}, 9: sgRNA_{UL30-9}, 10: sgRNA_{UL30-10}. Schematic representation of PCR amplicon with full-size and T7E1 cleavage product sizes indicative of HSV-specific Cas9 cleavage and mutagenesis is provided below each gel. The location of the sgRNA site in the PCR product is indicated by a black (efficient) or grey (inefficient) arrowhead, and resulting T7 digest products are indicated for efficient sgRNA. FIG. 5D. Mice were latently infected with 2×10⁵ PFU HSV-1(F), and 28 days later were injected in the right whisker pad with 10¹² vgs ssAAV1-sCMV-SaCas9-sgRNA_{UL54} (n=5 per sgRNA_{UL54}). Analysis was performed at either 28 (n=3 mice per sgRNA_{UL54}) or 56 (n=2 mice per sgRNA_{UL54}) days post AAV exposure. FIG. 5E. Levels of genomes were quantified by ddPCR in right (ipsilateral) TGs from infected mice. FIG. 5F. Mutagenic event detection by NGS analysis of the PCR products used in the T7E1 analysis (See FIG. 11A through 11C). sgRNA_{UL54-13} (circles), sgRNA_{UL54-17} (squares), and sgRNA_{UL54-26} (triangles). The gel images were cropped. All data are presented as mean values+/−SD. bp: base pairs. Source data are provided as a Source Data file.

FIGS. 6A through 6K. Dual sgRNA therapy did not increase SaCas9 gene editing efficiency of latent HSV. FIG. 6A. Mice were latently infected with 10⁵ PFU HSV-1(F), and 28 days later were left untreated (CTRL, n=10 circles) or administered by retroorbital (RO) injection either dual sgRNA therapy consisting of 10¹² vector genomes (vgs) ssAAVRh10-sCMV-SaCas9-sgRNA_{UL54-26} and 10¹² vgs ssAAVRh10-sCMV-SaCas9-sgRNA_{UL30-10} (Cas9, n=10 squares) or meganuclease therapy of 10¹² vgs ssAAVRh10-smCBA-m5-Trex2-mCherry (m5+Trex2, n=10 triangles). At 29 days post AAV exposure FIGS. 6B-6C, levels of HSV genomes were quantified by ddPCR in superior cervical ganglia (SCGs) and right (ipsilateral) trigeminal ganglia (TGs) from infected mice. FIGS. 6D-6E. NGS analysis was performed to detect mutation at the site targeted by either sgRNA_{UL54-26}, sgRNA_{UL30-10} or m5 in latent HSV from SCG and TG of treated mice. FIGS. 6F-6G. Detection of Cas9 mRNA by RT-qPCR in SCG (6J) and TG (6K) of infected mice. FIGS. 6H-6I. Detection of sgRNA by RT-ddPCR in SCG (6H) and TG (6I) of infected mice. FIGS. 6J-6K. Detection of m5 mRNA by RT-qPCR in SCG (6J) and TG (6K) of infected mice. All data are presented as mean values+/−SD. Source data are provided as a Source Data file.

FIGS. 7A through 7D. Single cell RNA-seq analysis of purified neurons. FIG. 7A. tSNE plot of neurons dividing them into transcriptionally defined clusters. Clusters of the neurons purified from the superior cervical ganglia (SCG) (n=2,041 with 94,797 mean reads and 5,635 median genes per cell) illustrated with grey/dark grey dots in top grouping (SCG-1-5), and neurons purified from the trigeminal ganglia (TG) (n=2,319 with 99,817 mean reads and 5,908 median genes per cell) are illustrated with grey/dark grey dots in bottom grouping (TG-1-10). Lymphocytes are illustrated with grey dots at the bottom of the figure. FIGS. 7B-7C. Fractional distribution of each neuronal cluster within all neurons, or neurons expressing HSV RNA or the transgenes carried by the indicated AAV serotype, across the SCG and TG. FIG. 7D. Calculated percentage of HSV+ cells in the SCG and TG that also express a given transgene mScarlet (AAV1+); mEGFP (AAV8+); DsRed.Express2 (PHP.S+); TagBFP2 (Rh10+).

FIGS. 8A through 8I. AAV serotype combination for the delivery of dual-meganuclease therapy. FIG. 8A. Mice latently infected with 10⁵ PFU HSV 17+ for 57 days, were either left untreated (Controls, CTRL, n=10) or administered 2×10¹² vector genomes (vgs) total of scAAV-CBH-meganuclease combination by retroorbital (RO) injection to deliver meganuclease dual therapy (m5+m8, n=10 per AAV combination). Mice received either single (Rh10, 1, or 8), dual (1-8, 1-Rh10 or 8-Rh10) or triple (1-8-Rh10) AAV serotype combination (Table 6). At 33-36 days post meganuclease-therapy, superior cervical ganglia (SCGs) and right (ipsilateral) trigeminal ganglia (TG) were harvested, and FIGS. 8B-8C, AAV genomes and FIGS. 8D-8E HSV genomes from SCG (B-D) and TG (C-E) from infected mice were quantified by ddPCR. Percentages of HSV genomes decrease in ganglia from dual-meganuclease treated mice compare with control untreated (CTRL) mice are indicated for each AAV combination (D-E). n/a: not applicable. FIG. 8F-8I. Gene editing at the m5 target (F-G) site and m8 target site (H-I) were quantified by T7E1 assay in SCG (F-H) or TG (G-I). ns: not significantly different from controls, *p<0.05, **p<0.01, ***p<0.001, ****p<0.0001 significantly different from controls. All data are presented as mean values+/−SD. Statistical analysis was conducted using one-tail, unpaired t-test. Exact p values are indicated in Table 6. Source data are provided as a Source Data file.

FIGS. 9A through 9H. Screening of AAV serotypes and route of administration for nuclease delivery to ganglionic neurons. FIG. 9A. Mice were infected with 1-2×10⁵ PFU HSV 17+ in the right eye following corneal scarification, and 30-46 days later after HSV latency establishment, were administered 5×10¹¹ vector genomes (vgs) scAAVCBh-HSV1m5 packaged with AAV serotype 8, PHP.S or Rh10 by either tail vein (TV, circles, n=3), retroorbital (RO, squares, n=3), or whisker pad (WP, triangles, n=3) injection. Analysis was performed 38-41 days later. FIG. 9B. AAV and FIG. 9C HSV genomes from right ipsilateral superior cervical ganglia (SCG) (top panels) or trigeminal ganglia (TG) (bottom panels) were quantified by ddPCR. FIG. 9D. Mutagenic events at the HSV1m5 target site in HSV genomes in SCG (top panel) or TG (bottom panel) were quantified by NGS analysis. FIG. 9E. Mice infected with 10⁵ PFU HSV 17+ in the right eye following corneal scarification for 30 days, were administered 5×10¹¹ vgs scAAV1-CBh-HSV1m5 by either TV (circles, n=3), RO (squares, n=3) or WP (triangles, n=3) injection. At 41 days post AAV administration, ipsilateral SCG and TG were collected for analysis. FIG. 9F AAV and FIG. 9G HSV genomes from SCG and TG were quantified by ddPCR. FIG. 9H. Mutagenic events at the HSV1m5 target site in HSV genomes in SCG and TG were quantified by NGS analysis. All data are presented as mean values+/−SD. Source data are provided as a Source Data file.

FIGS. 10A through 10B. HSV loads after tissue explant reactivation. FIG. 10A. Mice were infected with 2×10⁵ PFU HSV 17+ in the right eye following corneal scarification, and 36 days later right ipsilateral trigeminal ganglia (TG) was collected and total DNA extracted either immediately or after being subjected to TG explant reactivation by placing them into culture media for 22 h. FIG. 10B. ddPCR quantification of HSV genomes from latent infected TG (open circles, n=11) or reactivated TG (closed circles, n=11). One-tail unpaired t-test shows a significant 3-fold increase in viral loads from reactivated TG compared with latent TG, p=0.0002. *p<0.05, **p<0.01, ***p<0.001, ****p<0.0001 significantly different from controls. All data are presented as mean values+/−SD. Source data are provided as a Source Data file.

FIGS. 11A through 11C. T7E1 analysis of CRISPR/Cas9 gene editing of latent HSV in vivo. FIG. 11A. Mice latently infected with 2×10⁵ PFU HSV-1(F) for 28 days were injected in the right whisker pad with 10¹² vector genomes (vgs) ssAAV1-sCMV-SaCas9-sgRNAUL54 (n=5 per sgRNAUL54). Analysis was performed at either 28 (n=3 mice per sgRNAUL54) or 56 (n=2 mice per sgRNAUL54) days after AAV injection. FIGS. 11B-11C. Detection of mutations by T7E1 assay in HSV genomes from trigeminal ganglia (TG) DNA of treated mice from the experiment described in FIGS. 5D-5F, and collected at either 11B, 28 or 11C, 56 days after AAV administration. Source data are provided as a Source Data file.

FIGS. 12A through 12I. ddPCR quantification of AAV genomes in ganglia. FIG. 12A. Levels of AAV transduction in trigeminal ganglia (TG) from infected mice injected in the right whisker pad with 10¹² vector genomes (vgs) ssAAV1-sCMV-SaCas9-sgRNAUL54 (n=5 per sgRNAUL54). sgRNAUL54-13 (circles), sgRNAUL54-17 (squares), and sgRNAUL54-26 (triangles). Analysis was performed at either 28 (n=3 mice per sgRNAUL54) or 56 (n=2 mice per sgRNAUL54) days post AAV exposure (see FIGS. 5D-5F). FIG. 12B. Levels of AAV transduction of superior cervical ganglia (SCG) and ipsilateral trigeminal ganglia (TG) of latently infected mice (left) untreated (Controls, circles n=8) or administered by whisker pad injection either 5×10¹¹ vector genomes (vgs) scAAV8-CBh-m5 (squares, n=9), 5×10¹¹ vgs scAAV8-CBh-m8 (triangles, n=8) or 5×10¹¹ vgs of each scAAV8-CBh-m5 and scAAV8-CBh-m8 (diamonds, n=9). Analysis was performed 31 days later (see FIG. 1). FIG. 12C. Levels of AAV transduction of SCG and ipsilateral TG of latently infected mice either (left) untreated (CTRL, circles n=3) or administered 0.5-1×10¹² scAAV-Rh10-CBh-m5 by retroorbital (RO) injection (m5, squares n=6). Analysis was performed 41 days later (see FIGS. 2A-2C). FIG. 12D. Levels of AAV transduction of SCG and ipsilateral TG of latently infected mice either (left) untreated (CTRL, circles n=12) or administered by RO injection 5×10¹¹ vgs scAAV-Rh10-CBh-m5+5×10¹¹ vgs scAAV-Rh10-CBh-m8 (m5+m8, squares, n=12). Analysis was performed 33-35 days later (see FIGS. 2D-2F). FIG. 12E. Levels of AAV transduction of SCG and ipsilateral TG of latently infected mice either (left) untreated (CTRL, circles n=12) or administered by RO injection 1×10¹² vgs scAAV-Rh10-CBh-m4 (m4, n=12). Analysis was performed 33-35 days later (see FIGS. 3A-3C). FIG. 12F. Levels of AAV transduction of SCG and ipsilateral TG of latently infected mice either (left) untreated (CTRL, circles n=10) or administered by RO injection 5×10¹¹ vgs scAAV-Rh10-CBh-m5+5×10¹¹ vgs scAAV-Rh10-CBh-m4 (m5+m4, squares, n=10). Analysis was performed 33-35 days later (see FIGS. 3D-3F). FIG. 12G. Levels of AAV transduction of reactivated SCG and ipsilateral TG of latently infected mice either (left) untreated (CTRL, circles n=12) or administered 5×10¹¹ vgs scAAV-Rh10-CBh-m5+5×10¹¹ vgs scAAV-Rh10-CBh-m8 (m5+m8, squares, n=12) by RO injection (see FIG. 4). FIGS. 12H-12I. Levels of AAV transduction of SCG (12H) and ipsilateral TG (12I) of latently infected mice either left untreated (CTRL, circles n=10) or administered by RO injection either dual sgRNA therapy consisting of 10¹² vgs ssAAVRh10-sCMV-SaCas9-sgRNAUL54-26 and 10¹² vgs ssAAVRh10-sCMV-SaCas9-sgRNAUL30-10 (Cas9 squares, n=10) or meganuclease therapy of 10¹² vgs ssAAVRh10-smCBA-m5-Trex2-mCherry (m5+Trex2 triangles, n=10). Analysis was performed 28/29 days later (see FIG. 6). All data are presented as mean values+/−SD. Source data are provided as a Source Data file.

FIGS. 13A through 13J. Poor efficiency of HSV gene editing by SaCas9 in infected mice. FIG. 13A. Mice were latently infected with 2×10⁵ PFU HSV-1(F) for 28 days, and then injected in the right whisker pad (n=3) with 10¹² vgs SaCas9-expressing ssAAV1 or ssAAV8 under the indicated promoter and sgRNA targeting either the UL54 (UL54-26: sgRNAUL54-26) or UL30 (UL30-1: sgRNAUL30-1 and UL30-10: sgRNA_{UL30}-10) genes (n=3 per group). Analysis was performed at 28 days after AAV exposure. FIGS. 13B-13C, levels of HSV and FIGS. 13D-13E, levels of AAV genomes were quantified by ddPCR in right (ipsilateral) trigeminal ganglia (TGs) from infected mice. FIGS. 13F-13H. Detection of mutations by T7E1 assay in HSV genomes from TG DNA 28 days after injection with 13F, ssAAV1 (1) or ssAAV8 (8) carrying SaCas9 under the indicated promoter and sgRNAUL54-26; 13G, ssAAV8 carrying SaCas9 under the sCMV promoter, along with the indicated sgRNAUL30; 13H, ssAAV1 (1) carrying SaCas9 under the CBh promoter along with sgRNAUL54-26. T7−: T7E1 negative control; T7+: T7E1 positive control, mw: molecular weight marker, bp: base pairs. The gel images were cropped. FIGS. 13I-13J. Mutagenic event detection by NGS analysis of the PCR products used in the T7E1 analysis. The dotted lines mark the levels of background mutation detected at the site targeted by the respective sgRNA in PBS treated animals. All data are presented as mean values+/−SD. Source data are provided as a Source Data file.

FIGS. 14A through 14E. Gene expression patterns that define cluster identity. FIG. 14A. Heatmap of the top 10 most upregulated genes in each cluster. FIGS. 14B-14E. Heatmaps generated by comparing the top 100 most upregulated genes from each cluster of our study to the top 100 most upregulated genes from the cluster of either 14B, our study; 14C, (Nguyen, M. Q., Wu, Y., Bonilla, L. S., von Buchholtz, L. J. & Ryba, N. J. P. Diversity amongst trigeminal neurons revealed by high throughput single cell sequencing. PLOS One 12, e0185543, doi:10.1371/journal.pone.0185543 (2017)); 14D, (Usoskin, D. et al. Unbiased classification of sensory neuron types by large-scale single-cell RNA sequencing. Nat Neurosci 18, 145-153, doi:10.1038/nn.3881 (2015)); 14E, (Li, C. L. et al. Somatosensory neuron types identified by high-coverage single-cell RNA-sequencing and functional heterogeneity. Cell Res 26, 83-102, doi: 10.1038/cr.2015.149 (2016)). The scale indicates the percent of overlapping genes.

FIGS. 15A through 15F. Distribution of HSV and AAV positive cells across clusters. Neurons that express HSV genes (light grey dots) (FIG. 15B) or the transgene delivered via the indicated AAV (grey dots) (FIG. 15C AAV1; FIG. 15D AAV8; FIG. 15E PHP.S; FIG. 15F Rh10) were overlaid onto tSNE plots (FIG. 15A). Note that only one-quarter of the analyzed animals received each AAV serotype, so the actual saturation of neuronal subsets is greater than it appears in this representation. All animals received HSV. Total percentage of transgene positive neurons from superior cervical ganglia (SCG) and trigeminal ganglia (TG) is indicated in the upper left and lower right, respectively. *Note that ¼ of the mice received each of the 4 AAV serotypes, and samples were pooled for library construction and sequencing. Therefore, each serotype could transduce a theoretical maximum of ¼ of the neurons. All mice were infected with HSV.

FIGS. 16A through 16E. Percentage of neurons in each neuronal cluster positive for HSV or the indicated AAV transgene: 16A HSV; 16B AAV1; 16C AAV8; 16D PHP.S; 16E Rh10. trigeminal ganglia-1 (TG-1) to TG-10 clusters (black bars) and superior cervical ganglia-1 (SCG-1) to SCG-5 clusters (grey bars). For AAV expression, percentages were normalized to input as described in Methods.

FIGS. 17A through 17E. Percent difference in the fractional distributions of HSV+ or AAV+ cells within clusters relative to a random distribution. 17A HSV; 17B AAV1; 17C AAV8; 17D PHP.S; 17E Rh10. Trigeminal ganglia-1 (TG-1) to TG-10 clusters (black bars) and superior cervical ganglia-1 (SCG-1) to SCG-5 clusters (grey bars). The following formulas were used to compute the percent difference from random: For HSV+ cells: Percent difference from random=[(Number of HSV+ cells detected in the cluster-expected number of HSV+ cells in the cluster¹)/total number of cells in the cluster]×100. For AAV+ cells: Percent difference from random=[(Number of AAV+ cells detected in the cluster-expected number of AAV+ cells in the cluster¹)/Normalized total number of cells in the cluster²]×100. ¹ The expected number of positive cells in the cluster=number of positive cells within a tissue (TG or SCG)×[total number of cells in the cluster/total number of cells in tissue].² The normalized total number of cells in the cluster=the total number of cells in the cluster×the fraction of cells contributed by mice injected with a given AAV serotype.

FIGS. 18A through 18E. Distribution of HSV and AAV double positive cells across clusters. Cells that express an HSV gene and the transgene delivered via the indicated AAV (FIG. 18B HSV/AAV1⁺; FIG. 18C HSV/AAV8⁺; FIG. 18D HSV/PHP.S⁺; FIG. 18E HSV/Rh10⁺) were overlaid onto the tSNE plot (FIG. 18A). Neurons positive for HSV transcript HSV+ (light grey dots), the transgene transcript of the indicated AAV serotype (grey dots) or both HSV+/AAV+ (diamonds). Only one-quarter of the analyzed animals received each AAV serotype, so the actual saturation of neuronal subsets is greater than it appears in this representation. All animals received HSV. Percentage of HSV positive neurons also positive for AAV transgene in superior cervical ganglia (SCG) and trigeminal ganglia (TG) is indicated in the upper left and lower right, respectively. *Note that ¼ of the mice received each of the 4 AAV serotypes, and samples were pooled for library construction and sequencing. Therefore, each serotype could transduce a theoretical maximum of ¼ of the neurons. All mice were infected with HSV.

FIG. 19. Schematic for JQ1 reactivation experiment. Latent infected mice were divided into three experimental groups to determine the effect of JQ1 on HSV reactivation. As illustrated in the figure, Group 1 and Group 2 were JQ1 groups that received a 50 mg/kg intraperitoneal injection of JQ1. Group 3 was the control group, i.e., no JQ1 injection. Group 1 mice received two injections, the first JQ1 injection at 0 hrs and the second JQ1 injection at 12 hrs. Group 2 mice received one injection at 0 hrs. Group 3 mice received one control injection at 0 hrs. To determine shedding from mucosal surfaces, as an indicator of HSV reactivation, mice were swabbed at 0 hrs (control) and this swab was compared to swabs taken at 24 hours, 48 hours, and 72 hours.

FIGS. 20A through 20C. JQ1 treatment leads to shedding of HSV. Continuing from the protocol described in FIG. 19, FIG. 20A no shedding was detected in the control mice (Group 3). FIG. 20B. Shedding was detected in the JQ1 single dose mice (Group 2), which peaked 2 days following the JQ1 injection. FIG. 20C. Shedding was also detected in the mice that received two doses of JQ1 (Group 1).

FIGS. 21A through 21C. The dual-meganuclease combined with JQ1 reduced HSV-1 load. FIG. 21A. In mice treated with the dual-meganuclease and JQ1 the HSV viral load decreased at least 83% in superior cervical ganglia cells 48 hours following JQ1 treatment; this reduction in HSV load increased to at least 97%96 hours following JQ1 treatment. FIG. 21B. In mice treated with the dual-meganuclease and JQ1 the HSV load decreased at least 97% in trigeminal ganglia cells 96 hours following JQ1 treatment. Unlike superior cervical ganglia cells, no reduction in HSV load was observed in trigeminal ganglia cells 48 hours following JQ1 treatment.

### DETAILED DESCRIPTION

The inventors evaluated gene editing of HSV-1 in a well-established mouse model, using adeno-associated virus (AAV)-delivered meganucleases, as a potentially curative approach to treat latent HSV-1 infection. This disclosure describes that AAV-delivered meganucleases, but not CRISPR/Cas9, mediate highly efficient gene editing of HSV-1, eliminating over 90% of latent virus from superior cervical ganglia. Single-cell RNA sequencing demonstrates that both HSV-1 and individual AAV serotypes are non-randomly distributed among neuronal subsets in ganglia, implying that improved delivery to all neuronal subsets may lead to even more complete elimination of HSV-1. As described in more detail below in Example 1, delivery of meganucleases using a triple AAV serotype combination resulted in the greatest decrease in ganglionic HSV-1 loads. The levels of HSV-1 elimination observed in these studies, if translated to humans, would likely significantly reduce HSV-1 reactivation, shedding, and lesions. Further optimization of meganuclease delivery and activity is likely possible and may offer a pathway to a cure for HSV-1 infection.

### Herpes Simplex Virus

Herpes simplex virus (HSV) comprises at least HSV type 1. As used herein HSV-1 and HSV are used interchangeable to refer to HSV type 1 (HSV-1). HSV-1 belongs to the Herpesviridae family of DNA viruses that cause infections in humans. HSV-1, once acquired remains with the host for life, and typically remains latent in the form of stable dsDNA episome in the nuclei of sensory neurons. HSV-1 is a highly adapted human pathogen with a rapid lytic replication cycle and also exhibits the ability to invade sensory neurons without showing any cytopathology. Latent infections are subject to reactivation whereby infectious virus can be recovered in peripheral tissue enervated by the latently infected neurons following a specific physiological stress. A major factor in these switches from lytic to latent infection and back involves changes in transcription patterns, mainly as a result of the interaction between viral promoters, the viral genome and cellular transcriptional machinery.

The primary infection site for HSV-1 is at the mucosal surfaces. In some embodiments, HSV-1 can access sensory nerve endings and through retrograde transport migrate from the site of infection to the trigeminal ganglion (TG) and superior cervical ganglion (SCG). There, HSV-1 can infect the TG and SCG, and the TG and SCG remain the site of latency until HSV-1 is reactivated by, among other things stress, where HSV-1 migrates from the TG or SCG through retrograde transport to the primary site of infection.

The HSV-1 genome is a linear, double stranded DNA duplex 152,261 base pairs (bp) in length, and with a base composition of 68% G+C which circularizes upon infection. The HSV-1 genome is divided into six important regions. One, the ends of the linear molecules, the “a” sequences: these are important in both circularization of the viral DNA, and in packaging the DNA in the virion. Two, the 9,000 bp long repeat (RL), which encode both an important immediate early regulatory protein (aO) and the promoter of most of the “gene” for the latency associated transcript (LAT). Three, the long unique region (UL), which is 108,000 bp long, encodes at least 56 distinct proteins; it contains, for example, genes for the DNA replication enzymes and the capsid proteins. Four, the 6,600 bp short repeats (Rs) encode the very important “a” immediate early protein; this is a very powerful transcriptional activator which acts along with aO ICP0 and a27 (ICP27/UL54) (in the UL) to stimulate the infected cell for all viral gene expression that leads to viral DNA replication. Five, the origins of replication: the OHL is in the middle of the U_{L} region; the oris is in t e Rs and thus, is present in two copies. All sets of ori's operate during infection to give a very complicated replication complex, very similar to that seen in the replication of phage T4. Six, the 13,000 bp unique short region (Us) encodes 12 ORFs, a number of which are glycoproteins important in viral host range and response to host defense.

The virus encodes nearly 100 transcripts and more than 70 open translational reading frames (ORFs). Most ORFs are expressed by a single transcript. About 40 genes are considered as essential for virus replication in culture, including U_{L}19, U_{L}30, and U_{L}54. U_{L}19 is expressed in the late stages of the infection cycle and codes for the major capsid protein, VPR. U230 is expressed in the early stages of the infection cycle and codes for the catalytic subunit of the viral DNA polymerase. U_{L}54 is expressed in the intermediate stages of the infection cycle and codes for the immediate early regulatory protein ICP27. ICP0 is expressed and functions at the earliest stages of the productive infection cycle and is important to initiate early transcription and replication.

### Meganucleases

Meganucleases are essentially represented by homing endonucleases. Homing Endonucleases (HEs) are a widespread family of natural meganucleases including hundreds of proteins families (Chevalier, B. S. and B. L. Stoddard, Nucleic Acids Res., 2001, 29, 3757-3774). These proteins are encoded by mobile genetic elements which propagate by a process called “homing”: the endonuclease cleaves a cognate allele from which the mobile element is absent, thereby stimulating a homologous recombination event that duplicates the mobile DNA into the recipient locus.

As used here, “meganuclease” is a double-stranded endonuclease having a large polynucleotide recognition site, at least 12 bp, preferably from 12 by to 60 bp. Meganucleases are also called rare-cutting or very rare-cutting endonucleases. In some embodiments, meganucleases can be either monomeric or dimeric. In some embodiments, the meganuclease can be any natural meganuclease such as a homing endonuclease. In some embodiments, the meganuclease can be any artificial or man-made meganuclease endowed with such high specificity, either derived from homing endonucleases of group I introns and inteins, or other proteins such as Zinc-Finger proteins or group II intron proteins, or compounds such as nucleic acid fused with chemical compounds.

The detailed three-dimensional structures of several homing endonucleases are known, namely I-Dmo I, PI-Sce I, PI-Pfu I, I-Cre I, I-Ppo I, and a hybrid homing endonuclease I-Dmo I/I-Cre I called E-Dre I (Chevalier et al., 2001, Nat Struct Biol, 8, 312-316; Dunn et al., 1997, Cell, 89, 555-564; Heath et al., 1997, Nat Struct Biol, 4, 468-476; Hu et al., 2000, J Biol Chem, 275, 2705-2712; Ichiyanagi et al., 2000, J Mol Biol, 300, 889-901; Jurica et al., 1998, Mol Cell, 2, 469-476; Poland et al., 2000, J Biol Chem, 275, 16408-16413; Silva et al., 1999, J Mol Biol, 286, 1123-1136; Chevalier et al., 2002, Molecular Cell, 10, 895-905).

The LAGLIDADG family is the largest family of proteins clustered by their most general conserved sequence motif: one or two copies of a twelve-residue sequence: the di-dodecapeptide, also called LAGLIDADG motif. Homing endonucleases with one dodecapeptide (D) are around 20 kDa in molecular mass and act as homodimer. Those with two copies (DD) range from 25 kDa (230 AA) to 50 kDa (HO, 545 AA) with 70 to 150 residues between each motif and act as monomer. Cleavage is inside the recognition site, leaving 4 nt staggered cut with 3′OH overhangs. I-Ceu I, and I-Cre I illustrate the homodimeric homing endonucleases with one Dodecapeptide motif (mono-dodecapeptide). The initial LAGLIDADG homing endonuclease can be selected from the group comprising: I-Dmo I, I-Cre I, PI-Sce I, and PI-Pfu I.

### Bromodomain and Extra Terminal Domain (BET) Proteins

Bromodomain and extra-terminal (BET) proteins are a group of epigenetic readers that play a pivotal role in the epigenetic process, and indeed may control expression of genes involved in cell growth and oncogenesis. The posttranslational acetylation of nucleosome histone N-terminal tails represents the fundamental epigenetic mark of open structure chromatin and active gene transcription. Members of the BET protein family feature highly homologous, tandem bromodomains (BD-1 and BD-2) that recognize and bind these acetylated lysine histone tails. The BET proteins then act as scaffolds that recruit transcription factors and chromatin organizers which are required for transcription. For example, via a set of hydrogen-bonding interactions between highly conserved asparagine and tyrosine residues and the acetylated lysine, the BET bromodomains link chromatin to the CDK9-containing complex P-TEFb, which phosphorylates the large subunit of RNA Polymerase II and facilitates pause release and transcript elongation.

The terms “Bromodomain and Extra Terminal Domain” or “BET” as used herein, unless otherwise specified, includes any natural and/or artificial BET from any source. The term “BET” refers to members of the BET family, including BRD2, BRD3, BRD4 and BRDT. Interfering with BET protein interactions via bromodomain inhibition results in modulation of transcriptional programs that are often associated with diseases characterized by dysregulation of cell cycle control, inflammatory cytokine expression, viral transcription, hematopoietic differentiation, insulin transcription, and adipogenesis.

In accordance with the foregoing, in one aspect the disclosure provides methods and compositions for reducing or eliminating latent herpes complex virus type 1 (HSV-1) reactivation in a cell.

In one aspect, the method for reducing or eliminating latent HSV-1 reactivation in a cell can comprise delivering to an HSV-1-infected cell one or more viral vectors comprising one or more sequences encoding one or more HSV-1-specific meganucleases.

In another aspect, the composition for reducing or eliminating latent HSV-1 reactivation in a cell can comprise one or more viral vectors comprising one or more sequences encoding one or more HSV-1-specific meganucleases.

As used herein, “viral vectors” refer to the use of adeno-associated virus vectors (AAV) or any viral vector engineered from an AVV to deliver one or more HSV-1-specific meganuclease to the desired target. In some embodiments, the engineered AAV can comprise a self-complementary adeno-associated virus (scAAV). In some embodiments, the engineered AAV can comprise a single-stranded adeno-associated virus (ssAAV). The viral vectors, e.g., AAVs, scAAVs, ssAAVs, and the like, comprising one or more sequences encoding one or more HSV-1-specific meganucleases were generated according to the method of Choi et al., (Choi, V. W., Asokan, A., Haberman, R. A. & Samulski, R. J. Production of recombinant adeno-associated viral vectors for in vitro and in vivo use. Curr Protoc Mol Biol Chapter 16, Unit 16 25, doi:10.1002/0471142727.mb1625s78 (2007)) the contents of which are herein incorporated by reference.

In some embodiments, the one or more viral vectors is an AAV. In some embodiments, the AAV can comprise any serotype well known to those with ordinary skill in the art. In some embodiments, the one or more AAVs can comprise AAV1, AAV2, AAV3, AAV4, AAV5, AAV6, AAV7, AAV 8, AAV9, AAV10, AAV11, AAV12, AAV-Rh8, AAV-rh10 serotype adeno-associated virus, or a combination thereof. In some embodiments, the one or more AAVs can comprise AAV-Rh10, AAV8, AAV1 serotype adeno-associated virus, or a combination thereof. In still other embodiments, the one or more AAVs is AAV-Rh10 and/or AAV8 serotype adeno-associated virus.

In some embodiments, the one or more viral vectors is an scAAV. In some embodiments, the scAAV can comprise any serotype well known to those with ordinary skill in the art. In some embodiments, the one or more scAAVs can comprise AAV1, AAV2, AAV3, AAV4, AAV5, AAV6, AAV7, AAV 8, AAV9, AAV10, AAV11, AAV12, AAV-Rh8, AAV-rh10 serotype adeno-associated virus, or a combination thereof. In some embodiments, the one or more scAAVs can comprise AAV-Rh10, AAV8, AAV1 serotype adeno-associated virus, or a combination thereof. In still other embodiments, the one or more scAAVs is AAV-Rh10 and/or AAV8 serotype adeno-associated virus.

In some embodiments, the one or more viral vectors is an ssAAV. In some embodiments, the ssAAV can comprise any serotype well known to those with ordinary skill in the art. In some embodiments, the one or more ssAAVs can comprise AAV1, AAV2, AAV3, AAV4, AAV5, AAV6, AAV7, AAV 8, AAV9, AAV10, AAV11, AAV12, AAV-Rh8, AAV-rh 10 serotype adeno-associated virus, or a combination thereof. In some embodiments, the one or more ssAAVs can comprise AAV-Rh10, AAV8, AAV1 serotype adeno-associated virus, or a combination thereof. In still other embodiments, the one or more ssAAVs is AAV-Rh10 and/or AAV8 serotype adeno-associated virus.

In some embodiments, the one or more HSV-1-specific meganucleases can be configured to induce one or more DNA double strand breaks (DSB). DNA DSBs are created in HSV-1 genomes upon expression of homing endonucleases that target specific sequences in essential HSV-1 genes. DSBs are repaired by non-homologous end joining which is error prone so that continual cleavage of HSV-1 target sites leads to disruption/mutation of HSV-1 genes. As used herein, the phrase “configured to induce one or more DNA DSBs” refers to the use of meganucleases that target specific HSV-1 target site(s) to cause DSBs. As used herein, “target sequence” or “target site” refers to a nucleic acid sequence within the viral genome that comprises a sequence to which the specific meganuclease targets resulting in gene editing of HSV-1 target sites.

In some embodiments, the one or more HSV-1-specific meganucleases are derived from the I-Crel enzyme. In some embodiments, HSV-1-specific meganucleases can be configured to induce one or more DNA DSBs in any HSV-1 gene that is well known to one of ordinary skill in the art. For example, in some embodiments, the one or more HSV-1-specific meganucleases is a meganuclease that can be configured to target U_{L}19, U_{L}30, U_{L}19, U_{L}54, ICP0, and the like. In some embodiments, the one or more HSV-1-specific meganucleases is a meganuclease that can be configured to target any combination of U_{L}19, U_{L}30, ICP0, UL54, and/or any HSV-1 gene well known to one of ordinary skill in the art.

In still other embodiments, the HSV-1-specific meganuclease is HSV1m5 that targets a 24 bp sequence in U_{L}19. In some embodiments, the HSV-1-specific meganuclease is HSV1m8 that targets a 24 bp sequence in U_{L}30. In some embodiments, the HSV-1-specific meganuclease is HSV1m4 that targets the duplicated gene ICP0. In still other embodiments, the HSV-1 specific meganuclease is any combination of HSV1m5, HSV1m8, and/or HSV1m4.

In some embodiments, the one or more meganuclease can comprise a sequence as set forth in SEQ ID NOs: 1-3. In some embodiments, the one or more meganuclease is a meganuclease that can be configured to target one or more sequences as set forth in SEQ ID NOs: 4-6.

In some embodiments, the method can comprise delivering one viral vector (e.g., AAV, scAAV, ssAAV, and the like) comprising two or more sequences encoding one or more HSV-1-specific meganucleases. In some embodiments, the method can comprise delivering two viral vectors (e.g., AAV, scAAV, ssAAV, and the like) each comprising one or more sequences encoding one or more HSV-1-specific meganucleases. In still other embodiments, the method can comprise delivering two different viral vectors (e.g., AAV, scAAV, ssAAV, and the like) each comprising a sequence encoding an HSV-1-specific meganuclease, wherein the sequences are the same or different. In some embodiments, the two different viral vectors can comprise delivering, for example, a first scAAV and a second scAAV, i.e., the same type of viral vector. In some embodiments, the different viral vectors can be selected from any combination, for example AAV, scAAV, ssAAV, and the like, i.e, two different types of viral vectors. As used herein, “delivering” refers to administering the one or more viral vectors comprising one or more sequences encoding one or more HSV-1-specific meganuclease into a subject by a method or route which results in at least partial inoculation of the subject at the desired site.

In some embodiments, the one or more viral vectors can be delivered into the cell according to methods generally well known to one of ordinary skill in the art which are appropriate for the particular viral vector and cell type. In some embodiments, the viral vector is delivered to the subject via intravenous injection, subcutaneous injection, intramuscular injection, autologous cell transfer, or allogeneic cell transfer. In still other embodiments, the viral vector is combined with one or more pharmaceutically acceptable carrier for administration. The pharmaceutically acceptable carriers can include those well known to one of ordinary skill in the art and appropriate for the particular viral vector and cell type.

In still other embodiments, an “effective amount” of the viral vector is delivered to edit HSV-1 genome. As used herein, the term “effective” refers to any amount that induces a desired response while not inducing significant toxicity in the subject, e.g., edit the specific HSV-1 genome.

In some embodiments, the cell can be in a mammalian subject. In some embodiments, the mammalian subject can be human. In still other embodiments, the cell can be a superior cervical ganglia (SCG) cell. In still other embodiments, the cell can be a trigeminal ganglia (TG) cell. In still other embodiments, the cell can be a combination of a SCG cell and/or a TG cell.

In some embodiments, the method further comprises administering to the HSV-1-infected cell a bromodomain and extra-terminal (BET) protein inhibitor. As used herein, “BET inhibitor” refers to a compound that binds to BET and inhibits and/or reduces the biological activity of BET. In some embodiments, the BET inhibitor substantially or completely inhibits the biological activity of BET. In some embodiments, the biological activity is binding of BET to chromatin (e.g., histones associated with DNA) and/or another acetylated protein. In some embodiments, the BET inhibitor can inhibit one or more of BRD2, BRD3, BRD4, and BRDT. In some embodiments, the BET protein inhibitor can be selected from any of those BET protein inhibitors well known to one of ordinary skill in the art. In still other embodiments, the BET protein inhibitor can be selected from the group consisting of JQ1, birabresib, molibresib, apabetalone, ZEN-3694, BMS-986158, INC-B057643, and the like.

As used herein, the BET protein inhibitor JQ1, also known as (+)-JQ1 has the following chemical name: (tert-butyl (S)-2-(4-(4-chlorophenyl)-2,3,9-trimethyl-6H-thieno[3,2-f][1,2,4]triazolo[4,3-a][1,4]diazepin-6-yl)acetate).

As used herein, birabresib, also known as OTX015 and MK-8628 has the following chemical name: ((S)-2-(4-(4-chlorophenyl)-2,3,9-trimethyl-6H-thieno[3,2-f][1,2,4]triazolo[4,3-a][1,4]diazepin-6-yl)-N-(4-hydroxyphenyl)acetamide).

As used herein, molibresib, also known as GSK525762, GSK525762A, and I-BET762 has the following chemical name: ((S)-2-(6-(4-chlorophenyl)-8-methoxy-1-methyl-4H-benzo[f][1,2,4]triazolo[4,3-a][1,4]diazepin-4-yl)-N-ethylacetamide).

As used herein, apabetalone also known as RVX-208 and RVX000222 has the following chemical name: (2-(4-(2-hydroxyethoxy)-3,5-dimethylphenyl)-5,7-dimethoxyquinazolin-4(3H)-one).

As used herein, BMS-986158 has the following chemical name: ((S)-2-(3-(1,4-dimethyl-1H-1,2,3-triazol-5-yl)-5-(phenyl(tetrahydro-2H-pyran-4-yl)methyl)-5H-pyrido[3,2-b]indol-7-yl)propan-2-ol).

As used herein, INC-B057643 has the following chemical name: (2,2,4-trimethyl-8-(6-methyl-7-oxo-6,7-dihydro-1H-pyrrolo[2,3-c]pyridin-4-yl)-6-(methylsulfonyl)-2H-benzo[b][1,4]oxazin-3(4H)-one).

In some embodiments, the method can comprise administering the BET protein inhibitor to the HSV-1-infected cell before delivering to the HSV-1-infected cell one or more viral vectors (e.g., AAV, scAAV, ssAAV, and the like) comprising one or more sequences encoding one or more HSV-1-specific meganucleases. In some embodiments, the method can comprise administering the BET protein inhibitor to the HSV-1-infected cell after delivering to the HSV-1-infected cell one or more viral vectors (e.g., AAV, scAAV, ssAAV, and the like) comprising one or more sequences encoding one or more HSV-1-specific meganucleases. In still other embodiments, the method can comprise administering the BET protein inhibitor to the HSV-1-infected cell concomitant with delivering to the HSV-1-infected cell one or more viral vectors (e.g., AAV, scAAV, ssAAV, and the like) comprising one or more sequences encoding one or more HSV-1-specific meganucleases.

In some embodiments, the BET protein inhibitor can be administered at a dose sufficient to provide an “effective concentration” of the BET protein inhibitor to the HSV-1-infected cell. As used herein, “effective” refers to the amount of a BET protein inhibitor that reduces the biological activity of BET, and thus, achieves the desired response without inducing significant toxicity in the subject. In some embodiments, the desired response is induction of viral shedding. In some embodiments, the dose of BET protein inhibitor is administered in mg/kg. In other embodiments, the dose of BET protein inhibitor is administered as a quantity to achieve a particular concentration within the HSV-1-infected cell. Determination of the effective amount of the dose and the type of dose (e.g., a dose based on body weight or a dose to achieve a particular concentration with the HSV-1-infected cell) is well within the capability of those skilled in the art. In some embodiments, the dose can be at least 100 mg/kg. In some embodiments, the dose can be at least 25 mg/kg. In some embodiments, the dose can be at least 50 mg/kg. In some embodiments, the dose can be at least 75 mg/kg. In other embodiments, the dose is administered to achieve a concentration of at least 10 μM in the HSV-1-infected cell. In other embodiments, the dose is administered to achieve a concentration of at least 1 μM in the HSV-1-infected cell. In other embodiments, the dose is administered to achieve a concentration of at least 2 μM in the HSV-1-infected cell. In other embodiments, the dose is administered to achieve a concentration of at least 4 μM in the HSV-1-infected cell. In other embodiments, the dose is administered to achieve a concentration of at least 6 μM in the HSV-1-infected cell. In other embodiments, the dose is administered to achieve a concentration of at least 8 μM in the HSV-1-infected cell.

In some embodiments, the method can reduce HSV-1 load at least 97% in SCG cells at least 96 hours following administration of the BET protein inhibitor. In some embodiments, the method can reduce HSV-1 load at least 83% in SCG cells at least 48 hours following administration of the BET protein inhibitor. In still other embodiments, the method can reduce HSV-1 load at least 97% in TG cells 96 hours following administration of the BET protein inhibitor.

In some embodiments, the composition can reduce HSV-1 load at least 97% in SCG cells 96 hours following administration of the BET protein inhibitor. In some embodiments, the composition can reduce HSV-1 load at least 83% in SCG cells 48 hours following administration of the BET protein inhibitor. In still other embodiments, the composition can reduce HSV-1 load at least 97% in TG cells 96 hours following administration of the BET protein inhibitor.

### Additional Definitions

Unless specifically defined herein, all terms used herein have the same meaning as they would to one skilled in the art of the present invention.

The use of the term “or” in the claims is used to mean “and/or” unless explicitly indicated to refer to alternatives only or the alternatives are mutually exclusive, although the disclosure supports a definition that refers to only alternatives and “and/or.”

Following long-standing patent law, the words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like, are to be construed in an inclusive sense as opposed to an exclusive or exhaustive sense; that is to indicate, in the sense of “including, but not limited to.” Words using the singular or plural number also include the plural and singular number, respectively. Additionally, the words “herein,” “above,” and “below,” and words of similar import, when used in this application, shall refer to this application as a whole and not to any particular portions of the application. The word “about” indicates a number within range of minor variation above or below the stated reference number. For example, “about” can refer to a number within a range of 10%, 9%, 8%, 7%, 6%, 5%, 4%, 3%, 2%, or 1% above or below the indicated reference number.

The term “subject” herein refers to a mammal being assessed for reducing or eliminating latent HSV-1 reactivation. In certain embodiments, the mammal is a human. The term “subject” encompasses, without limitation, individuals having HSV-1. In some embodiments, the subject is one who is diagnosed and currently being treated for, or seeking treatment, monitoring, adjustment or modification of an existing therapeutic treatment, or is at a risk of developing a HSV-1 infection. In one embodiment, the HSV-1 infection is HSV-1-1 infection.

The term “HSV-1 infection” refers to the undesired proliferation or presence of invasion of HSV-1 in a host organism. In some embodiments, the infection can be caused by actively replicating lytic HSV-1 and can be referred to as lytic infection. Such an infection is usually symptomatic. In some embodiments, the infection can be caused by quiescent or latent HSV-1 and can be referred to as latent HSV-1 infection. Such an infection is usually asymptomatic. A latent viral infection can reactivate to become a lytic viral infection or recurrent HSV-1 infection and can result in recurrence of active symptomatic HSV-1 related disease.

As used herein, the term “protein” refers to designate a series of amino acid residues connected to each other by peptide bonds between the alpha-amino and carboxy groups of adjacent residues. The term “protein” can also refer to a polymer of amino acids, including modified amino acids (e.g., phosphorylated, glycated, glycosylated, etc.) and amino acid analogs, regardless of its size or function. The term “protein” can also be used to refer to a gene product and fragments thereof.

As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used here, the term “pharmaceutically acceptable carrier” means a pharmaceutically-acceptable material, composition or vehicle, such as a liquid or solid filler, diluent, excipient, manufacturing aid or solvent encapsulating material necessary or used in formulating an active ingredient or agent for delivery to a subject. Each carrier must be “acceptable” in the sense of being compatible with the other ingredients of the formulation and not injurious to the patient.

The terms, “decrease”, “reduce”, “lower”, “eliminate”, or “inhibit” are all used herein generally to mean a decrease by a statistically significant amount. For example, “decrease”, “reduce”, “lower”, or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level or non-detectable level as compared to a reference level), or any decrease between 10-100% as compared to a reference level. “Eliminate” means complete removal, i.e., detection is not possible.

While illustrative embodiments have been illustrated and described, it will be appreciated that various changes can be made therein without departing from the spirit and scope of the invention. Publications cited herein and the subject matter for which they are cited are hereby specifically incorporated by reference in their entireties.

### EXAMPLES

The following examples are set forth so as to provide those of ordinary skill in the art with a complete disclosure and description of how to make and use the present invention and are not intended to limit the scope of what the inventors regard as their invention nor are they intended to represent that the experiments below are all or the only experiments performed.

### Example 1

This Example describes that AAV-delivered meganucleases, but not CRISPR/Cas9 mediate highly efficient gene editing of HSV, eliminating over 90% of latent virus from superior cervical ganglia. Single-cell RNA sequencing demonstrates that both HSV and individual AAV serotypes are non-randomly distributed among neuronal subsets in ganglia, implying that improved delivery to all neuronal subsets may lead to even more complete elimination of HSV.

In an effort to improve endonuclease-directed gene editing of latent HSV genomes to levels needed for therapeutic benefit, the inventors have evaluated the use of improved self-complementary (sc)AAV vectors, simultaneous targeting of multiple sites within the HSV genome, substitution of CRISPR/Cas9 for meganucleases, and the relative distribution of HSV vs. AAV vectors at the single-cell level. The results described in this Example provide critical insights for the optimization of in vivo gene therapy against HSV, and suggest that meganuclease-mediated gene editing represents a plausible pathway toward HSV cure.

### Results

### Gene Editing Reduces Ganglionic HSV

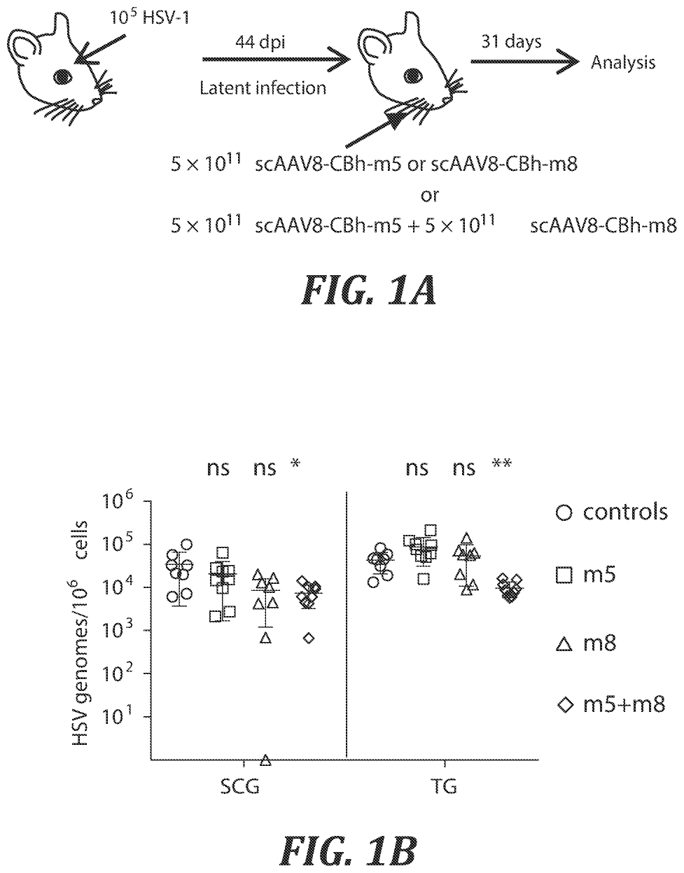

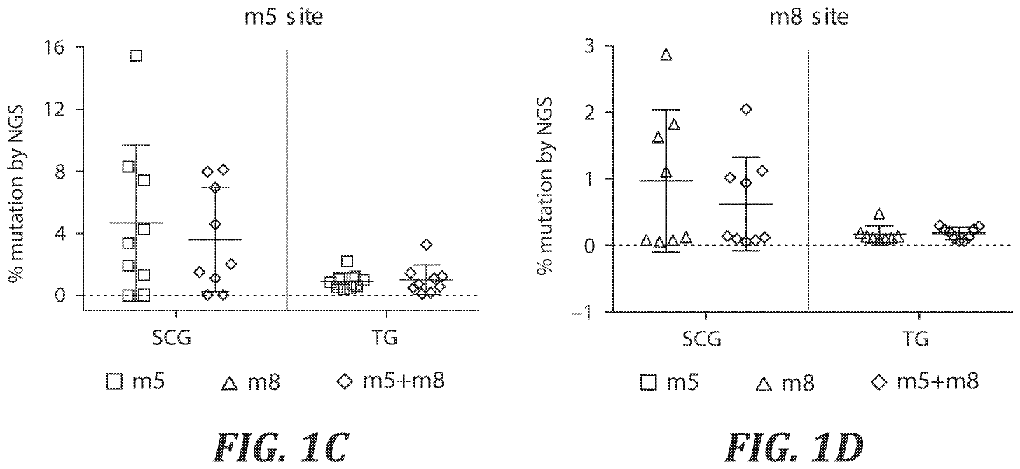

To evaluate the impact of efficient meganuclease-mediated gene editing on latent HSV infection in vivo, a mouse model of HSV ocular infection was used as previously described (Aubert, M. et al. In vivo disruption of latent HSV by designer endonuclease therapy. JCI Insight 1, doi:10.1172/jci.insight.88468 (2016)) (FIG. 1A). The HSV-specific meganucleases HSV1m5 (m5) was used to target U_{L}19 which codes for the major capsid protein VP5; HSV1m8 (m8) was used to target U130 which codes for the catalytic subunit of the viral DNA polymerase, and HSV1m4 (m4) was used to target the duplicated gene ICP0 (Aubert, M. et al. In vivo disruption of latent HSV by designer endonuclease therapy. JCI Insight 1, doi:10.1172/jci.insight.88468 (2016)); Grosse, S. et al. Meganuclease-mediated Inhibition of HSV1 Infection in Cultured Cells. Mol Ther 19, 694-702, doi:10.1038/mt.2010.302 (2011)); Aubert, M. et al. In vitro Inactivation of Latent HSV by Targeted Mutagenesis Using an HSV-specific Homing Endonuclease. Mol Ther Nucleic Acids 3, e146, doi: 10.1038/mtna.2013.75 (2014)). Initially, latently infected mice were treated using an scAAV8 delivery vector, either as single-meganuclease (m5 or m8) or dual-meganuclease (m5+m8) therapy, at the indicated dose (FIG. 1A). A month later, mice were sacrificed, and superior cervical (SCG) and trigeminal (TG) ganglia collected for analysis. In agreement with the inventors' previous results (Aubert, M. et al. In vivo disruption of latent HSV by designer endonuclease therapy. JCI Insight 1, doi:10.1172/jci.insight.88468 (2016)), animals receiving a single meganuclease (either m5 or m8) showed modest levels of gene editing of HSV target sites (means of 4.7% for m5 and 0.97% for m8 in SCG, and 0.92% for m5 and 0.17% for m8 in TG), and neither SCG nor TG showed a detectable reduction in ganglionic HSV load compared with control mice (FIG. 1B). However, when infected mice were treated with dual-meganuclease (m5+m8) therapy, a significant decrease in HSV loads was detected in both SCG and TG, with a mean level of HSV genomes/10⁶ ganglionic cells in SCG of 7.3×10³ in dual-meganuclease-treated mice compared with 3.5×10⁴ in control animals (79% reduction, p=0.018), and in TG of 9.6×10³ in dual meganuclease-treated mice compared with 4.2×10⁴ in control animals (77% reduction, p=0.001) (FIG. 1B). Interestingly, the HSV genomes remaining after dual-meganuclease therapy had mean gene editing levels similar to that of single-nuclease treated mice (3.6% for m5 and 0.63% for m8 in SCG, and 1.0% for m5 and 0.21% for m8 in TG of dual meganuclease-treated mice, FIGS. 1C-1D). In both single- and dual meganuclease-treated mice, the pattern of mutation (mostly small deletions of 1 to 16 bp) seen was consistent with that previously observed in gene editing of HSV (Aubert, M. et al. In vivo disruption of latent HSV by designer endonuclease therapy. JCI Insight 1, doi:10.1172/jci.insight.88468 (2016); Aubert, M. et al. In vitro Inactivation of Latent HSV by Targeted Mutagenesis Using an HSV-specific Homing Endonuclease. Mol Ther Nucleic Acids 3, e146, doi:10.1038/mtna.2013.75 (2014)).

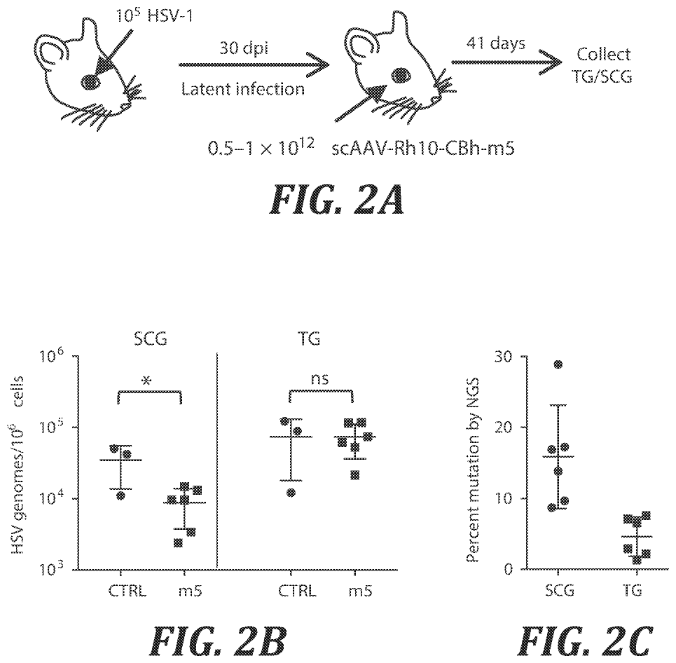

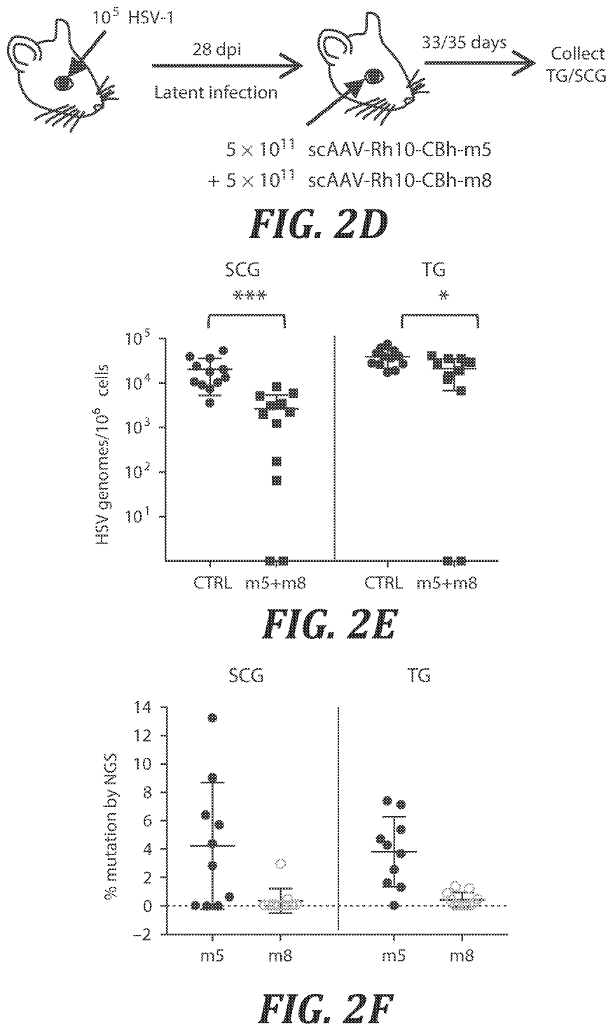

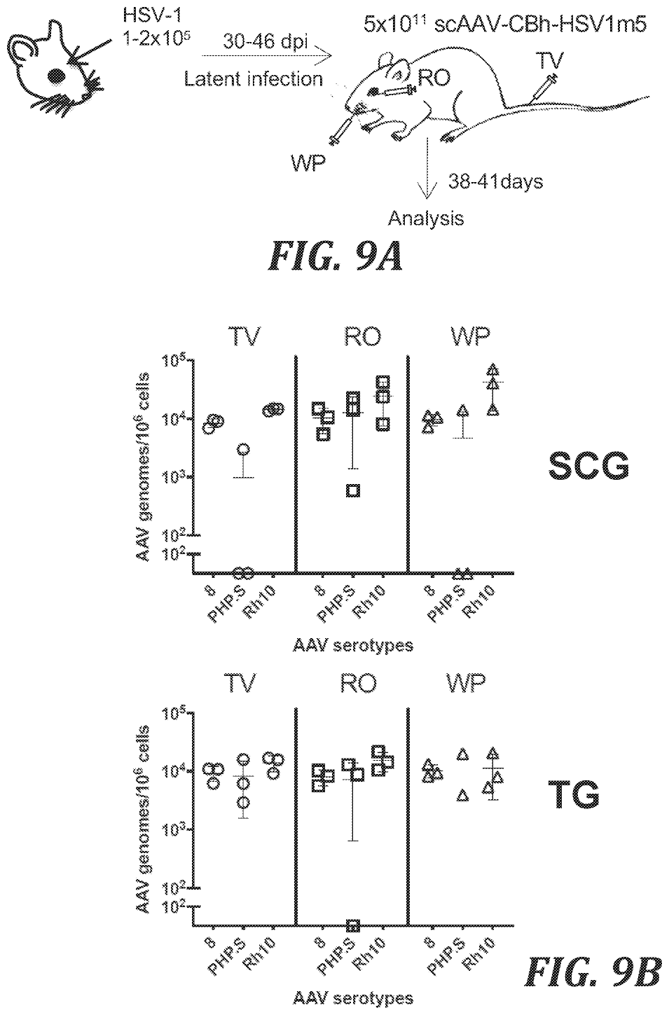

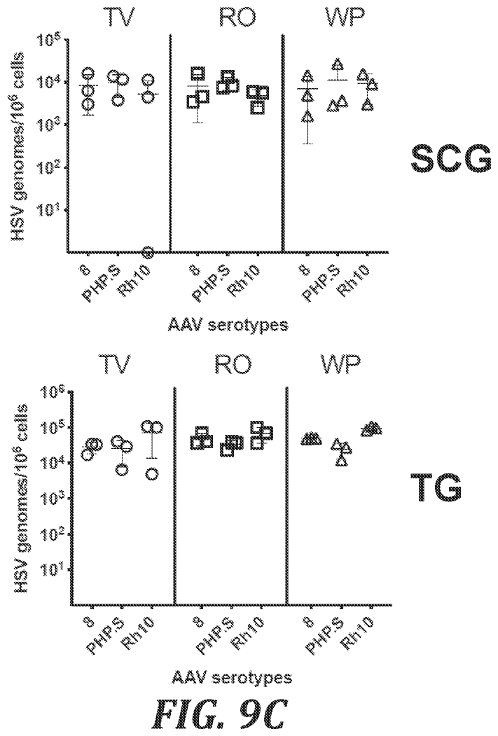

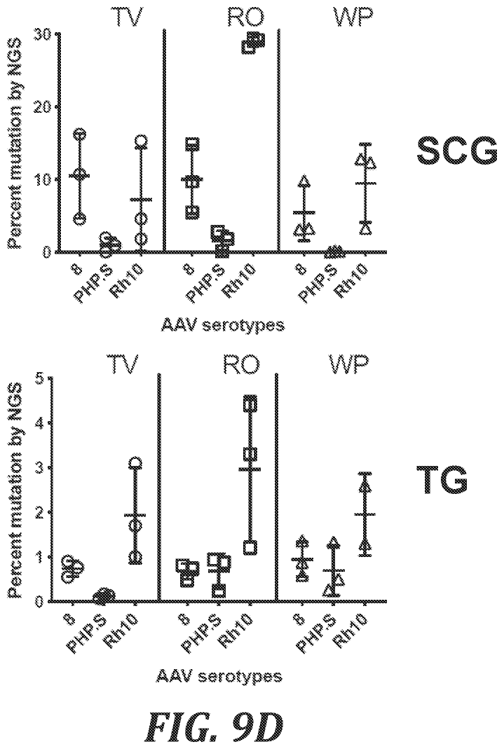

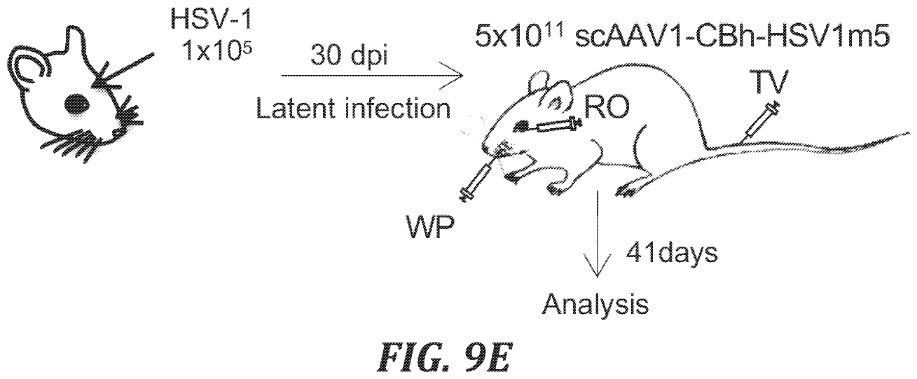

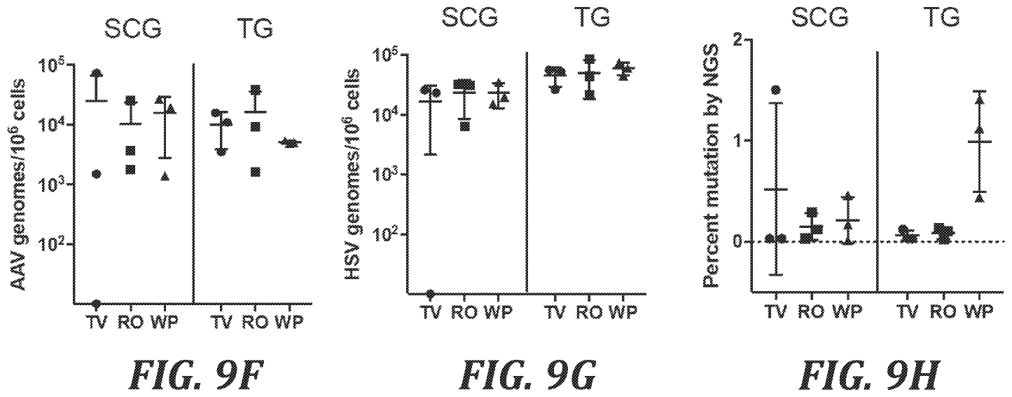

To evaluate the role of AAV serotype in gene editing efficacy, latently infected mice were treated with single-nuclease therapy delivered by a vector derived from the AAV-Rh10 serotype (FIG. 2A). This serotype had demonstrated efficient ganglionic transgene delivery in optimization studies, which showed that the AAV-Rh10 serotype administered via retro-orbital injection led to the highest levels of HSV gene editing in ganglia (FIG. 9). While reduction of viral load in TG did not reach statistical significance as previously seen with dual meganuclease therapy (FIG. 1B), an approximately 65% reduction in mean HSV load in SCG was detected (mean 6×10³ HSV genomes/10⁶ ganglionic cells in treated mice, compared with a mean of 3.7×10⁴ HSV genomes/10⁶ ganglionic cells in control animals, p=0.017) (FIG. 2B). Interestingly, the HSV genomes remaining after single-meganuclease therapy delivered by scAAV-Rh10 (FIG. 2C) showed up to 30% mutagenesis by NGS, a level substantially higher than observed after dual-meganuclease therapy.

Next, dual-meganuclease therapy delivered by AAV serotype Rh10, consisting of 5×10¹¹ vector genomes (vgs) of scAAV-Rh10-CBh-m5 and 5×10¹¹ vgs of scAAV-Rh10-CBh-m8 (FIG. 2D) was evaluated. Dual-meganuclease therapy (m5+m8) delivered by scAAV-Rh10 resulted in a significant decrease of latent HSV genomes in both SCG (mean 2×10⁴ vs. 2.6×10³, 86% reduction, p=0.0006) and TG (mean 3.9×10⁴ vs. 2.1×10⁴, 45% reduction, p=0.01) of treated mice compared with untreated controls (FIG. 2E). Among the virus remaining after therapy, the mean levels of target site mutation were 4.2% for m5 and 0.4% for m8 in SCG, and 3.8% for m5 and 0.4% for m8 in TG (FIG. 2F).

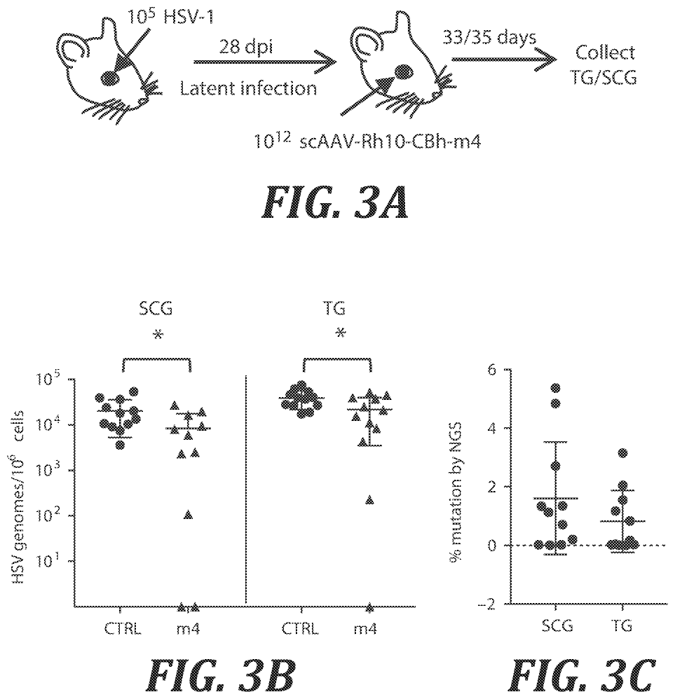

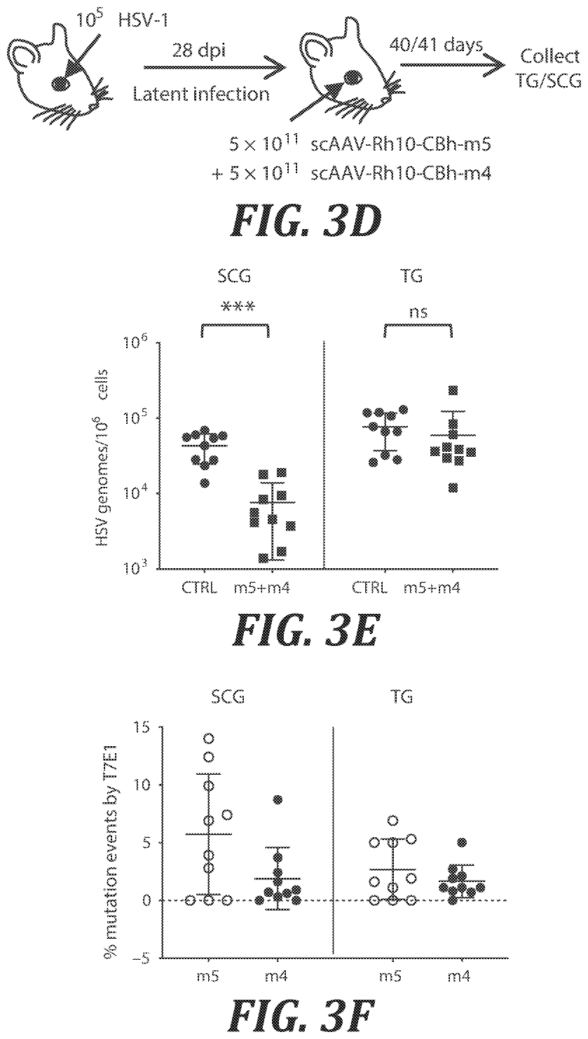

Taken together, these results suggest that while single DNA double strand breaks (DSB) in HSV are typically repaired, often resulting in mutation, the creation of two DNA DSB more commonly leads to degradation and loss of HSV genomes. To evaluate further the importance of dual-compared to single-meganuclease therapy, HSV latently-infected mice were injected with 10¹² vgs of scAAV-Rh10 expressing HSV1m4 (m4), a meganuclease targeting a sequence in the duplicated gene coding for ICP0, which therefore induces two DNA DSB in HSV (FIG. 3A) (Grosse, S. et al. Meganuclease-mediated Inhibition of HSV1 Infection in Cultured Cells. Mol Ther 19, 694-702, doi: 10.1038/mt.2010.302 (2011)). A significant decrease in HSV genomes was detected in both SCG (59% decrease, p=0.03) and TG (45% decrease, p=0.02) of m4-treated mice compared with untreated control animals (FIG. 3B). Consistent with the results from dual m5+m8 meganuclease treated animals, by NGS analysis only 1.6% (SCG) and 0.8% (TG) of target sites in remaining viral DNA were mutated in m4-treated mice (FIG. 3C), again supporting the interpretation that creation of two DNA DSB preferentially leads to degradation of HSV DNA.

To evaluate whether the introduction of more than two DNA DSB would further improve the elimination of HSV genomes by gene editing (FIG. 3D), latently infected mice were administered AAV-Rh10/dual-meganuclease therapy, consisting of 5×10¹¹ vector genomes (vgs) of scAAV-Rh10-CBh-m5 (which targets a single site in HSV) and 5×10¹¹ vgs of scAAV-Rh10-CBh-m4 (which targets two additional sites). While a reduction in HSV viral load was observed in treated animals compared with controls (82.5% in SCG, p=0.00001; 22.5% in TG, p=0.47) (FIG. 3E), along with mutations in the remaining genomes at both the m5 (on average 5.7% in SCG; 2.7% TG) and m4 (on average 1.9% in SCG; 1.7% TG) sites (FIG. 3F), these did not appear to be superior to dual m5+m8 or m4 therapy. The target site mutation frequencies observed were higher for m5 than m4 or m8, which is consistent with a previous report that this enzyme has a higher activity (Grosse, S. et al. Meganuclease-mediated Inhibition of HSV1 Infection in Cultured Cells. Mol Ther 19, 694-702, doi:10.1038/mt.2010.302 (2011)).

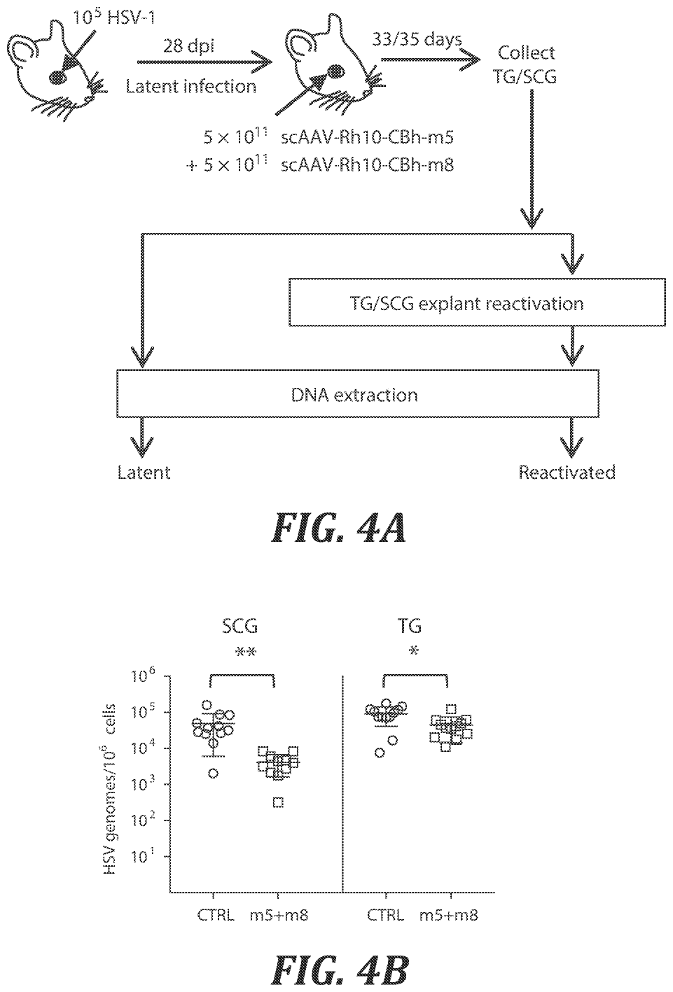

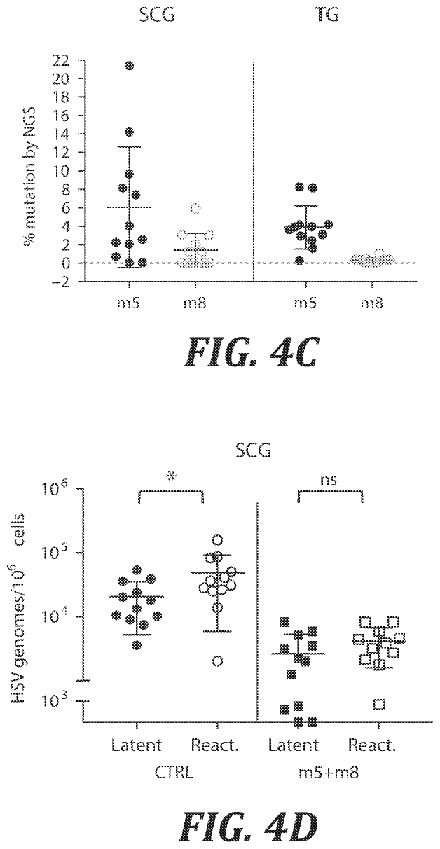

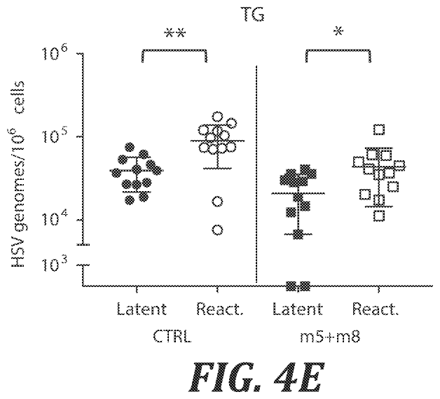

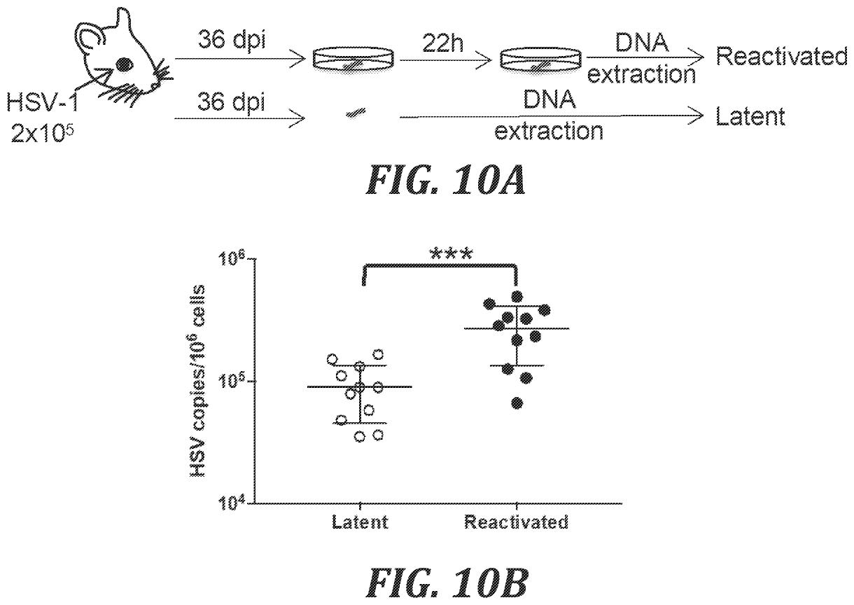

To determine the impact of dual-meganuclease treatment on the ability of HSV to reactivate from ganglia of treated mice, TG and SCG were collected from dual-AAVRh10/meganuclease-(m5+m8) treated and control mice and subjected the tissues to explant reactivation for 24h (FIG. 4A) as previously described (Sawtell, N. M. & Thompson, R. L. Comparison of herpes simplex virus reactivation in ganglia in vivo and in explants demonstrates quantitative and qualitative differences. J Virol 78, 7784-7794, doi:10.1128/JVI.78.14.7784-7794.2004 (2004)). The inventors have shown that ganglionic explant reactivation resulted in an approximate two to three-fold increase in total HSV levels over fresh ganglia, which can be measured by ddPCR (FIGS. 10A-10B). Consistent with the results in FIG. 2, a significant decrease in HSV genomes in reactivated ganglia was detected in dual-meganuclease-treated mice compared with untreated animals in both SCG (90% reduction, mean 4.9×10⁴ vs 4.2×10³, p=0.002), and TG (51% reduction, mean 8.9×10⁴ vs 4.4×10⁴, p=0.01) (FIG. 4B). By NGS, gene editing was detected in 6.0% of residual virus for HSV1m5 and 1.4% for HSV1m8 in SCG, and 3.9% for HSV1m5 and 0.3% for HSV1m8 in TG (FIG. 4C). Strikingly, these decreases in total HSV in reactivated ganglia resulted in a 95% (SCG) and 55% (TG) reduction of de novo produced HSV genomes from dual-treated ganglia compared with untreated control (FIG. 4D-4E and Table 1).

TABLE 1
HSV loads in latent and reactivated ganglia after dual meganuclease therapy

| | | | | | | | | De novo | % |
|---|---|---|---|---|---|---|---|---|---|
| | | | | De novo | Latent | HSV after | Fold | produced | reduction |
| | Latent | HSV after | Fold | produced | HSV in | reactivation | increase | HSV in | in de |
| | HSV in | reactivation | increase | HSV in | m5 + m8 | in m5 + m8 | in HSV in | m5 + m8 | novo |
| | CTRL² | in CTRL² | in HSV in | CTRL | treated² | treated² | m5 + m8 | treated | produce |
| Tissue | (×10³) | (×10³) | CTRL⁴ | (×10³) | (×10³) | (×10³) | treated⁴ | (×10³) | HSV¹ |
| SCG | 20.5 | 48.8 | 2.4 | 28.3 | 2.64² | 4.16² | 1.6 | 1.52 | 94.6 |
| TG | 39.3³ | 89.9³ | 2.3 | 50.6 | 21.0³ | 43.8³ | 2.1 | 22.8 | 54.9 |

¹Percent reduction in de novo produced HSV genomes in dual-meganuclease treated compared with untreated control tissues.
²Mean HSV loads in SCG or TG from untreated control (CTRL) mice with latent (n = 12) or reactivated (n = 12) virus.
³Mean HSV loads in SCG or TG from dual-meganuclease (m5 + m8) treated mice with latent (n = 12) or reactivated (n = 12) virus.
⁴Fold increase in HSV genomes after reactivation in SCG or TG from either untreated control (CTRL) mice or dual-meganuclease (m5 + m8) treated mice.
All data are presented as mean values, and statistical analysis was conducted using one-tail, unpaired t-test. Source data are provided as a Source Data file.

### AAV-Cas9 Mediates Only Weak Gene Editing of HSV In Vivo

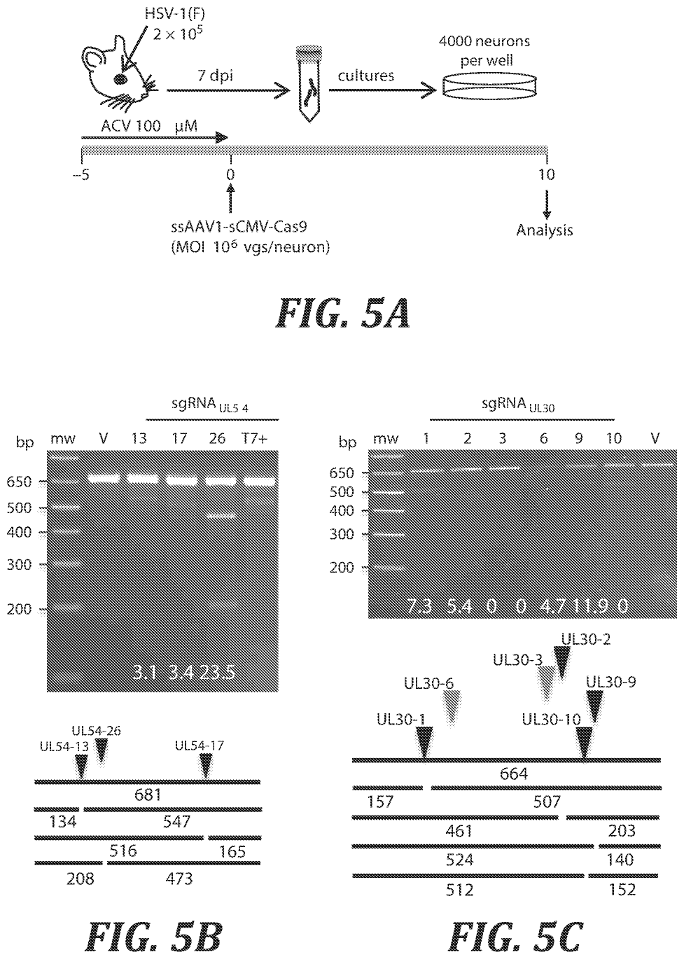

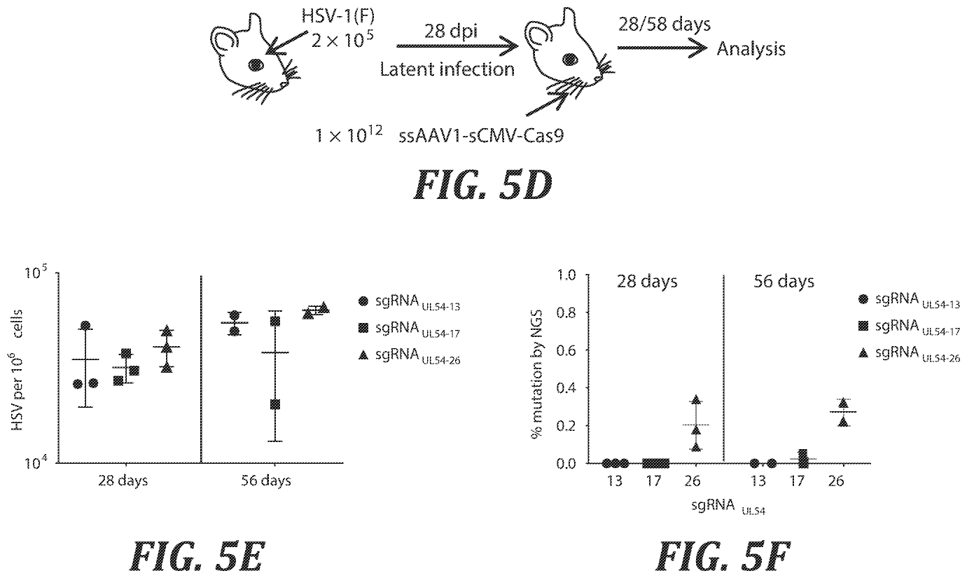

To investigate whether the CRISPR/Cas9 system might allow more efficient gene editing of HSV than meganucleases, several Staphylococcus aureus (Sa)Cas9 sgRNAs were identified that target two essential HSV genes: U_{L}54 encoding the immediate early regulatory protein ICP27 (sgRNA_{UL54} 13, 17, and 26) and U_{L}30 (also the target for meganuclease HSV1m8) coding for the catalytic subunit of the viral DNA polymerase (sgRNA_{UL30} 1 and 10). Due to the large SaCas9 coding sequence (3.1 kb), ssAAV was used for the delivery system. The larger payload capacity of ssAAV allowed both SaCas9 and sgRNA expression cassettes to be on the same AAV construct, ensuring simultaneous delivery of SaCas9 and sgRNA to transduced cells. Several sgRNAs were able to promote high-level Cas9 gene editing of HSV genomes in latently-infected cultured neurons transduced with SaCas9/sgRNA-expressing AAV vectors, as detected by T7 Endonuclease 1 (T7E1) assay (FIG. 5A-5C). By more-sensitive next-generation sequencing (NGS) analysis, the highest level of mutation detected was 49% when SaCas9 was paired with sgRNA_{UL54-26} (Table 2).

TABLE 2
NGS analysis of CRISPR/Cas9 induced mutations in HSV genomes
from infected neuronal cultures treated with SaCas9 and sgRNAUL54-26.

| | | | | SEQ |
|---|---|---|---|---|
| Target site sequence for sgRNA_{UL54-26}¹ | Mutation | Frequency | Percent | ID |
| CATGGCCTTGGCGGTCGATGCGGCCCGAGGATTGCCGGC | Wild type | 3490 | 50.87 | 7 |
| CATGGCCTTGGCGGTCGATGCGGC--GAGGATTGCCGGC | −2 | 1675 | 24.41 | 8 |
| CATGGCCTTGGCGGTCG------CCCGAGGATTGCCGGC | −6 | 186 | 2.71 | 9 |
| CATGGCCTTGGCGGTCGATGCGG----AGGATTGCCGGC | −4 | 143 | 2.08 | 10 |
| CATGGCCTTGGCGGTCGAT----CCCGAGGATTGCCGGC | −4 | 143 | 2.08 | 11 |
| CATGGCCTTGGCGGTCGA----------GGATTGCCGGC | −10 | 122 | 1.78 | 12 |
| CATGGCCTTGGCGGTCGATGC---CCGAGGATTGCCGGC | −3 | 120 | 1.75 | 13 |
| CATGGCCTTGGCGGTCGATGCGGCC-GAGGATTGCCGGC | −1 | 98 | 1.43 | 14 |
| CATGGCCTTGGCGGTCGATGC--CCCGAGGATTGCCGGC | −2 | 89 | 1.30 | 15 |
| CATGGCCTTGGCGGTCGATGCGG--------TTGCCGGC | −8 | 87 | 1.27 | 16 |
| CATGGCCTTGGCGGTCGATGCG-----AGGATTGCCGGC | −5 | 80 | 1.17 | 17 |
| CATGGCCTTGGCGGTCGATGCG-CCCGAGGATTGCCGGC | −1 | 79 | 1.15 | 18 |
| CATGGCCTTGGCGGTCGATGCG---CGAGGATTGCCGGC | −3 | 76 | 1.11 | 19 |
| CATGGCCTTGGCGGTCGATG---------GATTGCCGGC | −9 | 66 | 0.96 | 20 |
| CATGGCCTTGGCGGTCGATGCGG-------ATTGCCGGC | −7 | 54 | 0.79 | 21 |
| CATGGCCTTGGCGGTCGATGCGG-----GGATTGCCGGC | −5 | 54 | 0.79 | 22 |
| CATGGCCTTGGCGG-------------AGGATTGCCGGC | −13 | 40 | 0.58 | 23 |
| CATGGCCTTGGCGGTCGA-----CCCGAGGATTGCCGGC | −5 | 39 | 0.57 | 24 |
| CATGGCCTTGGCGGTCGATGCGG------GATTGCCGGC | −6 | 38 | 0.55 | 25 |
| CATGGCCTTGGCGGTCGA-------CGAGGATTGCCGGC | −7 | 35 | 0.51 | 26 |
| CATGGCCTTGGCGGTCGATGCGG---GAGGATTGCCGGC | −3 | 32 | 0.47 | 27 |
| CATGGCCTTGGCGGTCGATGCGG---------TGCCGGC | −9 | 20 | 0.29 | 28 |
| CATGGCCTTGGCGGTCGATGC----CGAGGATTGCCGGC | −4 | 18 | 0.26 | 29 |
| CATGGCCTTGGCGGTCGATGCGGC-----GATTGCCGGC | −5 | 17 | 0.25 | 30 |
| CATGGCCTTGGCGGTCGAT--------AGGATTGCCGGC | −8 | 17 | 0.25 | 31 |
| CATGGCCTTGGCGGTCGATGC----------TTGCCGGC | −10 | 14 | 0.20 | 32 |
| CATGGCCTTGGCGGTCGAT-----------ATTGCCGGC | −11 | 14 | 0.20 | 33 |
| CATGGCCTTGGCGGTCGA-----------GATTGCCGGC | −11 | 9 | 0.13 | 34 |
| CATGGCCTTGGCGGTC---------------TTGCCGGC | −15 | 3 | 0.04 | 35 |
| CATGGCCTTGGCGGTC--------------ATTGCCGGC | −14 | 3 | 0.04 | 36 |

¹The PAM sequence is bolded.

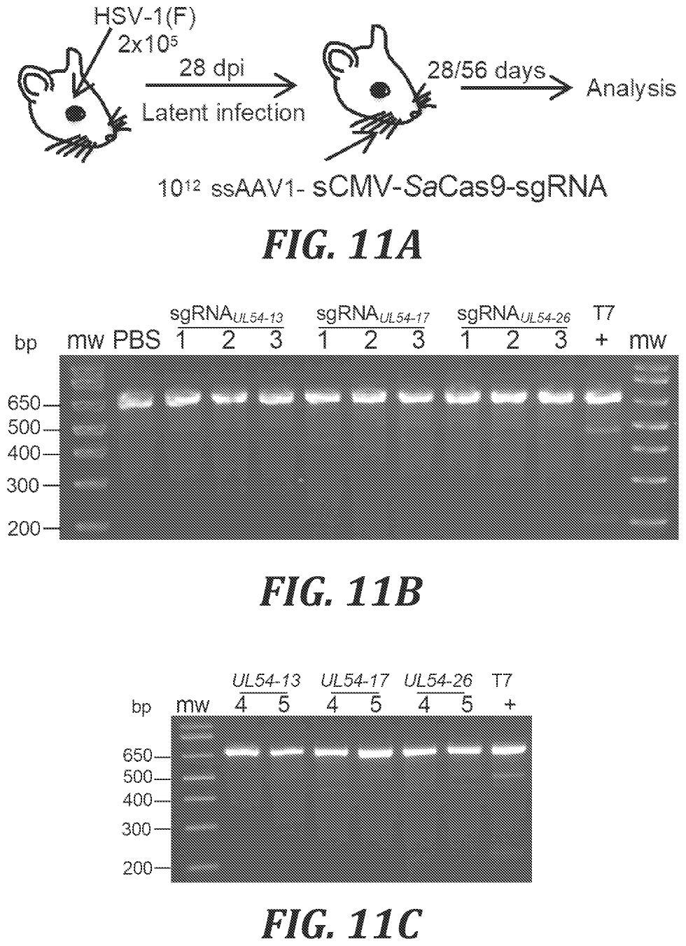

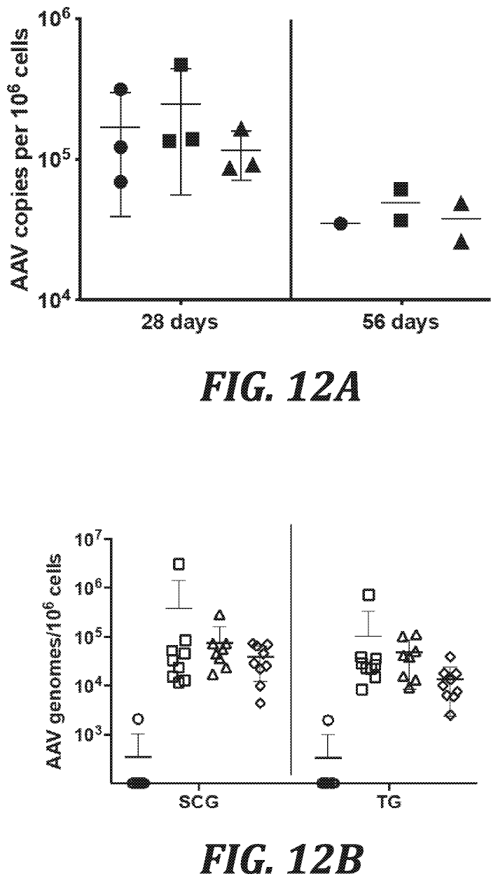

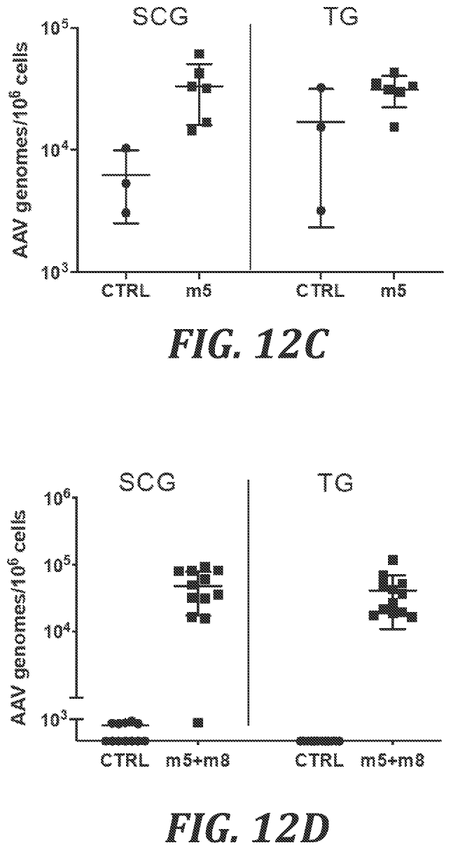

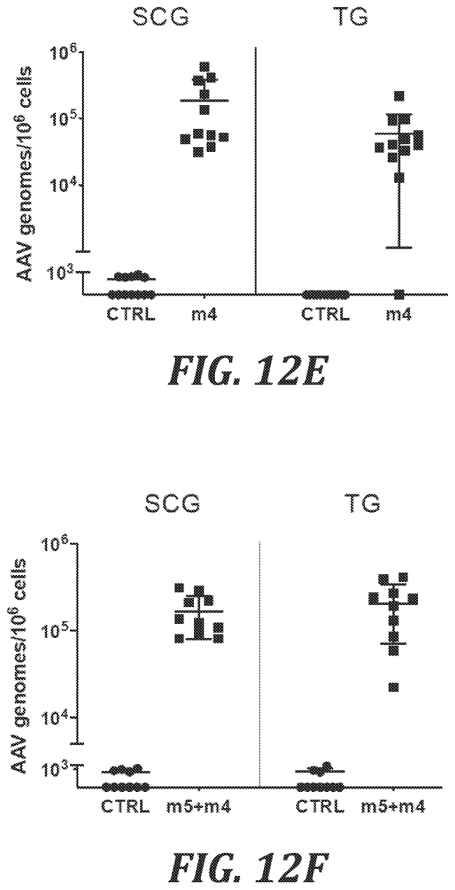

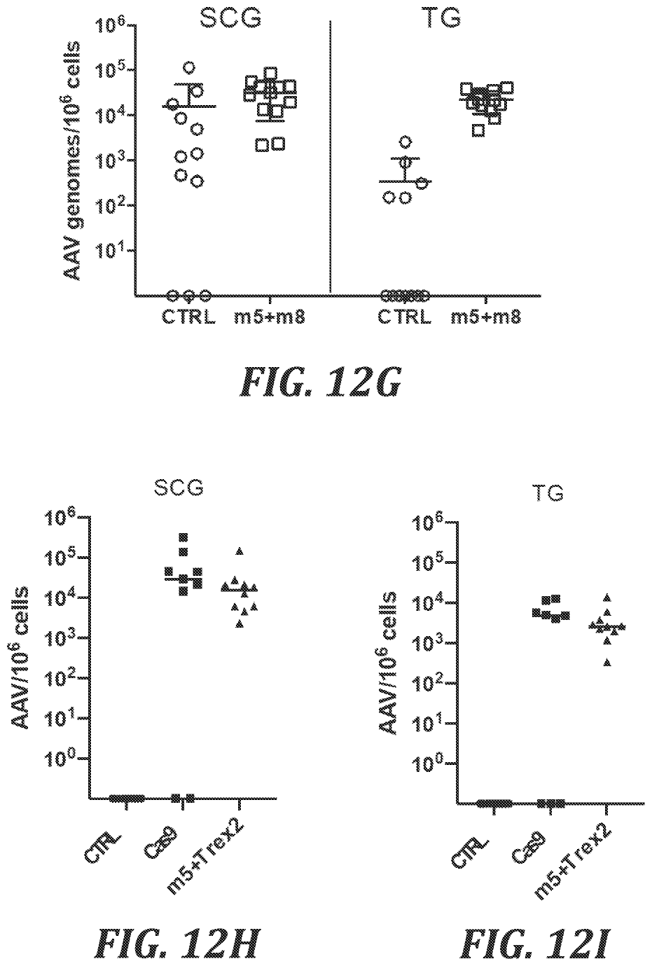

To evaluate the ability of Cas9 to gene edit HSV in vivo, latent HSV infection was established in mice by ocular infection as above (Aubert, M. et al. In vivo disruption of latent HSV by designer endonuclease therapy. JCI Insight 1, doi:10.1172/jci.insight.88468 (2016)). Thirty days after HSV infection, mice were administered 10¹² vgs of ssAAV1-sCMV-SaCas9-sgRNA_{UL54} via whisker pad injection, and TG were collected at 28 and 56 days post-injection for analysis (FIG. 5D). In agreement with results using single-meganuclease therapy, quantification by ddPCR showed similar levels of HSV in the TG of treated and control animals (FIG. 5E). However, in contrast to the easily detected gene editing of HSV after single-meganuclease therapy, gene editing of HSV could not be detected in any of the treated mice by T7E1 assay (FIG. 11A-11C), despite AAV loads equal to or higher than those observed in previous experiments (FIG. 12A). By more-sensitive NGS analysis, only very low levels of mutation (0.1-0.3%) in the Cas9-treated animals could be detected (FIG. 5F).

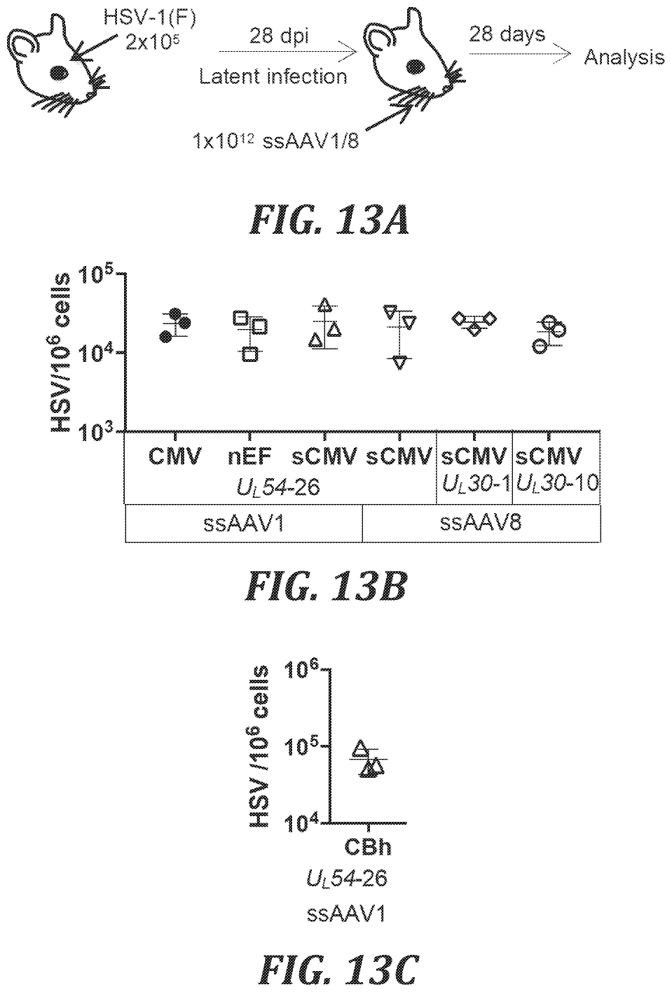

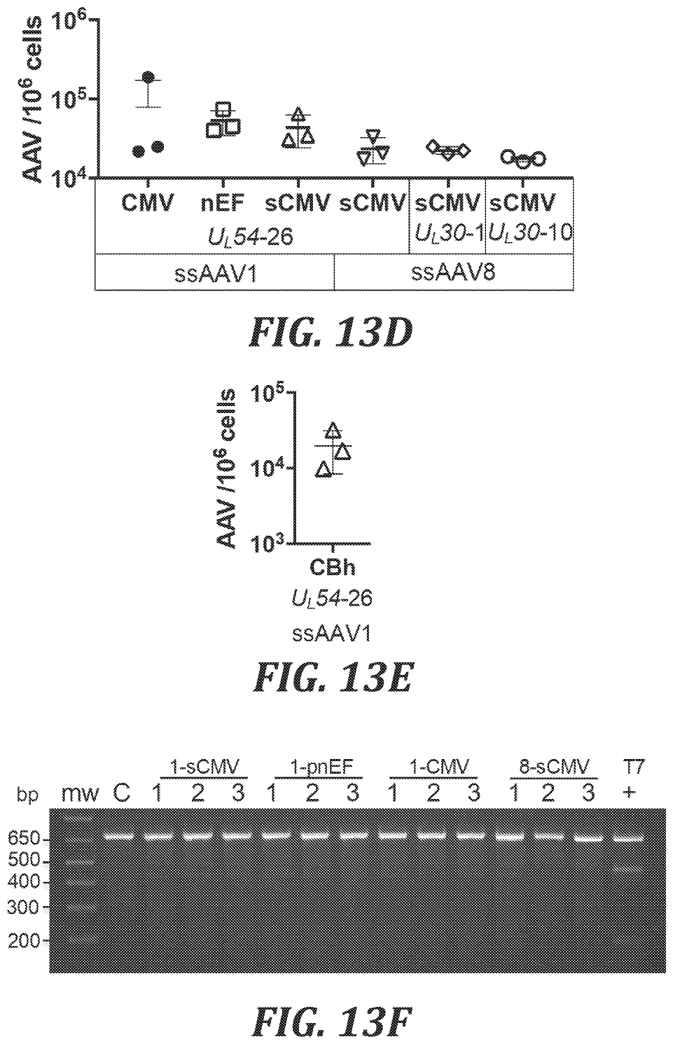

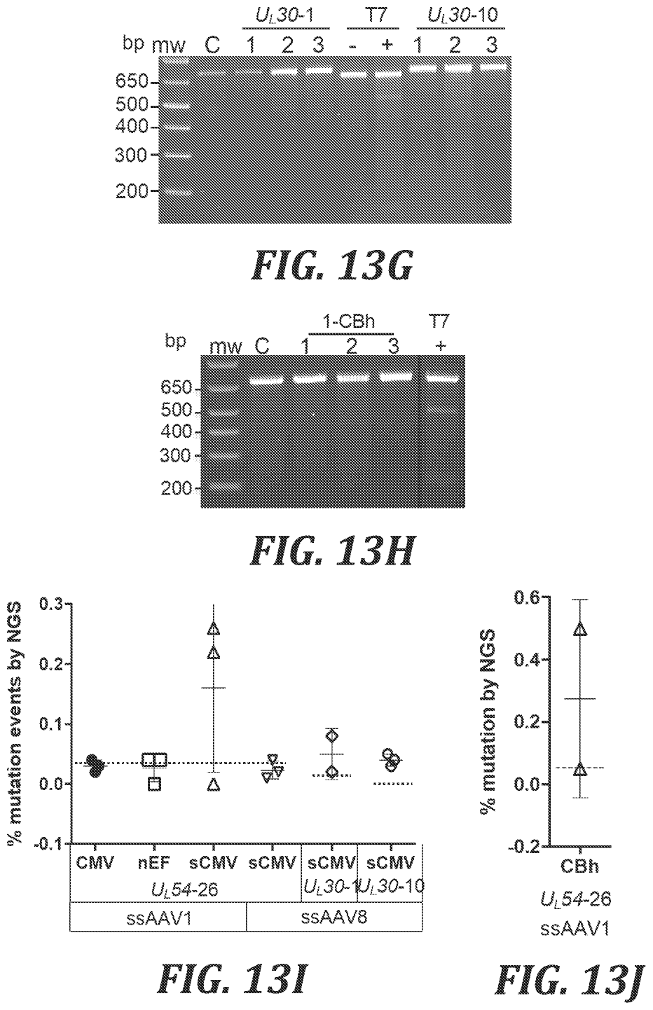

Although the sCMV promoter used in the above experiments with Cas9 can mediate strong transgene expression in sensory neurons in vitro (Aubert, M. et al. In vitro Inactivation of Latent HSV by Targeted Mutagenesis Using an HSV-specific Homing Endonuclease. Mol Ther Nucleic Acids 3, e146, doi: 10.1038/mtna.2013.75 (2014)) and in vivo (Dang, C. H. et al. In vivo dynamics of AAV-mediated gene delivery to sensory neurons of the trigeminal ganglia. Sci Rep 7, 927, doi: 10.1038/s41598-017-01004-y (2017)), the possibility was considered that optimization of the promoter might allow more efficient gene editing of latent HSV by Cas9. To this end, similar in vivo experiments (FIG. 13Aa) were performed using ssAAV1 vectors expressing SaCas9 under control of the alternative strong constitutive promoters CMV, nEF, and CBh. Along with the most effective sgRNA targeting U_{L}54 (sgRNA_{UL54-26}), two additional sgRNAs targeting U_{L}30 (sgRNA_{UL30-1} and sgRNA_{UL30-10}) were also tested. The alternative AAV8 serotype was also assessed, which achieved easily detectable gene editing of HSV when delivering meganucleases (FIGS. 1A-1D). Quantification by ddPCR showed similar levels of HSV in the TG of all treated animals (FIGS. 13B-13C). As observed in the previous experiments, no gene editing of latent HSV genomes was detected by the T7E1 assay under any conditions using Cas9 (FIGS. 13F-13H). In agreement with this and previous results, NGS analysis demonstrated that levels of gene editing were very low, and were observed in only a subset of animals (FIG. 13I-13J).

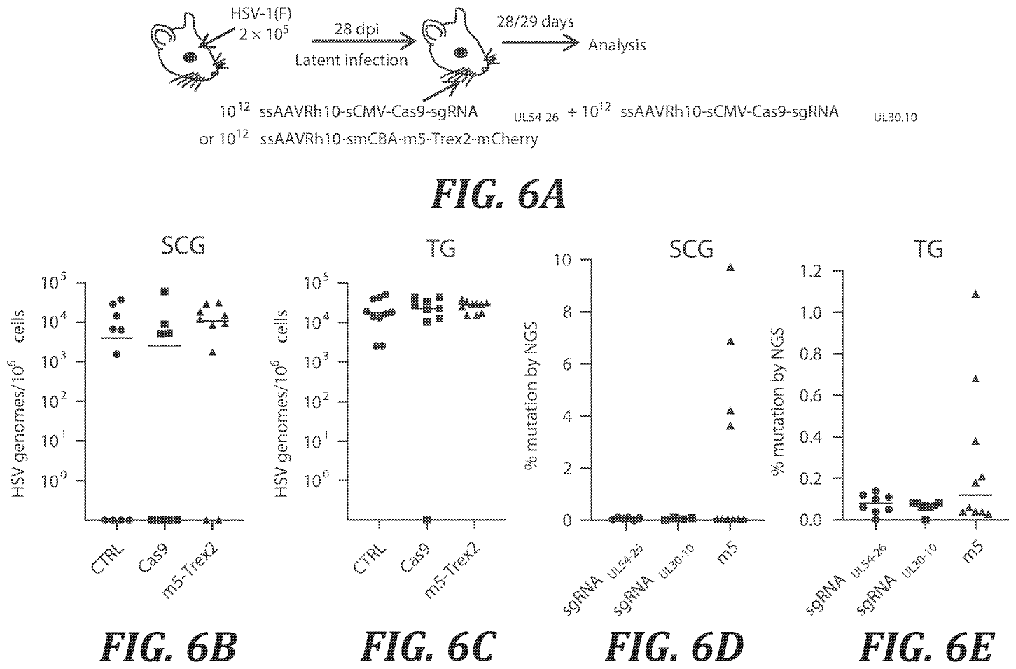

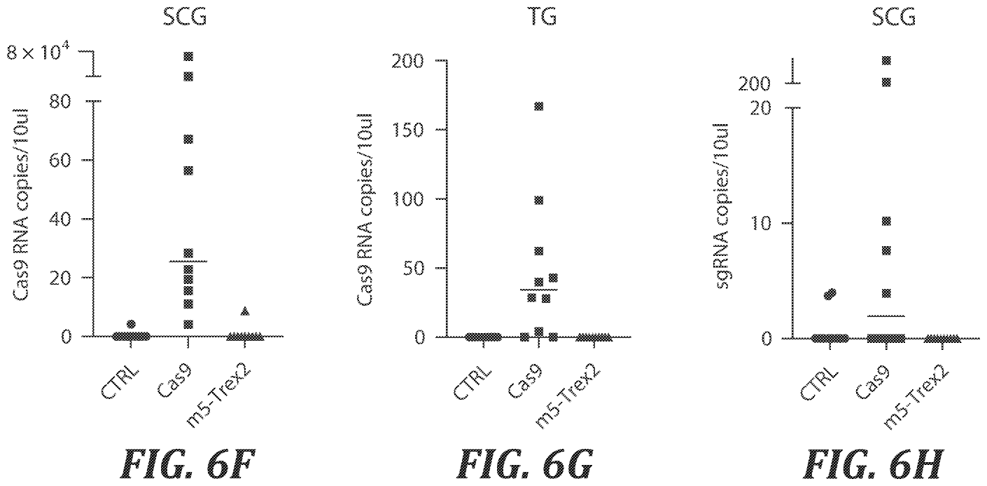

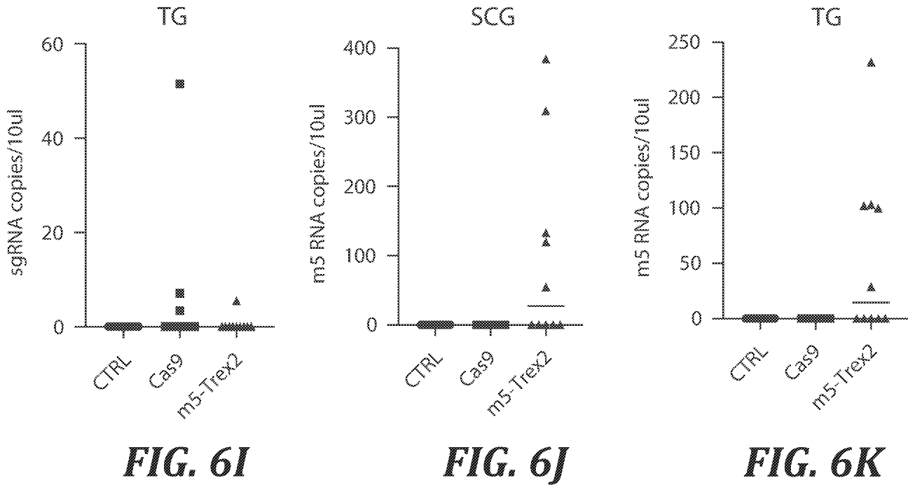

Similar to results using meganucleases, CRISPR/Cas9 has shown significantly higher gene disruption efficiency when targeting dual sites (Wang, G., Zhao, N., Berkhout, B. & Das, A. T. A Combinatorial CRISPR-Cas9 Attack on HIV-1 DNA Extinguishes All Infectious Provirus in Infected T Cell Cultures. Cell Rep 17, 2819-2826, doi:10.1016/j.celrep.2016.11.057 (2016); Lebbink, R. J. et al. A combinational CRISPR/Cas9 gene-editing approach can halt HIV replication and prevent viral escape. Sci Rep 7, 41968, doi:10.1038/srep41968 (2017); van Diemen, F. R. et al. CRISPR/Cas9-Mediated Genome Editing of Herpesviruses Limits Productive and Latent Infections. PLOS Pathog 12, e1005701, doi:10.1371/journal.ppat. 1005701 (2016)). Therefore, it was tested whether dual sgRNA therapy could result in higher gene editing of latent HSV. Latently infected mice were administered either dual sgRNA therapy consisting of 10¹² vg of ssAAVRh10-sCMV-Cas9-sgRNA_{UL54-26} and 10¹² vg of ssAAVRh10-sCMV-Cas9-sgRNA_{UL30-10} or single-meganuclease therapy with 10¹² vg of ssAAVRh10-smCBA-HSV1m5-Trex2-mCherry (FIG. 6A). The ssAAV construct carrying the HSV1m5 also delivers Trex2, a 3′-5′ exonuclease that was shown previously to increase meganuclease gene editing (Aubert, M. et al. In vitro Inactivation of Latent HSV by Targeted Mutagenesis Using an HSV-specific Homing Endonuclease. Mol Ther Nucleic Acids 3, e146, doi:10.1038/mtna.2013.75 (2014)). No loss of HSV genomes was observed in the ganglia of either dual sgRNA/cas9- or single-meganuclease treated mice compared to control animals (FIG. 6B-6C). NGS analysis showed that gene editing was seen in some but not all treated animals regardless of the therapy received, and the levels of mutation observed in ganglia from dual sgRNA treated mice remained weak and lower (<0.2%) than those from ganglia of single meganuclease-treated mice (up to 9.9% in SCG and 1.1% in TG; FIG. 6D-6E). RNA expression of Cas9, sgRNA and m5 was tested to determine whether low enzyme expression could explain the weak gene editing. While Cas9 mRNA was detected in 80% of the SCG and TG from dual sgRNA/cas9 treated mice, only 40% and 20% of mice had detectable levels of sgRNA in SCG and TG, respectively (FIG. 6F-6G). For comparison, with single-meganuclease therapy, expression of HSV1m5 was detected in only 50% of the TG and SCG of treated animals, despite the easily detectable gene editing.

### Single-Neuron Analysis of HSV and AAV Vectors

While optimizing AAV-meganuclease therapy, different levels of HSV gene editing were obtained in ganglia depending on the AAV serotype used for the meganuclease delivery. The route of administration of the AAV delivery vectors influenced the efficiency of HSV gene editing. Delivery of AAV to the sites of HSV latency was shown previously to be less efficient after administration via the cornea (with or without scarification) than intradermal whisker pad (WP) injection (Dang, C. H. et al. In vivo dynamics of AAV-mediated gene delivery to sensory neurons of the trigeminal ganglia. Sci Rep 7, 927, doi:10.1038/s41598-017-01004-y (2017)). The optimization studies presented in FIG. 9 showed that while AAV delivery to ganglia was similar after injection via retroorbital (RO), tail vein (TV) and WP injection, RO led to the highest levels of gene editing, especially with Rh10 serotype. Furthermore, gene editing and loss of viral genomes were consistently greater in SCG than TG (FIGS. 1-4 and 9). These differences were hypothesized to result from dissimilar distribution of AAV vector serotypes and HSV between TG and SCG, and among the different types of neurons within ganglia. To evaluate this issue, AAV-mediated gene delivery to HSV-infected neurons was assessed in mice using single-cell RNA-sequencing (scRNA-seq). Mice were latently infected with HSV-1, after which each latently infected mouse received 10¹² vgs of one of four AAV vector serotypes reported to possess neuronal tropism in mice (Dang, C. H. et al. In vivo dynamics of AAV-mediated gene delivery to sensory neurons of the trigeminal ganglia. Sci Rep 7, 927, doi:10.1038/s41598-017-01004-y (2017); Bradbury, A. M. et al. AAVrh10 Gene Therapy Ameliorates Central and Peripheral Nervous System Disease in Canine Globoid Cell Leukodystrophy (Krabbe Disease). Hum Gene Ther 29, 785-801, doi:10.1089/hum.2017.151 (2018); Chan, K. Y. et al. Engineered AAVs for efficient noninvasive gene delivery to the central and peripheral nervous systems. Nat Neurosci 20, 1172-1179, doi:10.1038/nn.4593 (2017)). Each AAV serotype carried a unique marker transgene: AAV1-mScarlet; AAV8-mEGFP; AAV-PHP.S-DsRed-Express2; and AAV-Rh10-mTagBFP-2. Three weeks after AAV injection, TG and SCG were collected and TG or SCG pooled from all animals for neuron purification, library construction, and sequencing. High quality single cell expression data was obtained from 2,319 purified TG neurons and 2,041 SCG neurons (99,817 mean reads and 5,908 median genes per cell for TG; 94,797 mean reads and 5,635 median genes per cell for SCG).

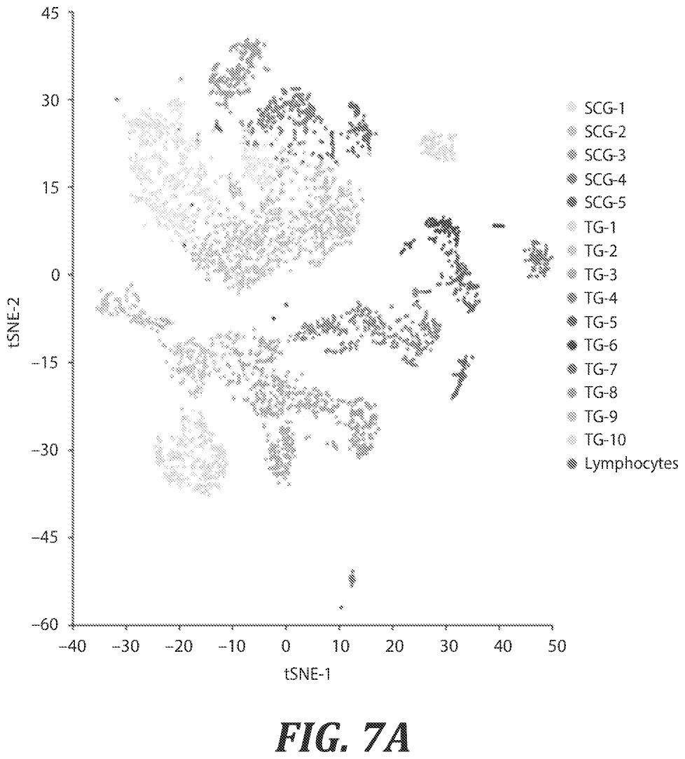

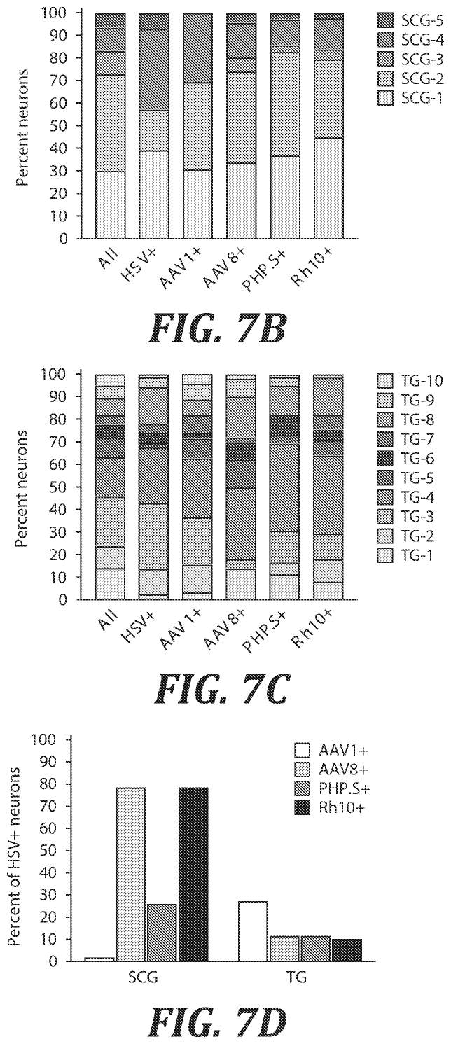

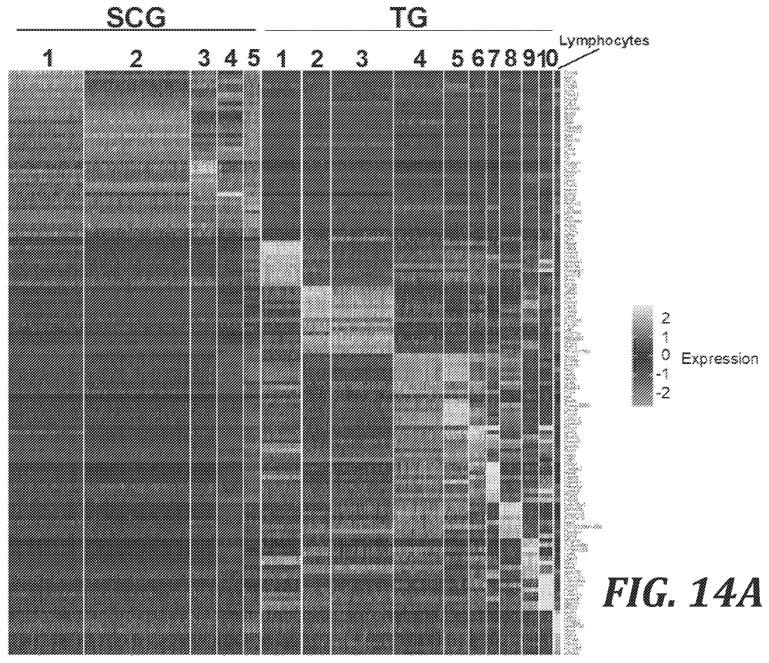

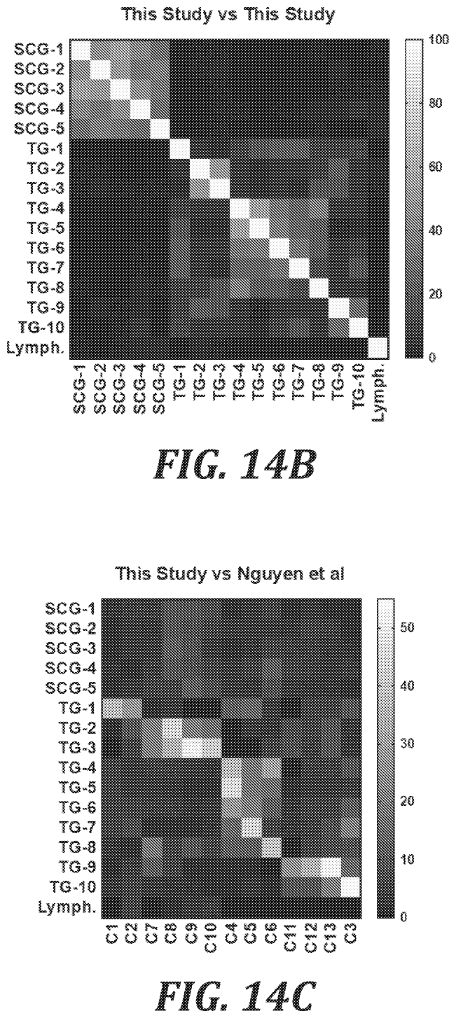

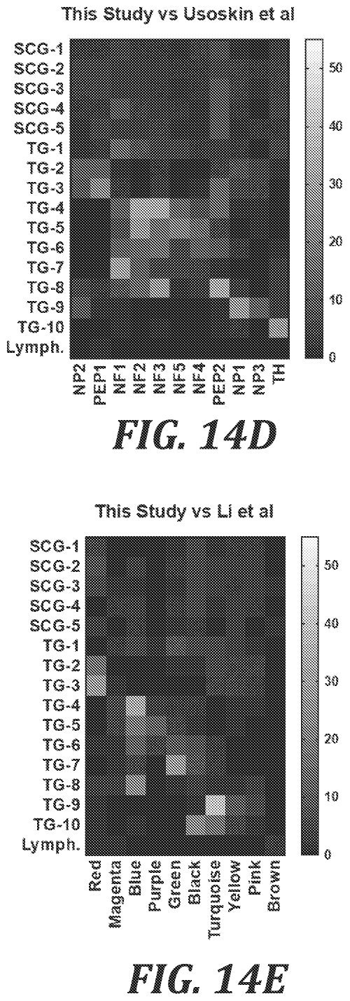

To determine whether specific subtypes of neurons were infected by HSV and targeted by each AAV serotype, neurons were first classified into groups based on gene expression profiles. Principal Component Analysis (PCA) of the single cell gene expression data identified transcriptionally distinct clusters of neurons for each tissue: 5 for the SCG and 10 for the TG. As expected, SCG (autonomic) and TG (sensory) neurons fully segregated from one another (FIGS. 7A and 14A). The cell clusters in the TG correlated well with clusters identified previously in the DRG (Usoskin, D. et al. Unbiased classification of sensory neuron types by large-scale single-cell RNA sequencing. Nat Neurosci 18, 145-153, doi:10.1038/nn.3881 (2015); Li, C. L. et al. Somatosensory neuron types identified by high-coverage single-cell RNA-sequencing and functional heterogeneity. Cell Res 26, 83-102, doi:10.1038/cr.2015.149 (2016)) and the TG (Nguyen, M. Q., Wu, Y., Bonilla, L. S., von Buchholtz, L. J. & Ryba, N. J. P. Diversity amongst trigeminal neurons revealed by high throughput single cell sequencing. PLOS One 12, e0185543, doi:10.1371/journal.pone.0185543 (2017)). For example, cluster TG-8 corresponds closely to Cluster 6 in Nguyen (Nguyen et al. PLOS One 12, e0185543, doi:10.1371/journal.pone.0185543 (2017)), cluster PEP2 in Usoskin (Usoskin et al. Nat Neurosci 18, 145-153, doi:10.1038/nn.3881 (2015)), and cluster C8-2 in Li (Li et al. Cell Res 26, 83-102, doi:10.1038/cr.2015.149 (2016)) (FIGS. 14B-14E).

In order to determine the relative distribution of HSV and each AAV serotype across neuronal subtypes, neurons were identified that expressed HSV transcripts, as well as each of the marker transgenes carried by the four different AAV serotypes (mScarlet (AAV1), mEGFP (AAV8), DsRed-Express2 (AAV-PHP.S), and mTagBFP-2 (AAV-Rh10)) (FIG. 7 and Tables 3-4).

TABLE 3
Total cell count in each cluster (Normalized to input)
a. TG clusters (Normalized to input)

| | TG-1 | TG-2 | TG-3 | TG-4 | TG-5 | TG-6 | TG-7 | TG-8 | TG-9 | TG-10 | Total TG |
|---|---|---|---|---|---|---|---|---|---|---|---|
| HSV+ | 7 | 32 | 82 | 69 | 6 | 13 | 11 | 46 | 12 | 4 | 282 |
| AAV1+ | 15 | 51 | 88 | 110 | 37 | 11 | 33 | 29 | 29 | 18 | 421 |
| AAV8+ | 39 | 0 | 11 | 88 | 33 | 22 | 6 | 50 | 22 | 6 | 277 |
| AAV− | 33 | 15 | 40 | 110 | 11 | 26 | 0 | 37 | 11 | 4 | 287 |
| PHP.S+ | | | | | | | | | | | |
| AAV− | 18 | 22 | 26 | 77 | 15 | 11 | 15 | 37 | 0 | 4 | 225 |
| Rh10+ | | | | | | | | | | | |
| neurons | 328 | 230 | 505 | 406 | 197 | 136 | 97 | 177 | 130 | 113 | 2319 |

b. SCG clusters (Normalized to input)

| | SCG-1 | SCG-2 | SCG-3 | SCG-4 | SCG-5 | Total SCG |
|---|---|---|---|---|---|---|
| HSV+ | 11 | 5 | 0 | 10 | 2 | 28 |
| AAV1+ | 15 | 18 | 0 | 15 | 0 | 48 |
| AAV8+ | 418 | 495 | 77 | 187 | 55 | 1232 |
| AAV−PHP.S+ | 143 | 176 | 11 | 44 | 11 | 385 |
| AAV−Rh10+ | 216 | 165 | 22 | 66 | 11 | 480 |
| Neurons | 616 | 871 | 211 | 208 | 135 | 2041 |

TABLE 4
Percent neurons HSV+ or AAV+ in each cluster (Normalized to input)
a. TG clusters (Normalized in input)

| | TG-1 | TG-2 | TG-3 | TG-4 | TG-5 | TG-6 | TG-7 | TG-8 | TG-9 | TG-10 | Total TG |
|---|---|---|---|---|---|---|---|---|---|---|---|
| HSV+ | 2.1 | 13.9 | 16.2 | 17.0 | 3.0 | 9.6 | 11.3 | 26.0 | 9.2 | 3.5 | 12.2 |
| AAV1+ | 4.5 | 22.3 | 17.4 | 27.1 | 18.6 | 8.1 | 34.0 | 16.6 | 22.6 | 16.2 | 18.2 |
| AAV8+ | 11.7 | 0.0 | 2.2 | 21.7 | 16.8 | 16.2 | 5.7 | 28.0 | 16.9 | 4.9 | 11.9 |
| AAV− | 10.1 | 6.4 | 8.0 | 27.1 | 5.6 | 18.9 | 0.0 | 20.7 | 8.5 | 3.2 | 12.3 |
| PHP.S+ | | | | | | | | | | | |
| AAV− | 5.6 | 9.6 | 5.1 | 19.0 | 7.4 | 8.1 | 15.1 | 20.7 | 0.0 | 3.2 | 9.6 |
| Rh10+ | | | | | | | | | | | |

b. SCG clusters (Normalized to input)

| | SCG-1 | SCG-2 | SCG-3 | SCG-4 | SCG-5 | Total SCG |
|---|---|---|---|---|---|---|
| HSV+ | 1.8 | 0.6 | 0.0 | 4.8 | 1.5 | 1.4 |
| AAV1+ | 2.4 | 2.1 | 0.0 | 7.1 | 0.0 | 2.3 |
| AAV8+ | 67.9 | 56.8 | 36.5 | 89.9 | 40.7 | 60.4 |
| AAV−PHP.S+ | 23.2 | 20.2 | 5.2 | 21.2 | 8.1 | 18.9 |
| AAV−Rh10+ | 35.1 | 18.9 | 10.4 | 31.7 | 8.1 | 23.5 |

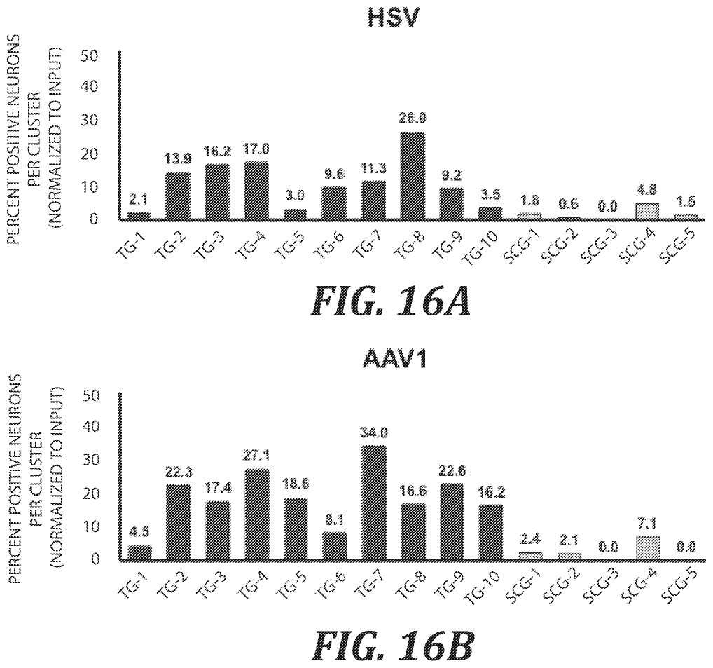

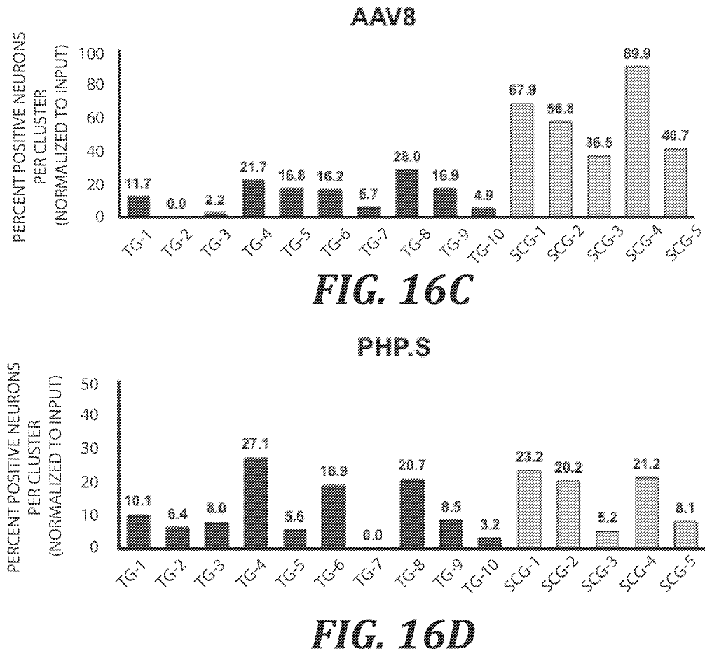

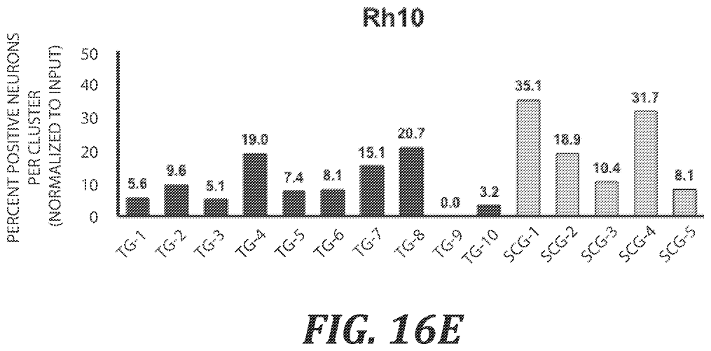

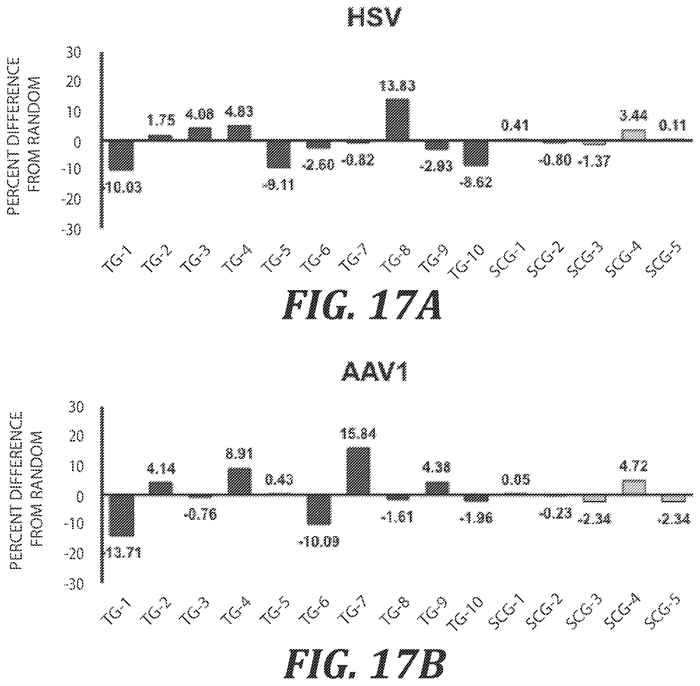

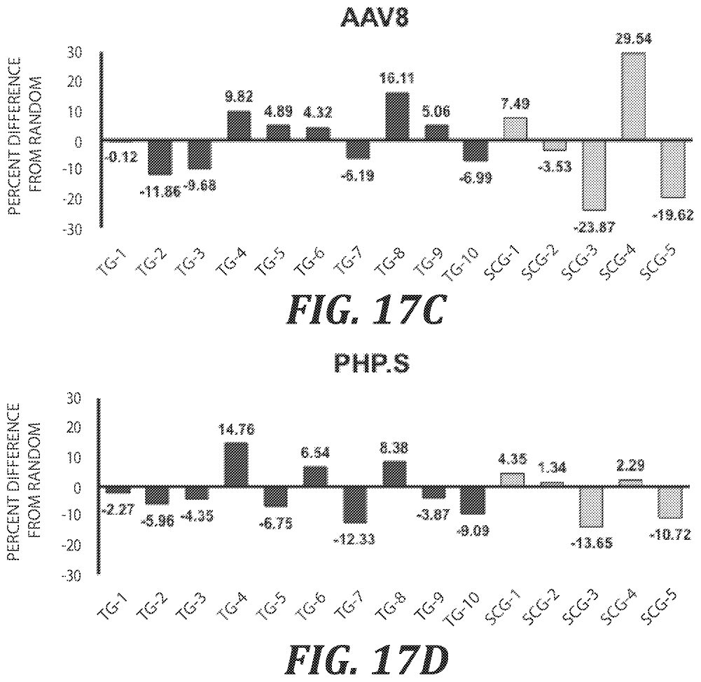

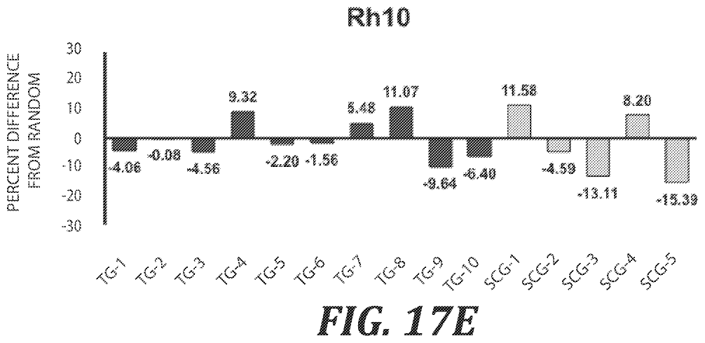

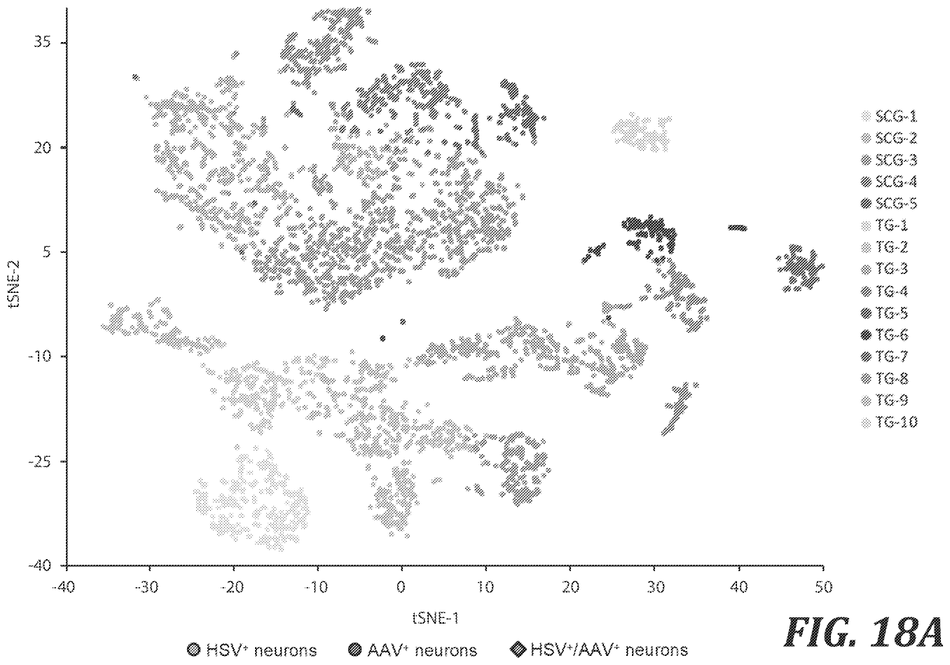

The latency-associated transcript (LAT), the only HSV RNA highly expressed during latent infection (reviewed in Fields, B., Knipe, D., Howley, P. & Griffin, D. (Philadelphia: Wolters Kluwer Health/Lippincott Williams & Wilkins, 2007)), accounted for >99% of all HSV transcripts detected. HSV transcripts were detected in 1.4% of cells in the SCG and 12.2% of cells in the TG, consistent with ddPCR results that showed 10-fold fewer HSV genomes in the SCG (FIG. 9). HSV-expressing cells were non-randomly distributed across the different neuronal clusters within both the SCG (χ², p<0.0001) and the TG (χ², p<0.0001). For example, in the SCG, HSV-expressing cells were most enriched in SCG-4, and absent from SCG-3, whereas in the TG HSV-expressing cells were most enriched in TG-8 (FIGS. 7B-7C, 16, and 17 and Tables 3-4).

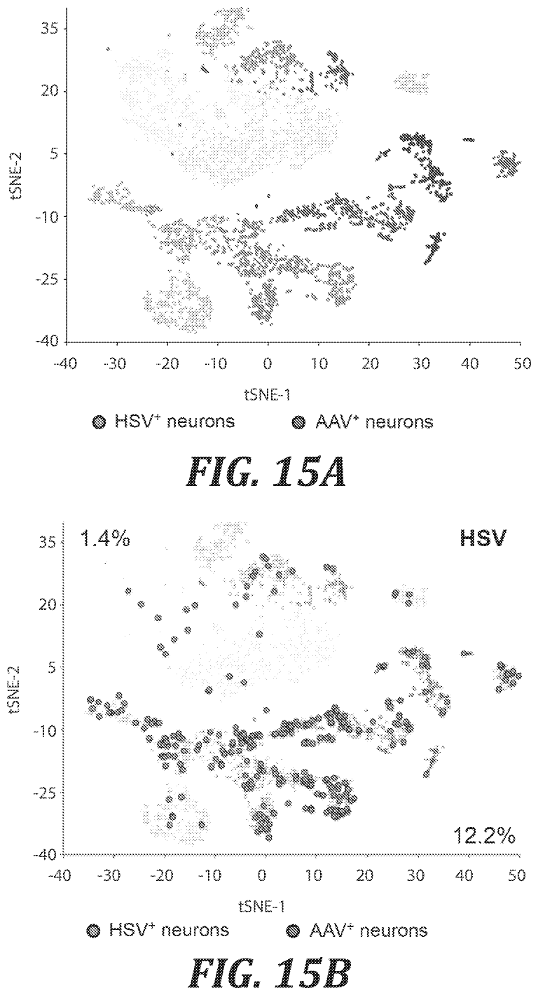

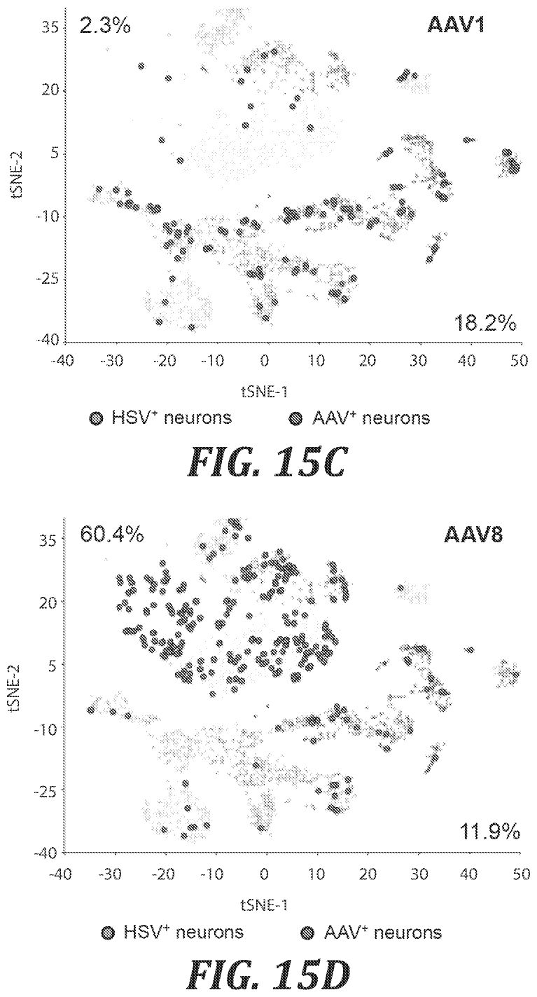

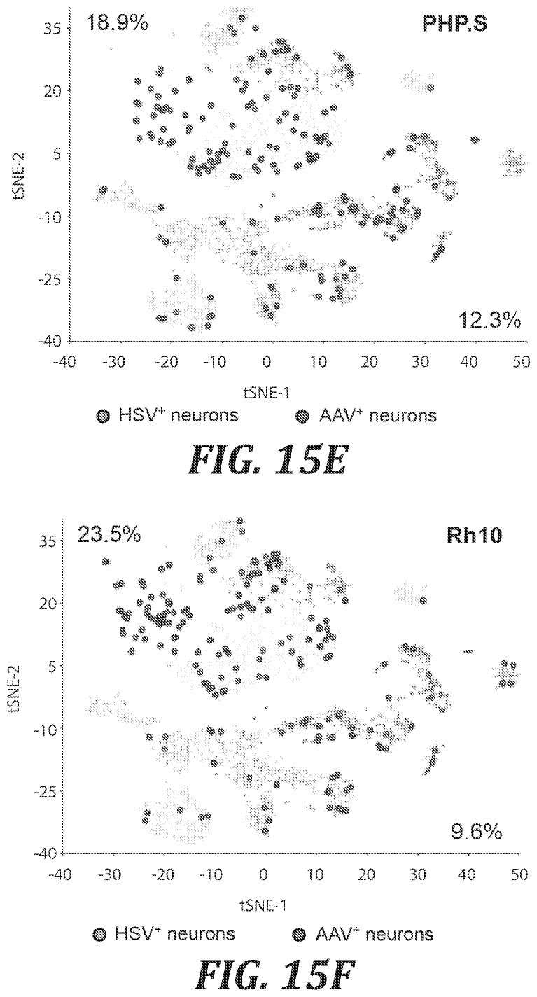

The distribution of transgene expression from AAV varied by serotype and tissue. AAV1 (mScarlet) supported minimal expression in the SCG, with only 2.3% of neurons expressing mScarlet, but supported the broadest transgene expression in the TG (18.2% of cells). By contrast, mEGFP (AAV8) was expressed in 60.4% of SCG neurons and 11.9% of TG neurons, DsRed-Express2 (PHP.S) was expressed in 18.9% and 12.3% of neurons in the SCG and TG respectively, and mTagBFP-2 (Rh10) was expressed in 23.5% and 9.6% of SCG and TG neurons respectively (FIG. 15). Within the SCG, mScarlet (AAV1)-expressing cells were randomly distributed across the clusters, while mEGFP (AAV8), DsRed-Express2 (AAV-PHP.S) and mTagBFP-2 (AAV-Rh10) exhibited a non-random distribution (χ²: p<0.0981, p<0.0001, p=0.0143, and p<0.0001, respectively; FIGS. 7B, 16, and 17 and Tables 2-3). Within the TG, transgene expression from all serotypes was non-randomly distributed across the neuronal clusters (χ²: AAV1 p=0.0023; AAV8 p=0.0002; PHP.S p<0.0001; Rh10 p=0.0006; FIGS. 7C, 16, and 17 and Tables 3-4). 62% of AAV-Rh10+ neurons vs. 54% of AAV8+ neurons were collectively in TG-3, TG-4 and TG-8, the neuronal clusters which contained 70% of the HSV⁺ neurons, which may partially explain why Rh10 provided slightly higher levels of gene editing in the TG than did AAV8 (FIG. 9). The analysis did not identify specific cellular transcripts unique to neurons transduced by specific AAV serotypes.

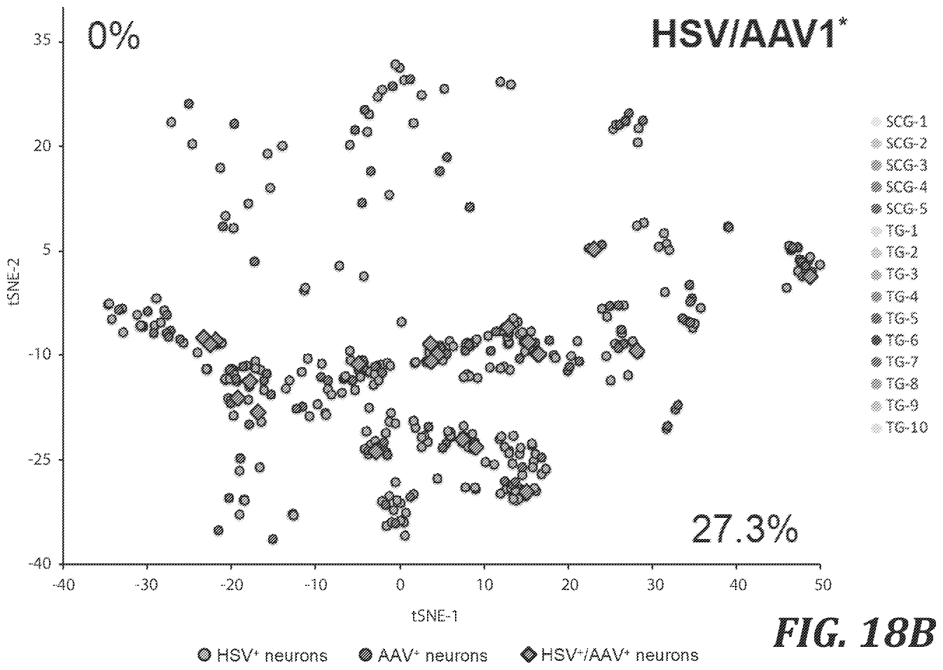

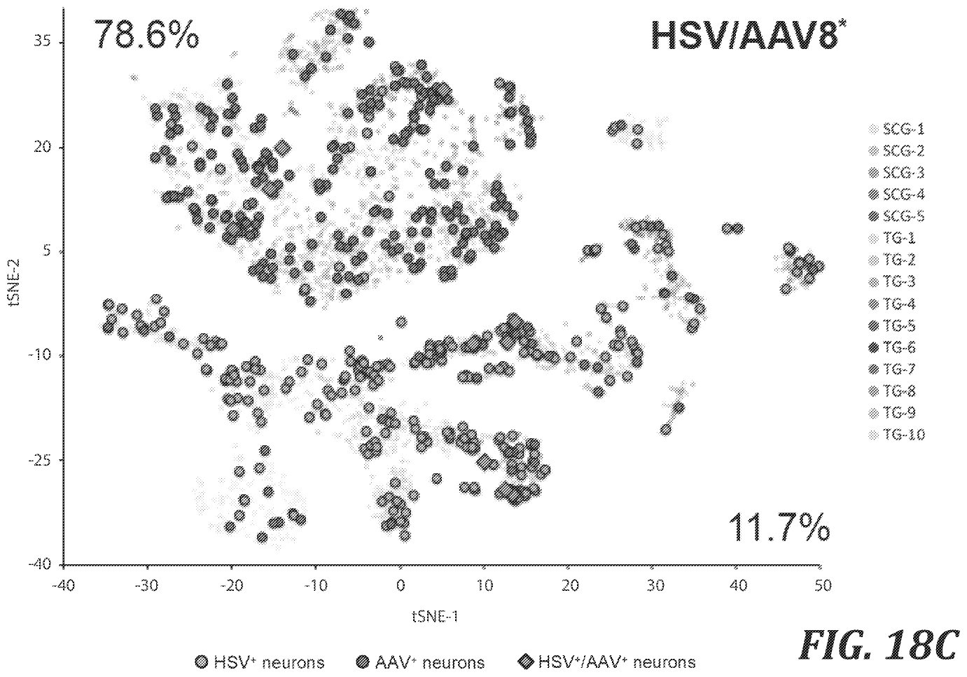

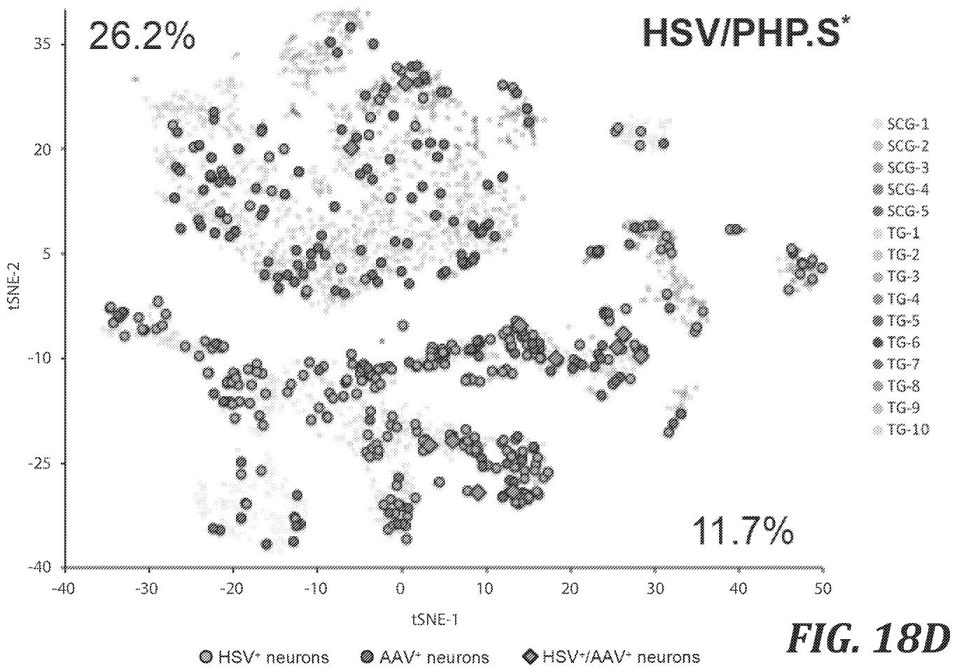

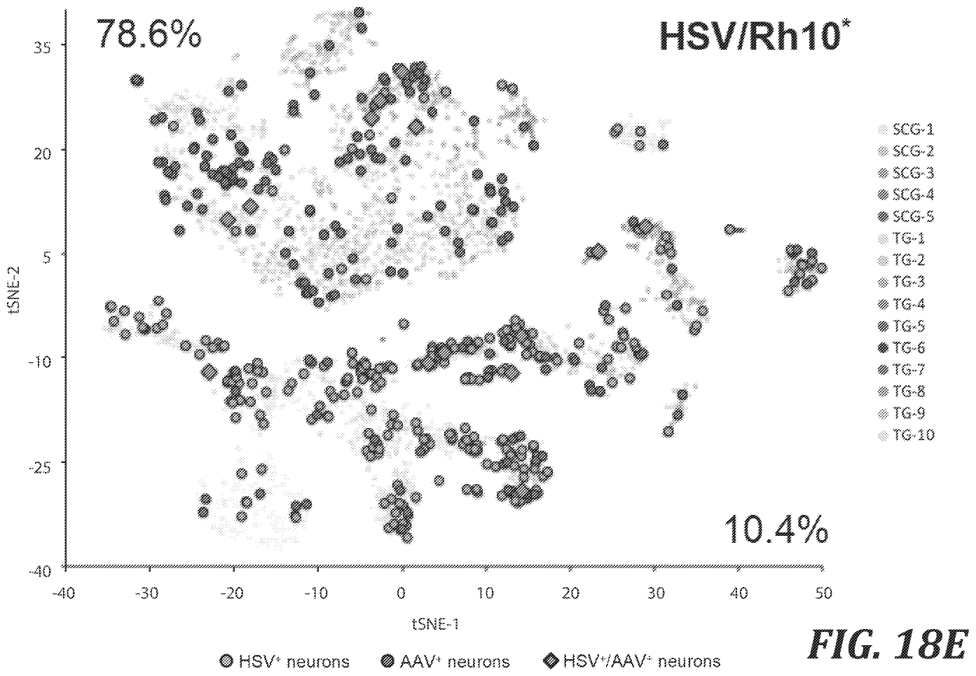

Finally, the overlap of HSV gene expression compared with each AAV serotype across neuronal clusters was evaluated. Strikingly, 79% of HSV⁺ neurons in the SCG were calculated to also express an AAV8 or Rh10 transgene, whereas only approximately 10% of HSV⁺ cells in the TG detectably expressed those transgenes (FIGS. 7D, 18 and Table 5).

TABLE 5
Percent HSV+ AAV+ neurons in each cluster (Normalized to input)
a. TG clusters (Normalized to input)

| | TG-1 | TG-2 | TG-3 | TG-4 | TG-5 | TG-6 | TG-7 | TG-8 | TG-9 | TG-10 | Total TG |
|---|---|---|---|---|---|---|---|---|---|---|---|
| AAV1+ | 0.0 | 34.4 | 26.8 | 42.5 | 0.0 | 28.2 | 33.3 | 15.9 | 0.0 | 0.0 | 27.3 |
| AAV8+ | 0.0 | 0.0 | 0.0 | 23.9 | 0.0 | 0.0 | 0.0 | 35.9 | 0.0 | 0.0 | 11.7 |
| AAV− | 0.0 | 0.0 | 8.9 | 26.6 | 0.0 | 0.0 | 0.0 | 15.9 | 0.0 | 0.0 | 11.7 |
| PHP.S+ | | | | | | | | | | | |
| AAV− | 0.0 | 11.5 | 0.0 | 21.3 | 0.0 | 56.4 | 0.0 | 8.0 | 0.0 | 0.0 | 10.4 |
| Rh10+ | | | | | | | | | | | |

b. SCG clusters (Normalized to input)

| | SCG-1 | SCG-2 | SCG-3 | SCG-4 | SCG-5 | Total SCG |
|---|---|---|---|---|---|---|
| AAV1+ | 0.0 | 0.0 | 0.0 | 0.0 | 0.0 | 0.0 |
| AAV8+ | 100.0 | 0.0 | 0.0 | 55.0 | 0.0 | 78.6 |
| AAV−PHP.S+ | 33.3 | 0.0 | 0.0 | 36.7 | 0.0 | 26.2 |
| AAV−Rh10+ | 66.7 | 0.0 | 0.0 | 100.0 | 0.0 | 78.6 |

These scRNA-seq data collectively demonstrate that the distribution of AAV to HSV-containing neurons is a crucial parameter in modulating DNA editing efficiency, and suggest that choosing AAV serotypes, promoters, and delivery methods to maximize the overlap of AAV transduction with HSV infection might increase the efficacy of meganuclease gene therapy against HSV.

### Combination of AAV Serotypes Leads to Higher Gene Editing

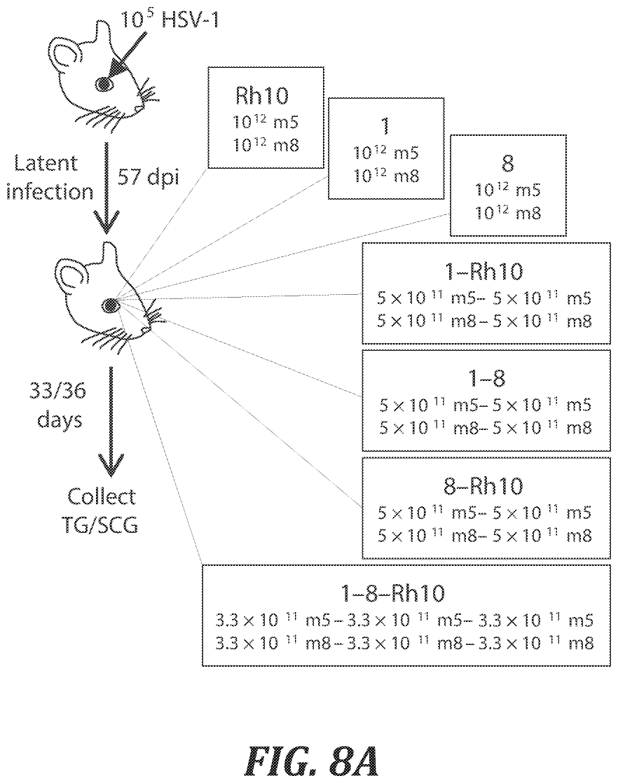

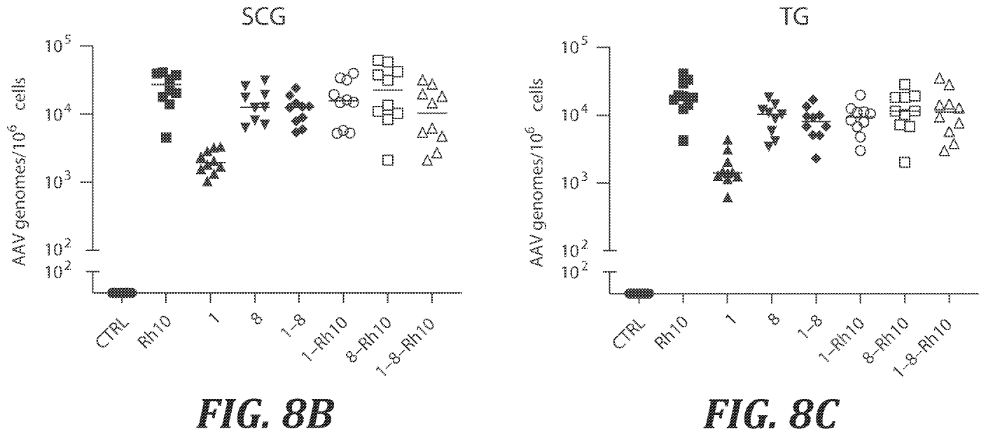

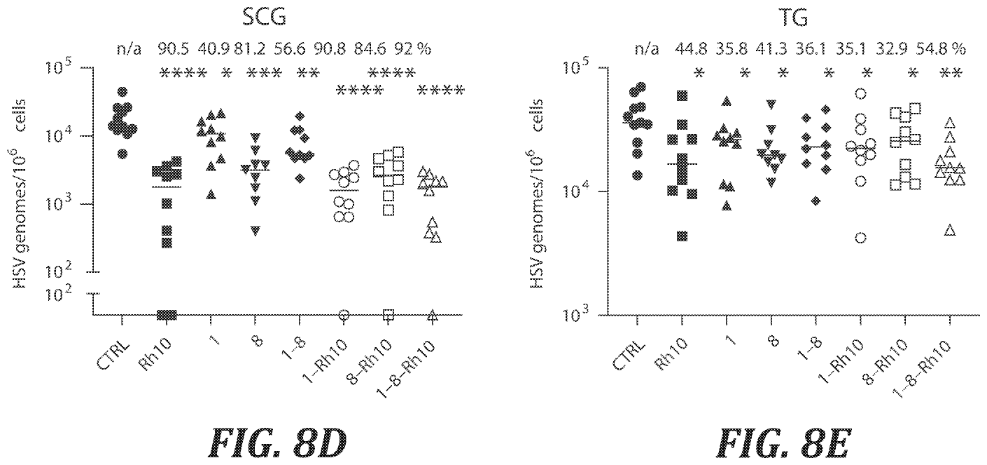

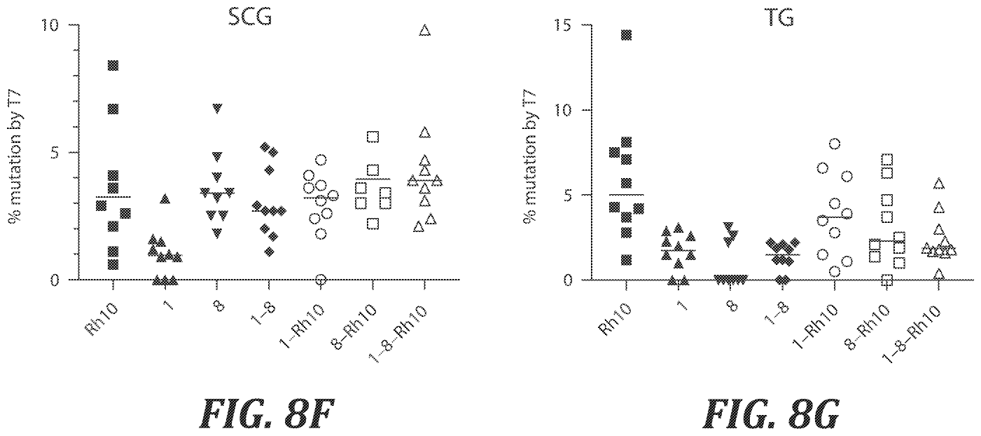

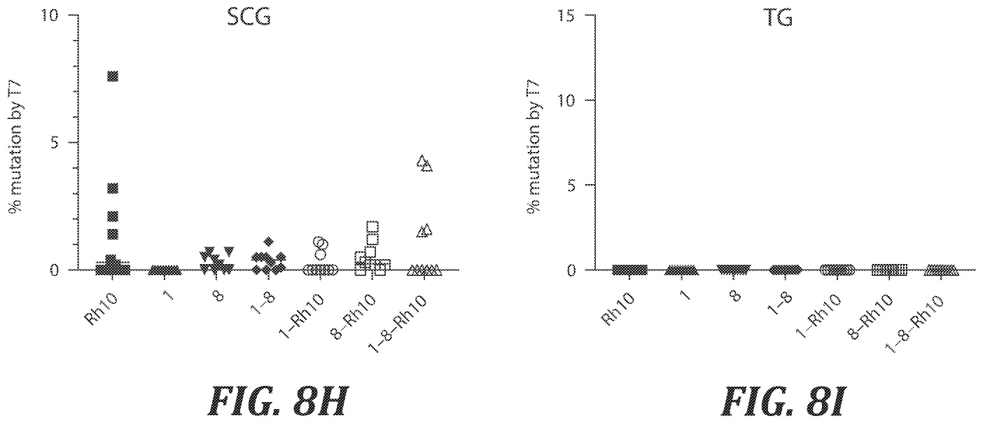

The results from the scRNA-seq analysis indicated that individual AAV serotypes vary in their tropism for specific neuronal subsets, suggesting that a combination of AAV serotypes could facilitate efficient ganglionic meganuclease delivery. Therefore, latently infected mice were treated with dual-meganuclease therapy using either single (AAV1, AAV8 or AAVRh10), double (AAV1 and AAV8, AAV1 and AAVRh10, or AAV8 and AAVRh10) or triple (AAV1, AAV8 and AAVRh10) AAV serotype combinations (FIG. 8A). Analysis of SCG and TG collected a month later showed that all AAV combinations, except for AAV1 alone, transduced the ganglia at similar levels (FIG. 8B-8C). Consistent with the results above, dual-meganuclease therapy delivered using AAVRh10, either alone or in combination with 1 or 2 of the other AAV serotypes, led to the highest loss of latent viral genomes from SCG, with the greatest decrease in viral load obtained in the triple AAV combination (92%, FIG. 8B, Table 6).

TABLE 6
HSV loads after delivery of dual meganuclease
therapy using AAV combinations

| | | Percent | |
|---|---|---|---|
| Groups | Mean viral load per | reduction | P values |
| (n = 9-11 mice) | 1 × 10⁶ SCG/TG | SCG/TG % | SCG/TG |
| CTRL | 1.87 × 10⁴/2.17 × 10⁴ | n/a | n/a |
| Rh10 | 1.78 × 10³/2.52 × 10⁴ | 90.5/44.8 | <10⁻⁴/0.013 |
| 1 | 1.10 × 10⁴/2.30 × 10⁴ | 40.9/35.8 | 0.034/0.026 |
| 8 | 3.51 × 10³/2.51 × 10⁴ | 81.2/41.3 | 0.0003//0.013 |
| 1-8 | 8.14 × 10³/2.55 × 10⁴ | 56.6/36.1 | 0.005/0.02 |
| 1-Rh10 | 1.73 × 10³/2.64 × 10⁴ | 90.8/35.1 | <10⁻⁴/0.036 |
| 8-Rh10 | 2.89 10³/2.17 10⁴ | 84.6/32.9 | <10⁻⁴/0.036 |
| 1-8-Rh10 | 1.49 × 10³/1.78 × 10⁴ | 92.0/54.8 | <10⁻⁴/0.001 |

CTRL: Control;
n/a: not applicable
All data are presented as mean values and statistical analysis was conducted using one-tail, unpaired t-test.
Source data are provided as a Source Data file.

As predicted from our scRNA-seq data, the smallest decrease in HSV genomes was observed in animals receiving AAV1, unless AAVRh10 was included. Similarly, in the TG, the greatest loss of latent HSV genomes was detected in mice having received the dual-meganuclease therapy delivered using the triple AAV serotype combination (54.8%, FIG. 8E, Table 6). In agreement with previous results, despite the efficient elimination of HSV genomes, gene editing in the residual HSV genomes was low, generally <10% for the HSV1m5 site (FIG. 8F-8G) and ranging from undectable to 8% for the HSVm8 site (FIG. 8H-8I).

Taken together, these data suggest that in order to maximize HSV gene therapy in both SCG and TG, meganuclease delivery may benefit from a combination of different AAV serotypes to optimally target all HSV-infected neurons in both autonomic and sensory ganglia. Furthermore, the results showed that AAV1 may not be an ideal serotype to be used in combination with AAVRh10 and AAV8. Additional experimentation will be required to identify the optimal AAV serotype(s) to include in therapeutic combinations to maximize the meganuclease delivery to all HSV-infected ganglionic neurons.

### DISCUSSION

This Examples describes the use of a relatively simple mouse model of HSV infection to perform a set of iterative studies to increase the efficiency of AAV-delivered gene editing enzymes targeting HSV. This Examples discloses a reduction in HSV genomes of >90% in SCG and >50% in TG of treated animals. This represents a dramatic improvement upon the inventors' previous report in which a maximum of about 4% gene editing with no loss of viral genomes was observed. While HSV does not reactivate spontaneously from ganglia of living mice, the virus does reactivate from mouse neurons after explantation, and the results demonstrate 95% (SCG) to 55% (TG) reduction in viral genomes produced de novo in ganglionic explants after meganuclease treatment of latently infected mice. This is consistent with previous work demonstrating that ganglionic HSV load is a major determinant of the frequency of viral reactivation (Hoshino, Y., Pesnicak, L., Cohen, J. I. & Straus, S. E. Rates of reactivation of latent herpes simplex virus from mouse trigeminal ganglia ex vivo correlate directly with viral load and inversely with number of infiltrating CD8+ T cells. J Virol 81, 8157-8164, doi:10.1128/JVI.00474-07 (2007); Sawtell, N. M. The probability of in vivo reactivation of herpes simplex virus type 1 increases with the number of latently infected neurons in the ganglia. J Virol 72, 6888-6892 (1998); Sawtell, N. M., Poon, D. K., Tansky, C. S. & Thompson, R. L. The latent herpes simplex virus type 1 genome copy number in individual neurons is virus strain specific and correlates with reactivation. J Virol 72, 5343-5350 (1998); Hoshino, Y., Pesnicak, L., Straus, S. E. & Cohen, J. I. Impairment in reactivation of a latency associated transcript (LAT)-deficient HSV-2 is not solely dependent on the latent viral load or the number of CD8(+) T cells infiltrating the ganglia. Virology 387, 193-199, doi:10.1016/j.virol.2009.02.004 (2009)). If translated to humans, such an outcome could be useful in reducing the likelihood of viral reactivation, shedding, and transmission to others (Schiffer, J. T., Mayer, B. T., Fong, Y., Swan, D. A. & Wald, A. Herpes simplex virus-2 transmission probability estimates based on quantity of viral shedding. J R Soc Interface 11, 20140160, doi:10.1098/rsif.2014.0160 (2014)). The inventors had previously hypothesized that linearization of episomal latent HSV genomes by gene editing enzymes might lead to their degradation and loss if DNA repair and recircularization were unsuccessful (Aubert, M. et al. In vivo disruption of latent HSV by designer endonuclease therapy. JCI Insight 1, doi:10.1172/jci.insight.88468 (2016)). However, the gene editing frequencies achieved in the inventors' earlier work following exposure to a single meganuclease were insufficient to generate enough HSV genome loss to be detected, even by precise ddPCR assays (Aubert, M. et al. In vivo disruption of latent HSV by designer endonuclease therapy. JCI Insight 1, doi:10.1172/jci.insight.88468 (2016)). In contrast, the robust gene editing achieved in the current report after dual-meganuclease therapy led to loss of HSV genomes to a degree readily detectable by ddPCR. Of the remaining viral genomes, an average of 4% to 6% were mutated, which likely further contributes to suppressing HSV reactivation.

One limitation to this investigation is that one cannot distinguish whether the observed reduction in de novo production of viral genomes resulted from a reduction in the number of reactivation events, or instead from a decrease in virus production after a reactivation event has initiated. The inventors' previous results suggest that newly synthesized HSV genomes are efficiently targeted by nucleases (Aubert, M. et al. In vivo disruption of latent HSV by designer endonuclease therapy. JCI Insight 1, doi: 10.1172/jci.insight.88468 (2016)), while results from various groups demonstrate that chromatin modification of latent HSV can reduce the efficacy of gene editing (Aubert, M. et al. In vivo disruption of latent HSV by designer endonuclease therapy. JCI Insight 1, doi:10.1172/jci.insight.88468 (2016); Aubert, M. et al. In vitro Inactivation of Latent HSV by Targeted Mutagenesis Using an HSV-specific Homing Endonuclease. Mol Ther Nucleic Acids 3, e146, doi: 10.1038/mtna.2013.75 (2014); van Diemen, F. R. et al. CRISPR/Cas9-Mediated Genome Editing of Herpesviruses Limits Productive and Latent Infections. PLOS Pathog 12, e1005701, doi:10.1371/journal.ppat. 1005701 (2016); Oh, H. S. et al. Herpesviral lytic gene functions render the viral genome susceptible to novel editing by CRISPR/Cas9. Elife 8, doi:10.7554/eLife.51662 (2019)). This has important implications for the ultimate embodiment of gene editing for HSV, as it will determine whether sustained long-term expression of nuclease (as achieved using AAV vectors (Dang, C. H. et al. In vivo dynamics of AAV-mediated gene delivery to sensory neurons of the trigeminal ganglia. Sci Rep 7, 927, doi:10.1038/s41598-017-01004-y (2017)) is required for therapeutic benefit, or whether alternative transient delivery approaches might be sufficient. On the other hand, the substantial improvement in reduction of latent HSV load reported here (>90% reduction compared to approximately 4% in the inventors' previous report) warrant cautious optimism that with further optimization, the ganglionic HSV load might be reduced to levels at which sustained nuclease expression is unnecessary. Ongoing experiments in mice and other model systems will shed additional light on this issue.

The disclosure in this Example convincingly demonstrates the value of single-cell analysis for optimizing in vivo gene therapy. For gene editing against persistent viruses to have maximum efficacy, transgene must be efficiently delivered to cells containing virus. These results demonstrate that a substantial fraction of HSV-expressing cells also express detectable reporter transgene indicating delivery by AAV, particularly in the SCG where editing was highest. Furthermore, these results clearly demonstrate that distinct neuronal subsets are preferentially transduced by different AAV serotypes. For example, AAV1 transduced 17.4% of cells in TG-3, 16.6% of cells in TG-8, but only 2.3% of cells across the whole SCG, none of which were HSV⁺, while AAVRh10 transduced only 5.1% of cells in TG-3, but 20.7% of cells in TG-8 and 23.5% of cells across the SCG including 78.6% of the HSV expressing cells. Together these results suggested that combinations of AAV serotypes may be required to efficiently target all neurons containing latent HSV, which were tested in a follow-up experiment where rationally-selected AAV serotype combinations were used for the delivery of dual meganuclease therapy. As predicted, the triple AAV serotype combination led to the greatest decrease in HSV loads, 92% for the SCG and 54.8% for the TG. These results suggest that the AAV serotypes combined for the delivery of meganuclease therapy need to be carefully chosen, and likely can be further improved. For example, the use of AAV1 alone resulted in the lowest decrease in HSV loads in both SCG and TG, and the addition of AAV1 appeared to add little efficacy to AAVRh10 or AAV8, either alone or in combination. Future work should focus on identifying the optimal combinations of AAV to ensure full coverage of all infected neurons, and to establish whether such combinations are also effective in other model systems, which would facilitate human translation of this approach.

Somewhat surprisingly, in the disclosed system meganucleases provided substantially higher gene editing than did Cas9 with any of the tested gRNAs. The simplest explanation for this may be due to relative expression levels; due to its larger size Cas9 requires delivery by single-stranded (ss)AAV vectors, while meganucleases fit easily into the more transcriptionally efficient self-complementary (sc)AAVs, which do not require de novo second strand synthesis or intermolecular annealing for transgene expression. It has been demonstrated previously that the use of scAAV vectors rather than ssAAV greatly enhances transduction efficiency (McCarty, D. M. Self-complementary AAV vectors; advances and applications. Mol Ther 16, 1648-1656, doi: 10.1038/mt.2008.171 (2008)). Size restrictions also limited the choice of potential promoters for Cas9 expression. On the other hand, high levels of HSV gene editing were achieved in vitro using these same AAV/promoter/Cas9 constructs, so if true, these factors must be more important in vivo than in vitro. The low expression of sgRNA detected in SCG and TG (only 40% and 20%, respectively) may partially explained the poor gene editing in vivo. However, it is not the sole explanation, since m5 mRNA was detected in only 50% of the ganglia, yet the observed mutation reached up to 9.9% or 1.1% in SCG and TG, respectively. It cannot be ruled out that the disclosed in vitro model systems may not fully recapitulate the state of viral latency achieved in vivo, particularly in regard to viral chromatinization (reviewed in Knipe, D. M. Nuclear sensing of viral DNA, epigenetic regulation of herpes simplex virus infection, and innate immunity. Virology 479-480, 153-159, doi:10.1016/j.virol.2015.02.009 (2015)). However, the inventors have previously shown no detectable differences in the frequency of mutation induced by HSV-specific meganucleases in neuronal cultures established from TG of mice during acute vs. latent infection, which should differ in chromatinization. An intriguing possibility is that meganucleases might target highly compact and heterochromatinized viral genomes better than Cas9, which would be consistent with their evolution in eukaryotes, compared to the evolution of Cas9 in prokaryotes. Future studies should systematically evaluate expression of various classes of gene editing enzymes, and their efficacy against specific genomic and viral targets, to address this issue.

Taken together, these results provide strong support for the continued development of gene editing as a strategy against latent HSV infections, as well as other chronic infections such as HBV and HIV. A recent study using a similar approach for HIV shows eradication of detectable HIV in some (but not all) humanized mice (Dash, P. K. et al. Sequential LASER ART and CRISPR Treatments Eliminate HIV-1 in a Subset of Infected Humanized Mice. Nat Commun 10, 2753, doi:10.1038/s41467-019-10366-y (2019)). Similar studies have been performed in HBV-infected, liver-humanized mice, and achieved a near one-log reduction in intrahepatic HBV cccDNA levels, along with a remarkable improvement in human hepatocyte survival. (Stone, D. et al., CRISPR-Cas9 gene editing of hepatitis B virus in chronically infected humanized mice. Molecular Therapy Methods & Clinical Development. 2020 Nov. 26; 20:258-275. doi: 10.1016/j.omtm.2020.11.014. PMID: 33473359; PMCID: PMC7803634). Although the genetic diversity of these viruses remains a concern, careful informatics analysis can effectively address this issue (Roychoudhury, P. et al. Viral diversity is an obligate consideration in CRISPR/Cas9 designs for targeting the HIV reservoir. BMC Biol 16, 75, doi:10.1186/s12915-018-0544-1 (2018)). Moving this approach toward clinical application will require careful examination of its safety, including confirming the absence of off-target cleavage within the genome. The results also suggest important potential advantages for meganucleases, an underappreciated class of gene editing enzymes, in particular their compact nature that allows utilization of highly-optimized scAAV vectors and a wider selection of promoters. Although sequence-specific meganucleases are more difficult to develop compared with CRISPR/Cas9, this represents only a minor issue for targets such as HSV, which have limited genetic diversity and slow rates of genomic evolution. In principle, a handful of optimized meganucleases should be sufficient to cover the full diversity of HSV-1 and HSV-2 observed in human infection, in contrast to other less conserved viruses such as HBV and HIV (Roychoudhury, P. et al. Viral diversity is an obligate consideration in CRISPR/Cas9 designs for targeting the HIV reservoir. BMC Biol 16, 75, doi: 10.1186/s12915-018-0544-1 (2018); Schiffer, J. T. et al. Targeted DNA mutagenesis for the cure of chronic viral infections. J Virol 86, 8920-8936, doi: 10.1128/JVI.00052-12 (2012)). The levels of efficacy observed to date (>90% HSV reduction in SCG, and >50% in TG), if translated to humans, would be likely to meaningfully reduce HSV reactivation, shedding, transmission, and lesions. Further optimization of enzyme delivery and the meganucleases themselves are likely possible, and thus a cure for HSV infection may ultimately be within reach.

### Methods

### Cells and Herpesviruses

HEK293 (Graham, F. L., Smiley, J., Russell, W. C. & Nairn, R. Characteristics of a human cell line transformed by DNA from human adenovirus type 5. J Gen Virol 36, 59-74, doi: 10.1099/0022-1317-36-1-59 (1977)) and Vero cell lines (ATCC #CCL-81) were propagated in Dubelcco's modified Eagle medium supplemented with 10% fetal bovine serum. HSV-1 strain F (kindly provided by Dr J. Blaho) or syn17⁺ (kindly provided by Dr N. Sawtell) were used for the experiments and were propagated and titered on Vero cells.

### AAV Production and Titering

The following AAV vector plasmids were used to generate the AAV stocks in this study: pscAAV-CBh-m5, pscAAV-CBh-m8, pscAAV-CBh-m4, pssAAV-smCBA-m5-T2A-Trex2-2A-mCherry, pssAAV-sCMV-SaCas9-U6-sgRNA, pssAAV-CMV-SaCas9-U6-sgRNA, pssAAV-nEF-SaCas9-U6-sgRNA, pscAAV-CBh-NLS-mScarlet, pscAAV-CBh-NLS-mEGFP, pscAAV-CBh-NLS-DsRed-Express2, and pscAAV-CBh-NLS-mTagBFP2. AAV stocks of all serotypes were generated by transiently transfecting 293 cells using PEI at a ratio of 4:1 (μl PEI:μg DNA) according to the method of Choi et al., (Choi, V. W., Asokan, A., Haberman, R. A. & Samulski, R. J. Production of recombinant adeno-associated viral vectors for in vitro and in vivo use. Curr Protoc Mol Biol Chapter 16, Unit 16 25, doi:10.1002/0471142727.mb1625s78 (2007)). Briefly, 1.6×10⁷ HEK293 cells were transfected with 28 μg DNA comprised of the DNA for a scAAV or ssAAV vector plasmid, a plasmid that expresses the AAV rep and capsid proteins, and a helper plasmid that expresses adenovirus helper proteins (pHelper) at the ratio of 5:1:3, respectively. At 24 hours post-transfection media was changed to serum-free DMEM and after 72 hours cells were collected and re-suspended in AAV lysis buffer (50 mM Tris, 150 mM NaCl, pH 8.5) before freeze-thawing 4 times. AAV stocks were purified by iodixanol gradient separation (Choi, V. W., Asokan, A., Haberman, R. A. & Samulski, R. J. Production of recombinant adeno-associated viral vectors for in vitro and in vivo use. Curr Protoc Mol Biol Chapter 16, Unit 16 25, doi:10.1002/0471142727.mb1625s78 (2007); Zolotukhin, S. et al. Recombinant adeno-associated virus purification using novel methods improves infectious titer and yield. Gene Ther 6, 973-985, doi: 10.1038/sj.gt.3300938 (1999)) followed by concentration into PBS using an Amicon Ultra-15 column (EMD Millipore) and stored at −80° C. All AAV vector stocks were quantified by qPCR using primers/probe against the AAV ITR, with linearized plasmid DNA as a standard, according to the method of Aurnhammer et al. (Aurnhammer, C. et al. Universal real-time PCR for the detection and quantification of adeno-associated virus serotype 2-derived inverted terminal repeat sequences. Hum Gene Ther Methods 23, 18-28, doi: 10.1089/hgtb.2011.034 (2012)). AAV stocks were treated with DNase I and Proteinase K prior to quantification.

### Establishment of Neuronal Cultures

Neuronal cultures were established from TG harvested from mice at day 7 post-infection with 2×10⁵ PFU HSV-1(F) onto scarified cornea (Aubert, M. et al. In vivo disruption of latent HSV by designer endonuclease therapy. JCI Insight 1, doi:10.1172/jci.insight.88468 (2016)). Briefly, neuronal cultures were established after enzymatic digest with collagenase and dispase (Invitrogen, Carlsbad, CA) (Bertke, A. S. et al. A5-positive primary sensory neurons are nonpermissive for productive infection with herpes simplex virus 1 in vitro. J Virol 85, 6669-6677, doi:10.1128/JVI.00204-11 (2011)) and purification of the resulting cell homogenates using a percoll gradient (12.5% and 28%) (Malin, S. A., Davis, B. M. & Molliver, D. C. Production of dissociated sensory neuron cultures and considerations for their use in studying neuronal function and plasticity. Nature protocols 2, 152-160, doi:10.1038/nprot.2006.461 (2007)). Neurons were counted and plated on poly-D-lysine- and laminin-coated 12 mm round slides (BD Biosciences, San Jose CA) at a density of 4,000 neurons per well. Neurons were cultured without removing the non-neuronal cells that provide important growth support, and therefore these cultures contained a mixed population of neurons, satellite glial cells and other cell types. Cultures were maintained with complete neuronal medium, consisting of Neurobasal A medium supplemented with 2% B27 supplement, 1% PenStrep, L-glutamine (500 μM), and nerve growth factor (NGF; 50 ng/ml). Medium was replaced every 2-3 days with fresh medium. Acyclovir (100 nM, Sigma) was added to the culture medium for the first 5 days.

### HSV Infection and AAV Inoculation of Mice

Mice were housed in accordance with the institutional and NIH guidelines on the care and use of animals in research. 6-8 week old female Swiss Webster mice (Charles River) were used for all studies. For ocular HSV infection mice anesthetized by intraperitoneal injection of ketamine (100 mg per kg) and xylazine (12 mg per kg) were infected with 2×10⁵ PFU of HSV1(F) or syn17+ following corneal scarification of the right eye using a 28-gauge needle. For AAV inoculation, mice anesthetized with ketamine/xylazine were unilaterally administered the indicated AAV vector dose by either intradermal whisker pad (WP) injection, retro-orbital (RO) or tail-vein (TV) injection. The right (ipsilateral) TG and both SCGs were collected at the indicated time. Presence of AAV in ganglia of treated mice was confirmed by ddPCR (FIG. 12).

### Tissue Explant Reactivation

HSV was reactivated by incubating collected TG and SCG in 10% FBS-DMEM culture medium for 24 h, followed by total genomic DNA extraction as described below. After tissue reactivation, a statistically significant increase of 2 to 3-fold in HSV genomes is detected in reactivated compare to unreactivated (latent) tissues in untreated (control mice) (FIGS. 2G-2H and 10F).

### HSV Target Site PCR Amplification

Total genomic DNA (gDNA) was extracted using either the DNeasy Tissue & Blood micro kit (Qiagen, Valencia, CA) for neuronal cultures or the DNeasy Tissue & Blood mini kit (Qiagen, Valencia, CA) for whole TGs. Platinum Pfx DNA polymerase (Invitrogen, Carlsbad, CA) and 5 μl of gDNA were used to PCR amplify the region containing the target site for either HSV1m5 with U_{L}19 primers: forward 5′-CTGGCCGTGGTCGTACATGA (SEQ ID NO: 37) and reverse 5′-TCACCGACATGGGCAACCTT (SEQ ID NO: 38), HSV1m8 with UL30 primers: forward 5′-GAGAACGTGGAGCACGCGTACGGC (SEQ ID NO: 39) and reverse 5′-GGCCCGGTTTGAGACGGTACCAGC (SEQ ID NO: 40), HSV1m4 with ICP0 primers: forward 5′-GACAGCACGGACACGGAACT (SEQ ID NO: 41) and reverse 5′-TCGTCCAGGTCGTCGTCATC (SEQ ID NO: 42), SaCas9/sgRNA_{UL54} (sgRNA13, sgRNA17 and sgRNA26) with U_{L}54 primers: forward 5′-GACCGCATCAGCGAGAGCTT (SEQ ID NO: 43) and reverse 5′-CTCGCAGACACGACTCGAAC (SEQ ID NO: 44), or SaCas9/sgRNA_{UL30} (sgRNA1, and sgRNA10) with U_{L}30-primers: forward 5′-CGGCCATCAAGAAGTACGAG (SEQ ID NO: 45) and reverse 5′-AAGTGGCTCTGGCCTATGTC (SEQ ID NO: 46), with thermocycler conditions of: 94° C. 5 minutes, 40-45 cycles (94° C. 30 seconds, 60° C. 30 seconds, 70° C. 30 seconds), and then 70° C. 5 minutes.

### T7 Endonuclease 1 (T7E1) Assay

The T7 endonuclease assay and quantification to determine the levels of gene disruption were performed as follows. After PCR amplification of target site from HSV genomes, followed by purification using Zymo Research clean and concentrator-5 kit (Zymo Research, Irvine CA), 300 ng of DNA amplicon was denatured for 10 min at 95° C. and slowly reannealed by cooling down to room temperature. DNA was then digested with 5-10 units of T7 endonuclease (New England Biolabs,) for 30-60 min at 37° C. and resolved in an agarose gel. Quantification of gene disruption was performed using ImageJ software (NIH; Schneider, C. A., Rasband, W. S. & Eliceiri, K. W. NIH Image to ImageJ: 25 years of image analysis. Nat Methods 9, 671-675, doi: 10.1038/nmeth.2089 (2012)) and calculated using the formula: 100×(1−[1−fraction cleaved]½) where fraction cleaved=density of cleaved product/(density of cleaved product+density of uncleaved product).

ddPCR Quantification

Viral genome quantification by ddPCR was performed using an AAV ITR primer/probe set, and a gB primer/probe set for HSV as described previously (Aubert, M. et al. In vivo disruption of latent HSV by designer endonuclease therapy. JCI Insight 1, doi:10.1172/jci.insight.88468 (2016)). Cell numbers in tissues were quantified by ddPCR using mouse-specific RPP30 primer/probe set: For 5′-GGCGTTCGCAGATTTGGA (SEQ ID NO: 47), Rev 5′-TCCCAGGTGAGCAGCAGTCT (SEQ ID NO: 48), probe 5′-ACCTGAAGGCTCTGCGCGGACTC (SEQ ID NO: 49). In some control ganglia, sporadic samples showed positivity for AAV genomes, although at levels typically >2-3 logs lower than ganglia from treated mice that had received AAV. This can be attributed to low-level contamination of occasional tissue samples.

### Illumina Next Generation Sequencing (NGS)

Next generation sequencing of meganuclease target sites was performed using PCR products generated with the target site-specific primers described above and a MiSeq sequencer (Illumina) (Aubert, M. et al. In vivo disruption of latent HSV by designer endonuclease therapy. JCI Insight 1, doi:10.1172/jci.insight.88468 (2016)).

### Single Cell RNA Analysis

Swiss-Webster mice were latently infected with 10⁵ PFU HSV-1 syn 17+ via the ocular route, and after 60 days injected with one of 4 different AAV serotypes: 1, 8, PHP.S, and Rh10, each carrying a unique fluorescent protein transgene: mScarlet, mEGFP, DsRed.Express2, and TagBFP2 respectively under the CBh promoter. For each serotype three mice were independently injected with 10¹² AAV genomes subcutaneously in the whisker pad (AAV1) or intravenously in the retro-orbital vein (AAV8, PHP.S, and Rh10). Three weeks later, TG and SCG from animals were collected and each tissue (TG or SCG) was pooled from all animals for neuron isolation via enzymatic tissue digest (see above), followed by density gradient centrifugation and enrichment using the Neuron Isolation Kit (Miltenyi BioTech.), which allows untouched neurons to flow through the column while non-neuronal cells remain bound.

Only 2 AAV8-mEGFP animals were used for cell preparations or analyses. Tissue and isolated neurons were maintained in ice cold Neurobasal A medium supplemented with 2% B27 supplement, 1% PenStrep, L-glutamine (500 μM) or PBS throughout the procedure except during the enzymatic tissue digestion steps. Cells were encapsulated and scRNA-seq libraries were prepared in the Genomics Core Facility at the FHCRC using the Chromium Single Cell 3′ Library and Gel Bead Kit v2 from 10× Genomics according to manufacturer instructions. 10× Genomics Single Cell 3′ expression libraries were sequenced on an Illumina HiSeq 2500 running in High-Output mode with a paired end (26 bp×8 bp×98 bp) sequencing strategy. The SCG and TG libraries were pooled and distributed over 8 sequencing lanes. Image analysis and base calling were performed using RTA Version 1.18.66.3. Sequencing reads were processed with the 10× Genomics ‘Cell Ranger’ v2.1.0 and with Seurat v2.3.4 (Butler, A., Hoffman, P., Smibert, P., Papalexi, E. & Satija, R. Integrating single-cell transcriptomic data across different conditions, technologies, and species. Nat Biotechnol 36, 411-420, doi:10.1038/nbt.4096 (2018)). High quality sequence data was obtained from 2,372 purified TG neurons and 2, 172 SCG neurons.

### Statistics and Reproducibility

GraphPad Prism 7 software was used for statistical analyses. Comparison of HSV loads was performed with multiple t-test, with alpha-0.05%. Each tissue was analyzed individually, without assuming a consistent SD. HSV and AAV distributions in scRNA-seq experiments were analyzed using the χ² test with alpha=0.05. Because tissue from animals injected with the different AAV serotype/transgene were pooled, cell counts were normalized to input by multiplying cell count by number of animals receiving that serotype divided by total number of animals. The mean and error bars representing the standard deviation are shown on each graph. The findings of the studies were reproduced across the experiments using different experimental set-ups.

RT-ddPCR Quantification of SaCas9, sgRNA, HSV1m5 Expression

AllPrep DNA/RNA kit (Qiagen, Valencia, CA) was used to isolate DNA and RNA from ganglia collected in experiment presented in FIG. 4. SaCas9, sgRNA and HSV1m5 expression was quantified with One-step RT-ddPCR kit (BIO-RAD, Hercules, CA) using 2 μl of RNA and the following primers/probe set: SaCas9 specific primers: SaCas9 forward 5′-CCGCCCGGAAAGAGATTATT (SEQ ID NO: 5′-50), reverse CGGAGTTCAGATTGGTCAGTT (SEQ ID NO: 51), and probe [FAM]AGCTGCTGGATCAGATTGCCAAGA[MGB] (SEQ ID NO: 52); Tracr specific primers: TRACR LS forward 5′-TGCCGTGTTTATCTCGTCAACT (SEQ ID NO: 53), reverse 5′-CCCGCCATGCTACTTATCTACTTAA (SEQ ID NO: 54), and probe [FAM]TTGGCGAGATTTTT[MGB] (SEQ ID NO: 55); HSV1m5 specific primers: m5 mega forward 5′-TGGACAGCCTGAGCGAGAA (SEQ ID NO: 56), reverse 5′-GCAGAGACAGAGGAGCAATGTG (SEQ ID NO: 57), and probe

[FAM]CGGCCGGTGATTCCTCTGTTTCTAATTC[BHQ] (SEQ ID NO: 58). The cycling steps were as followed: reverse transcription 50° C. 60 minutes, enzyme activation 95° C. 10 minutes, 40 cycles (95° C. 30 seconds, 60° C. 1 minute, 70° C. 30 seconds), and then enzyme deactivation 98° C. 10 minutes.

### Example 2

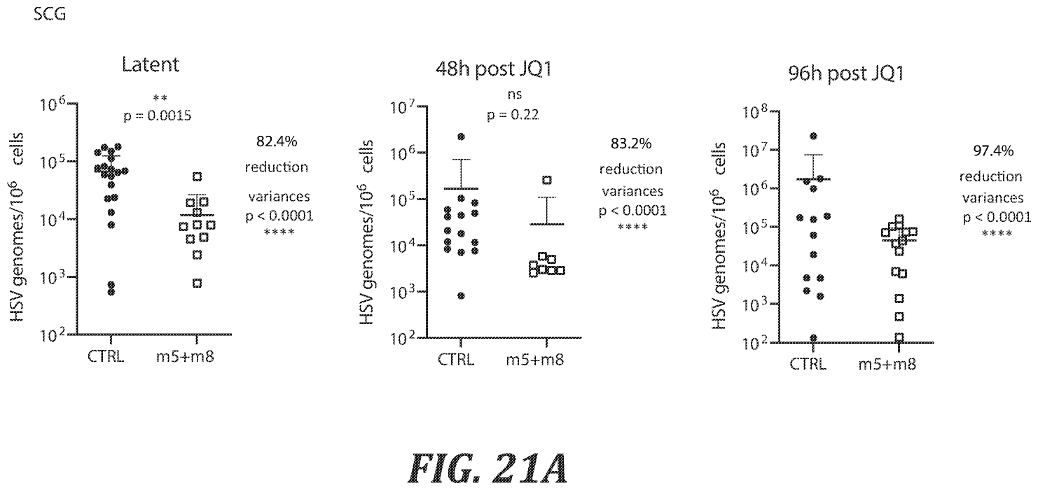

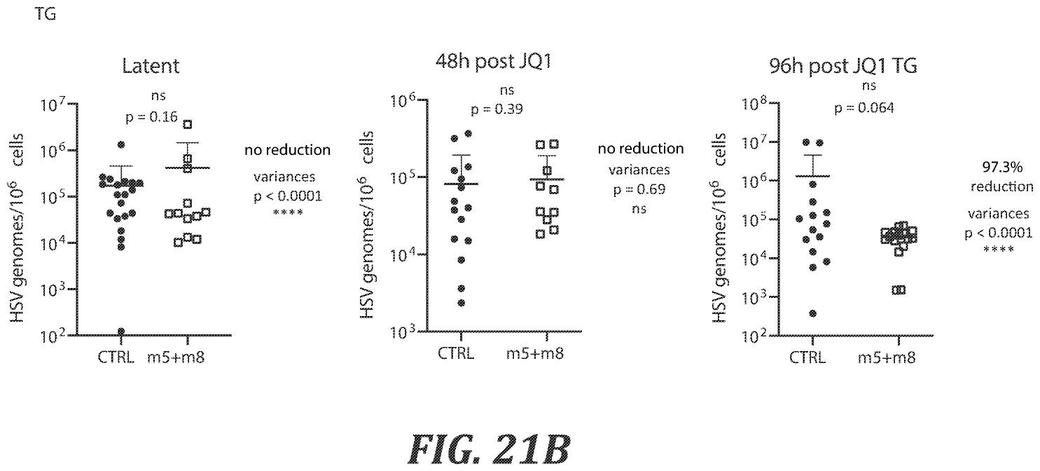

This Example describes that the use of meganucleases, as described in Example 1, combined with a bromodomain and extra-terminal motif (BET) protein inhibitor dramatically reduced viral load. FIG. 21.

It is known that BET inhibitors reactivate HSV-1 in vivo and in vitro. (Ren, K., et al., An Epigenetic Compound Library Screen Identifies BET Inhibitors that Promote HSV-1 and -2 Replication by Bridging P-TEFb to Viral Gene Promoters through BRD4, PLOS Pathogens, Oct. 20, 2016, https://doi.org/10.1371/journal.ppat. 1005950; Alfonso-Dunn, R., et al., Transcriptional Elongation of HSV Immediate Early Genes by the Super Elongation Complex Drives Lytic Infection and Reactivation from Latency, Cell Host & Microbe, vol. 21, issue 4, Apr. 12, 2017, 507-517.e). However, Ren et al., and Alfonso-Dunn et al., did not investigate whether the BET inhibitor, JQ1 caused mice to shed virus from their mucosal surfaces-mice do not shed spontaneously. The inventors determined that JQ1 caused mice to shed virus from their mucosal surfaces, which allowed for their mouse model to be used in shedding experiments to ask whether meganucleases would reduce BET mediated shedding and whether the combination of meganucleases and BET inhibitors would reduce HSV-1 load.

### JQ1 Treatment Leads to Shedding

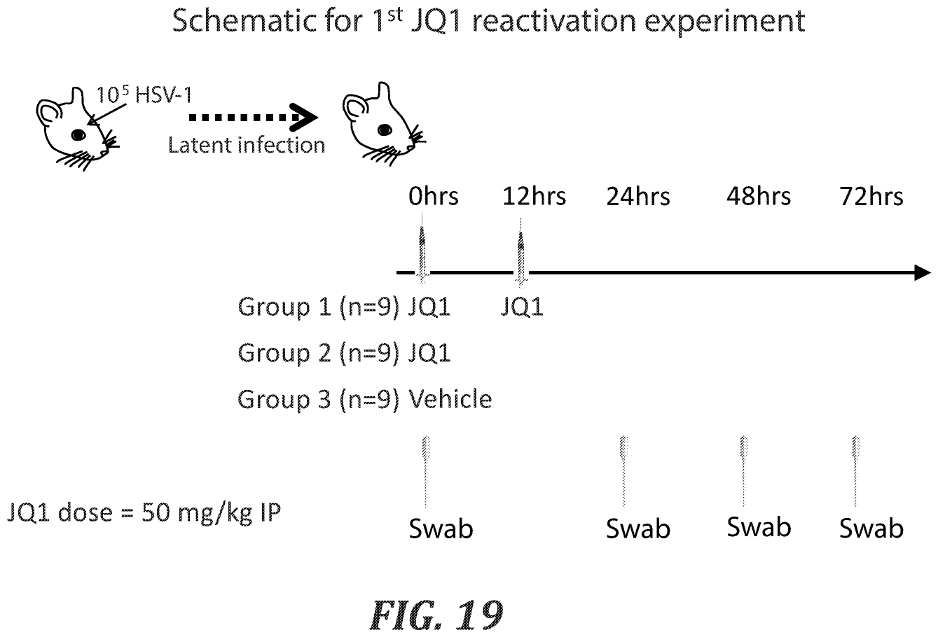

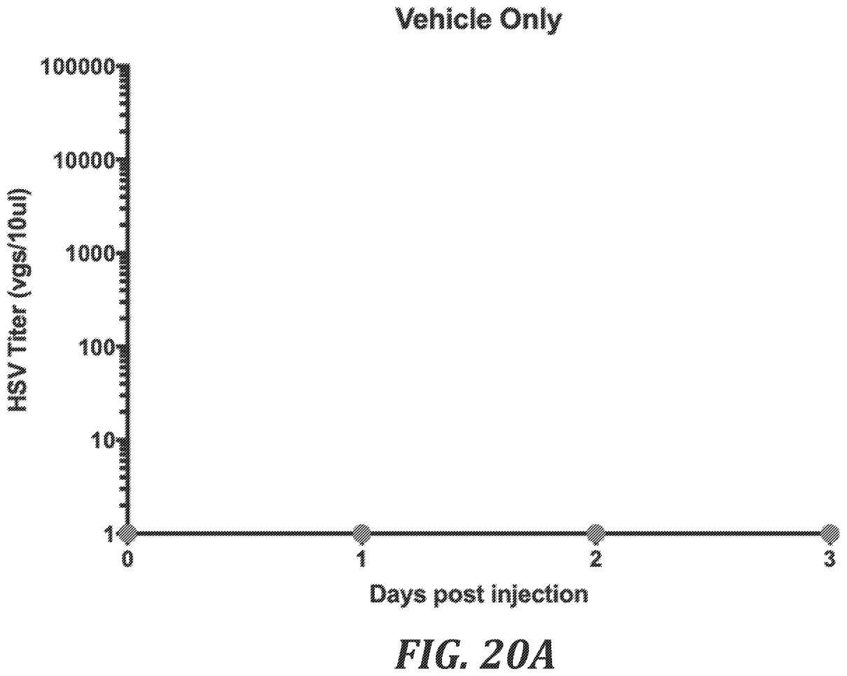

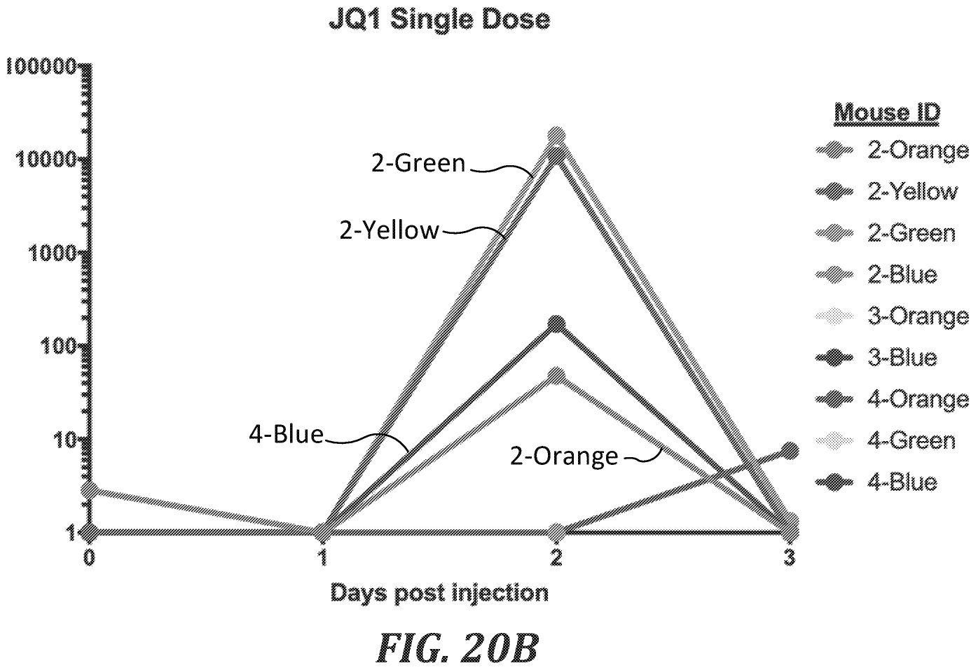

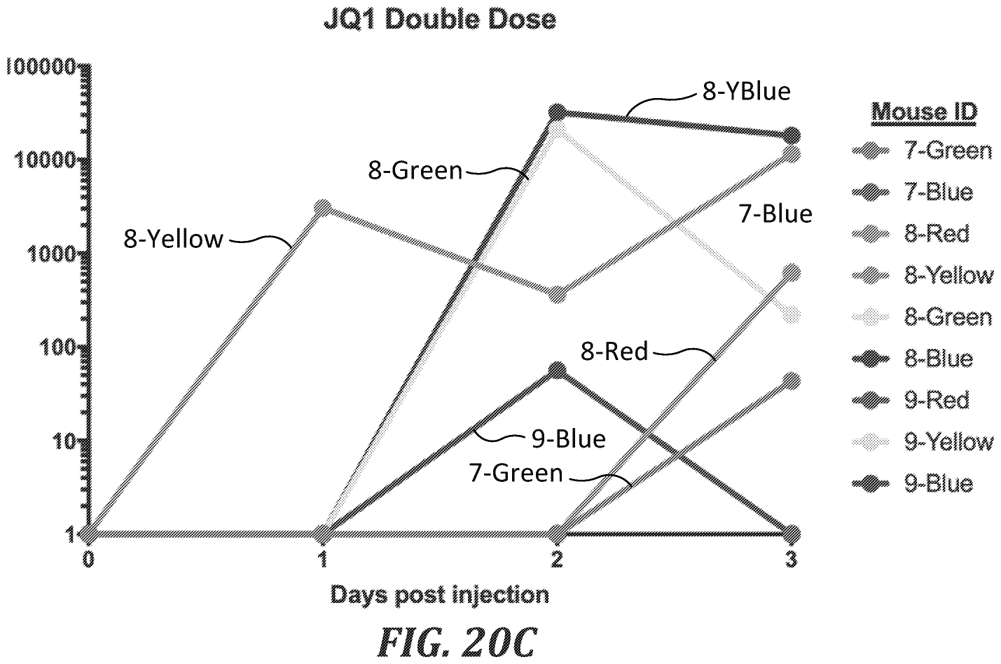

To begin, the mouse model for BET induced shedding was verified. In this model, latent infected mice were divided into three experimental groups to determine the effect of the BET inhibitor, JQ1, on HSV-1 reactivation. FIG. 19. As illustrated, Group 1 and Group 2 were JQ1 groups that received 50 mg/kg intraperitoneal injection of JQ1. Group 3 was the control group, i.e., no JQ1 injection. Group 1 mice received two injections, the first JQ1 injection at 0 hrs and the second JQ1 injection at 12 hrs. Group 2 mice received one injection at 0 hrs. Group 3 mice received one control injection at 0 hrs. To determine shedding from mucosal surfaces, as an indicator of HSV-1 reactivation, mice were swabbed at 0 hrs (control) and this swab was compared to swabs taken at 24 hours, 48 hours, and 72 hours. As illustrated in FIGS. 20A-20C, no shedding was detected in the control mice (FIG. 20A, Group 3 FIG. 19). However, shedding was detected in the JQ1 single dose mice (Group 2 FIG. 19), which peaked 2 days following the JQ1 injection. FIG. 20B. Further, in FIG. 20C, shedding was also detected in the mice that received two doses of JQ1 (Group 1 FIG. 19). These data indicate that JQ1 causes mice to shed virus from their mucosal surfaces and the onset and duration of shedding appear to be dependent on JQ1 in a dose dependent manner. Compare the onset and duration of viral shedding in JQ1 double dose to JQ1 single dose.

### Meganuclease and JQ1 Treatment Reduced Viral Load

The shedding mouse model (FIG. 19) was used to determine whether meganuclease treatment reduces shedding induced by JQ1 and whether treatment with meganucleases and JQ1 reduced HSV loads. Meganuclease therapy reduced JQ1 shedding by >95% (data not shown). To determine whether the combination of a meganuclease with JQ1 reduced HSV-1 load, latent infected mice were separated into a SCG group and a TG group. FIG. 21. In each group mice were further divided into three treatment groups: (1) latent; (2) 48 hours post JQ1 treatment; and (3) 96 hours post JQ1 treatment. Each treatment group had a control and treatment with the dual-meganuclease (m5+m8) as described in Example 1. As illustrated in the latent treatment group, i.e., no JQ1 treatment, meganuclease treatment did not reduce viral load in TG cells as compared to control. FIG. 21B. In SCG cells, meganuclease treatment did reduce viral load 82.4% compared to control. FIG. 21A. In the 48 hours post JQ1 treatment groups, meganuclease+JQ1 treatment reduced viral load 83.2% compared to control in SCG cells (FIG. 21A) but had no effect in TG cells (FIG. 21B). However, in the 48 hours post JQ1 treatment groups, meganuclease+JQ1 reduced viral load 97.4% in SCG cells (FIG. 21A) and surprisingly reduced viral load 97.3% in TG cells (FIG. 21B). Therefore, these data indicate that the BET inhibitor, JQ1, can be used to increase meganuclease editing, especially in TG cells where editing has been observed to be weaker.

The embodiments of the invention in which an exclusive property or privilege is claimed are defined as follows:

1. A method for reducing or eliminating latent Herpes simplex virus type 1 (HSV-1) reactivation in a cell, the method comprising delivering to an HSV-1-infected cell one or more viral vectors comprising one or more sequences encoding one or more HSV-1-specific meganucleases.

2. The method of claim 1, wherein the one or more viral vectors is a self-complementary adeno-associated virus (scAAV) and/or a single-stranded adeno-associated virus (ssAAV).

3. The method of claim 2, wherein the one or more viral vectors is scAAV.

4. The method of claim 3, wherein the one or more scAAVs comprises AAV-Rh10, AAV8, AAV1 serotype adeno-associated virus, or a combination thereof.

5. The method of claim 3 or claim 4, wherein the one or more scAAVs comprises AAV-Rh10 or AAV8 serotype adeno-associated virus.

6. The method of any one of claims 1-5, wherein the one or more HSV-1-specific meganucleases are configured to induce one or more DNA double strand breaks (DSB).

7. The method of any one of claims 1-6, wherein the one or more HSV-1-specific meganucleases is a meganuclease configured to target one or more HSV-1 genes essential for replication.

8. The method of any one of claims 1-7, wherein the one or more meganucleases comprise a sequence as set forth in SEQ ID NOs: 1-3.

9. The method of any one of claims 1-8, wherein the one or more meganucleases is a meganuclease configured to target one or more sequences as set forth in SEQ ID NOs: 4-6.

10. The method of any one of claims 3-9, wherein the method comprises delivering one scAAV comprising two or more sequences encoding one or more HSV-1-specific meganucleases.

11. The method of any one of claims 3-9, wherein the method comprises delivering two scAAVs each comprising one or more sequences encoding one or more HSV-1-specific meganucleases.

12. The method of any one of claims 3-9, wherein the method comprises delivering two different scAAVs each comprising a sequence encoding an HSV-1-specific meganuclease, wherein the sequences are the same or different.

13. The method of any one of claims 1-12, wherein the cell is in a mammalian subject.

14. The method of claim 13, wherein the mammalian subject is human.

15. The method of any one of claims 1-14, wherein the cell is a superior cervical ganglia (SCG) cell, a trigeminal ganglia (TG) cell, or a combination thereof.

16. The method of any one of claims 13-15, wherein the one or more scAAVs is delivered with one or more pharmaceutically acceptable carriers configured for injection.

17. The method of claim 16, wherein the one or more scAAVs is delivered to the subject by a subcutaneous injection.

18. The method of claim 16, wherein the one or more scAAVs is delivered to the subject by an intramuscular injection.

19. The method of any one of claims 1-18, wherein the method further comprises administering to the HSV-1-infected cell a bromodomain and extra-terminal (BET) protein inhibitor.

20. The method of claim 19, wherein the method comprises administering the BET protein inhibitor to the HSV-1-infected cell before delivering to the HSV-1-infected cell one or more scAAVs comprising one or more sequences encoding one or more HSV-1-specific meganucleases.

21. The method of claim 19, wherein the method comprises administering the BET protein inhibitor to the HSV-1-infected cell after delivering to the HSV-1-infected cell one or more scAAVs comprising one or more sequences encoding one or more HSV-1-specific meganucleases.

22. The method of claim 19, wherein the method comprises administering the BET protein inhibitor to the HSV-1-infected cell concomitant with delivering to the HSV-1-infected cell one or more scAAVs comprising one or more sequences encoding one or more HSV-1-specific meganucleases.

23. The method of any one of claims 19-22, wherein the BET protein inhibitor is administered at a dose sufficient to provide a concentration of 3 μM or less in the HSV-1-infected cell.

24. The method of any one of claims 19-23, wherein the BET protein inhibitor is selected from the group of JQ1, birabresib, molibresib, apabetalone, ZEN-3694, BMS-986158, and INC-B057643.

25. The method of any one of claims 19-24, wherein the method reduces HSV-1 load at least 97% in the SCG cell at least 96 hours following administration of the BET protein inhibitor.

26. The method of any one of claims 19-24, wherein the method reduces HSV-1 load at least 83% in the SCG cell at least 48 hours following administration of the BET protein inhibitor.

27. The method of any one of claims 19-24, wherein the method reduces HSV-1 load at least 97% in the TG cell 96 hours following administration of the BET protein inhibitor.

28. A composition for reducing or eliminating latent HSV-1 reactivation in a cell, the composition comprising one or more viral vectors comprising one or more sequences encoding one or more HSV-1-specific meganucleases.

29. The composition of claim 28, wherein the one or more viral vectors is a self-complementary adeno-associated virus (scAAV) and/or a single-stranded adeno-associated virus (ssAAV).

30. The composition of claim 29, wherein the one or more viral vectors is scAAV.

31. The composition of claim 30, wherein the one or more scAAVs is AAV-Rh10, AAV8, AAV1 serotype adeno-associated virus, or a combination thereof.

32. The composition of claim 30 or claim 31, wherein the one or more scAAVs is AAV-Rh10 or AAV8 serotype adeno-associated virus.

33. The composition of any one of claims 28-32, wherein the one or more HSV-1-specific meganucleases are configured to induce one or more DNA double strand breaks (DSB).

34. The composition of any one of claims 28-33, wherein the one or more HSV-1-specific meganucleases is a meganuclease configured to target one or more HSV-1 genes essential for replication.

35. The composition of any one of claims 28-34, wherein the one or more meganucleases is a meganuclease comprises a sequence as set forth in SEQ ID NOs: 1-3.

36. The composition of any one of claims 28-35, wherein the one or more meganucleases is a meganuclease configured to target one or more sequences as set forth in SEQ ID NOs: 4-6.

37. The composition of any one of claims 28-36, wherein the composition comprises one or more pharmaceutically acceptable carriers configured for subcutaneous injection.

38. The composition of any one of claims 28-37, wherein the composition further comprises a bromodomain and extra-terminal (BET) protein inhibitor.

39. The composition of claim 38, wherein the BET protein inhibitor is administered at a dose sufficient to provide a concentration of at least 3 μM or less in an HSV-1-infected cell.

40. The composition of claim 38 or claim 39, wherein the BET protein inhibitor is selected from the group of JQ1, birabresib, molibresib, apabetalone, ZEN-3694, BMS-986158, and INC-B057643.

41. The composition of any one of claims 38-40, wherein the composition reduces HSV-1 load at least 97% in a superior cervical ganglia cell (SCG) 96 hours following administration of the BET protein inhibitor.

42. The composition of any one of claims 38-40, wherein the composition reduces the HSV-1 load at least 83% in the SCG cell 48 hours following administration of the BET protein inhibitor.

43. The composition of any one of claims 38-40, wherein the composition reduces HSV-1 load at least 97% in a trigeminal ganglia cell (TG) 96 hours following administration of the BET protein inhibitor.
